SEQUENCE LISTING (5 sequences)

SEQ ID NO: 1 - protein, 13 aa, synthetic construct
  REGION 1..13: note = Synthetic Polypeptide
GRKKRRQRRRPPQ

SEQ ID NO: 2 - protein, 11 aa, synthetic construct
  REGION 1..11: note = Synthetic Polypeptide
YGRKKRRQRRR

SEQ ID NO: 3 - protein, 5 aa, synthetic construct
  REGION 1..5: note = Synthetic Polypeptide
GWWWG

SEQ ID NO: 4 - protein, 5 aa, synthetic construct
  REGION 1..5: note = Synthetic Polypeptide
GFWFG

SEQ ID NO: 5 - protein, 4 aa, synthetic construct
  REGION 1..4: note = Synthetic Polypeptide
GWWG

(19) United States
(12) Patent Application Publication
(10) Pub. No.: US 2025/0387471 A1
(43) Pub. Date: Dec. 25, 2025

(54) METHODS AND COMPOSITIONS FOR MODULATION OF IMMUNE RESPONSES

(71) Applicant: THE UNIVERSITY OF CHICAGO, Chicago, IL (US)

(72) Inventors: Aaron ESSER-KAHN, Chicago, IL (US); Saikat MANNA, Chicago, IL (US); Sampa MAITI, Chicago, IL (US); Adam WEISS, Chicago, IL (US)

(21) Appl. No.: 18/879,670

(22) PCT Filed: Jun. 29, 2023
(86) PCT No.: PCT/US2023/069385

§ 371 Date: Dec. 27, 2024

Related U.S. Application Data
(60) Provisional application No. 63/356,711, filed on Jun. 29, 2022.

(51) Int. Cl. A61K 39/385; A61K 39/00; A61K 39/39; A61P 37/04

(52) U.S. Cl. CPC A61K 39/385; A61K 39/0011; A61K 39/39; A61P 37/04; A61K 2039/55555; A61K 2039/55572; A61K 2039/876

(57) ABSTRACT

Aspects of the present disclosure relate to functionalized polymers and methods of use thereof. Certain aspects are directed to polymers comprising adjuvants for use in stimulating an immune response. In some cases, provided are polymers comprising inflammasome activators, in some cases also comprising a TLR agonist, which may be formulated in a pharmaceutical composition. Also disclosed are methods for improving vaccine efficacy and immunotherapy efficacy. Certain aspects relate to compositions and methods for stimulation of CD4+ and/or CD8+ T cell responses in a subject.

### CROSS-REFERENCE TO RELATED APPLICATIONS

This application claims the benefit of priority to U.S. Provisional Patent Application Ser. No. 63/356,711, filed Jun. 29, 2022, which is hereby incorporated by reference in its entirety.

### STATEMENT REGARDING FEDERALLY SPONSORED RESEARCH OR DEVELOPMENT

This invention was made with government support under AI124286 awarded by the National Institutes of Health. The government has certain rights in the invention.

### SEQUENCE LISTING

The instant application contains a Sequence Listing which has been submitted in XML format and is hereby incorporated by reference in its entirety. Said XML copy, created on Jun. 28, 2023, is named ARCDP0692WO Sequence Listing and is 7 kilobytes in size.

### BACKGROUND

### I. Field of the Invention

This invention relates generally to the fields of biochemistry, immunology, and medicine.

### II. Background

A major focus of vaccine adjuvant development has been on the development of robust antibody-mediated protection against pathogens. However, such adjuvants have failed to elicit a robust T-cell mediated protection. As such, various pathogen associated molecular patterns (PAMPs) are being investigated to target antigen presenting cells (APCs) to initiate a robust downstream adaptive immune response. Research in this area has focused on targeting Toll-Like Receptor agonists (TLRs). Presentation of these agonists with tailored physicochemical properties and altered bioavailability has been shown to generate robust antigen-specific T-cell response.

Personalized cancer vaccines that target tumor specific mutations (‘neo-antigens’) hold enormous promise for tumor treatment. However, despite numerous promising results, the low immunogenicity of neoantigens peptides remains a major roadblock that deters further clinical adoption. To address the challenge, various strategies have been investigated for the development of immunostimulant or adjuvant platforms that enhance immune recognition by antigen presenting cells (APCs). In this context, adjuvants such as CpG, 2BXy, poly ICLC and STING have been employed in design of various adjuvant platforms to enhance immunogenicity. Unfortunately, such a strategy can target only one single pathogen sensing pathway. In contrast, natural pathogens activate more than one sensing pathways-often in distinct combinations to elicit highly amplified immune stimulation in APCs.

The NLRP3 inflammasome is a multiple subunit protein complex that, upon activation, leads to the generation of active caspase-1 resulting in the release of active interleukin-1β (IL-1β) and interleukin-18 (IL-18), causing a voluntary form of cell death known as pyroptosis. The role of the NLRP3 inflammasome is known in innate immunity for pathogen recognition and clearance. Recent reports have shed light into the mechanism and sub-temporal orchestration of inflammasome activation in vivo. In addition, there is compelling evidence that inflammasome activation in APCs contributes to anti-tumor responses via secretion of key cytokines IL-1β and IL-18.

Recognized herein is a need for adjuvant compositions having robust, immune activating activity and limited toxicity. Also recognized are methods for use of such compositions in enhancing an immune response, including a vaccine-based immune response and an anti-cancer immune response.

### SUMMARY

Disclosed herein are methods and compositions related to formulation and delivery of various agents, including polypeptides. Certain embodiments relate to functionalized polymers for delivery of therapeutic polypeptides, including adjuvants. In some embodiments, inflammasome activators are provided conjugated to one or more polymers for use as an immune activator and/or vaccine adjuvant. A polymer may serve as a non-immunogenic scaffold for an inflammasome activator. Inflammasome activators include NLRP3 inflammasome-activating polypeptides. An inflammasome activator may comprise an NLRP3 inflammasome-activating polypeptide and a TLR agonist. In some aspects, a NLRP3 inflammasome-activating polypeptide comprises a cell penetrating peptide (e.g., HIV TAT) and an endosomal escape polypeptide (e.g., GWWWG). In some aspects, the disclosed methods and compositions are useful in stimulating an immune response for improving efficacy of a vaccine such as a cancer vaccine.

Aspects of the present disclosure include compounds, molecules, monomers, polymers, polypeptides, PRR agonists, TLR agonists, TLR7/8 agonists, NF-κB inhibitors, immune modulators, immunotherapeutics, nanoparticles, polymer synthesis methods, methods for nanoparticle generation, immune activation methods, vaccination methods, cancer treatment methods, and cancer prevention methods. Certain aspects are directed to polymers comprising an inflammasome activator and/or a TLR agonist. Additional aspects are directed to methods for use of such polymers, including methods for enhancing an immune response to an antigen (e.g., in a vaccine), an immunotherapy (e.g., a cancer immunotherapy), or other immune stimulation.

Compounds of the present disclosure include, for example, polymers, polypeptides, TLR agonists (including TLR7/8 agonists), immune modulator agents, and inflammasome activators. A compound (e.g., polymer) of the disclosure can comprise at least 1, 2, 3, or more of: a TLR agonist, a linker, a polypeptide, an adjuvant, a cell penetrating peptide, and an inflammasome activator. Any one or more of these components may be excluded from a compound of the disclosure in certain aspects.

Methods of the present disclosure include, for example, treatment methods, disease prevention methods, vaccination methods, synthesis methods, immune activation methods, cellular activation methods, and CD4+ T cell activation methods. A method of the present disclosure can include at least 1, 2, 3, or more of the following steps: synthesizing a polymer, generating a nanoparticle, administering a polymer, administering an immune modulator, administering an adjuvant, administering an antigen, generating a pharmaceutical composition comprising an antigen and an immune modulator, diagnosing a subject as having cancer, diagnosing a subject as having a viral infection, diagnosing a subject as having a bacterial infection, diagnosing a subject as having a parasitic infection, diagnosing a subject as having an autoimmune condition, and administering a cancer therapy, an anti-viral therapy, an anti-bacterial therapy, and/or an anti-parasitic therapy. Any one or more of these steps may be excluded from a method of the disclosure in certain aspects. In some aspects, the subject or cell is human.

Disclosed herein, in some aspects, is a polymer of formula (I)

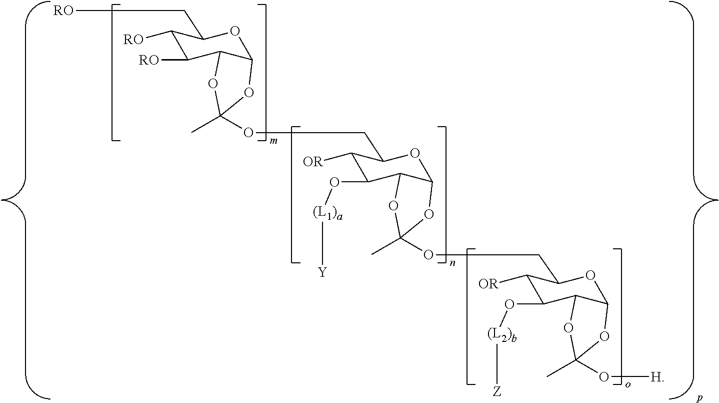

wherein
each R is independently H, alkyl, or acyl;
m is an integer ranging from 0 to 10;
n is an integer ranging from 0 to 10;
is an integer ranging from 0 to 10;
p is an integer ranging from 1 to 500;
L₁ is a first linker;
Z comprises a first polypeptide;
L₂ is a second linker;
Y comprises an adjuvant;
a is 0 or 1; and
b is 0 or 1;
wherein n+o≥1.

In some aspects, L₁ and L₂ each independently comprise at least one of a maleimide moiety, a polyethylene glycol (PEG) moiety, and a triazole moiety. In some aspects, n is 0, 1, 2, 3, 4, 5, 6, 7, 8, 9, or 10. In some aspects, n is 0. In some aspects, o is 0, 1, 2, 3, 4, 5, 6, 7, 8, 9, or 10. In some aspects, o is 0. In some aspects, n is equal to o. In other aspects, n is not equal to o.

In some aspects, Z is of formula (II):

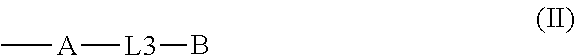

wherein A is the first polypeptide, L₃ is a third linker, and B is a second polypeptide.

In some aspects, A is an agent capable of rupturing an endosome or a lysosome. In some aspects, A is a cell penetrating peptide. In some aspects, the cell penetrating peptide is a TAT peptide, penetratin, transportan, MAP, Pep-1, Pept 1, Pept 2, IVV-14, pVEC, HRSV, or polyarginine. In some aspects, the cell penetrating peptide comprises a sequence from an HIV TAT protein. In some aspects, the sequence is a sequence from amino acids 45-65 of HIV-1 TAT protein. In some aspects, the sequence is amino acids 48-60 of HIV-1 TAT protein (SEQ ID NO: 1). In some aspects, the sequence is amino acids 47-57 of HIV-1 TAT protein (SEQ ID NO: 2). In some aspects, B is a hydrophobic endosomal escape peptide. In some aspects, the hydrophobic endosomal escape peptide has the sequence GWWWG (SEQ ID NO: 3), GFWFG (SEQ ID NO: 4), or GWWG (SEQ ID NO: 5). In some aspects, L₃ is a polyethylene glycol (PEG) linker comprising c ethyleneoxy units. In some aspects, L₃ is a N-(2-hydroxypropyl)-methacrylamide (HPMA) linker, a PEG-methylacrylamide (PEGMA) linker, a succinimide linker, a maleimide linker, a polyamide linker, a polyester linker, or a bifunctional or trifunctional linker comprising a combination of the aforementioned linkers. In some aspects, the linker comprises c monomeric units. In some aspects, c is between 2 and 20. In some aspects, c is 6. In some aspects, c is 2, 3, 4, 5, 6, 7, 8, 9, 10, 11, 12, 13, 14, 15, 16, 17, 18, 19, or 20.

In some aspects, Z comprises an inflammasome activator. In some aspects, the inflammasome activator is capable of activating an NLRP3 inflammasome. In some aspects, the polymer is capable of stimulating IL-1β production in a subject. In some aspects, the polymer is capable of stimulating activation and/or proliferation of CD4+ T cells in a subject. In some aspects, the adjuvant is a TLR agonist. In some aspects, the TLR agonist is a TLR7 agonist. In some aspects, the TLR agonist is a TLR4 agonist. In some aspects, the TLR agonist is 2Bxy. In some aspects, the TLR agonist is a functionalized 2Bxy. In certain aspects, the polymer further comprises an additional adjuvant. In some aspects, the additional adjuvant is attached to the same polymer residue as the first polypeptide. In some asects, the subject or cell is human.

In some aspects, the polymer is of formula (III):

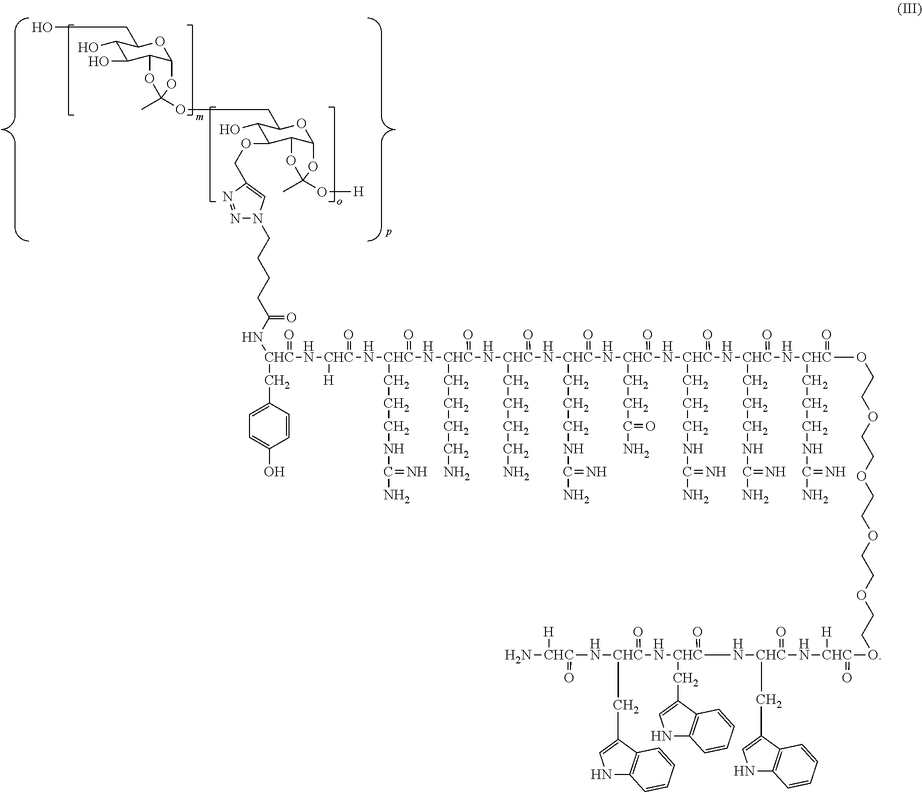

In some aspects, the polymer is capable of stimulating activation and/or proliferation of CD8+ T cells in a subject. In some aspects, at least one R group is an acetyl group.

In some aspects, the polymer is of formula (IV):

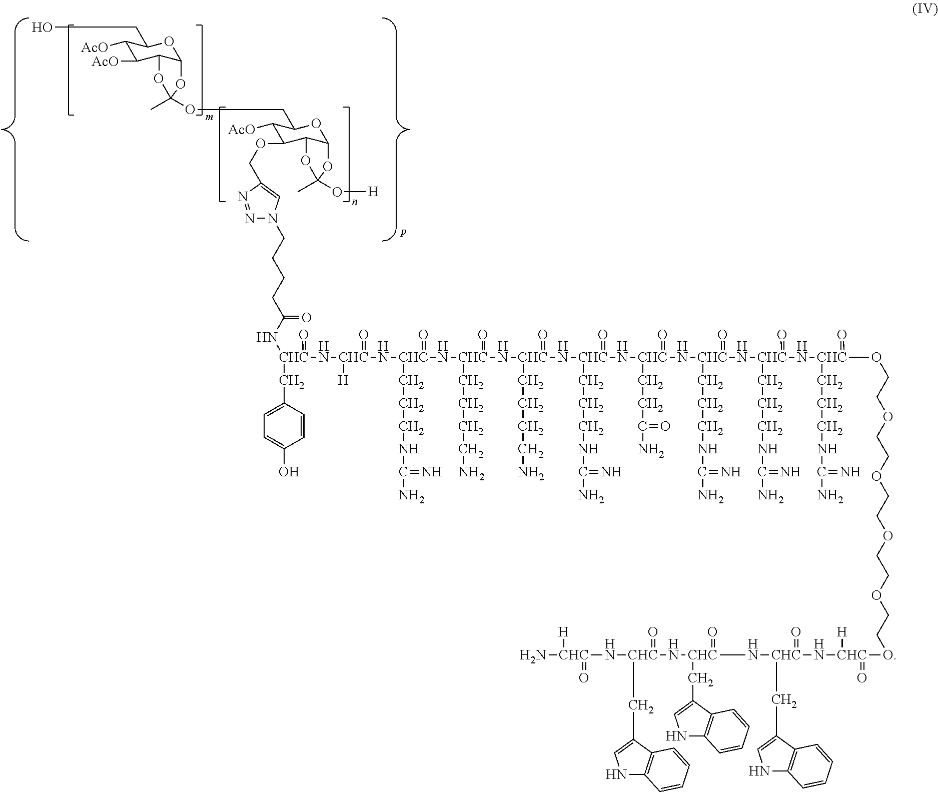

In some aspects, the polymer is of formula (V):

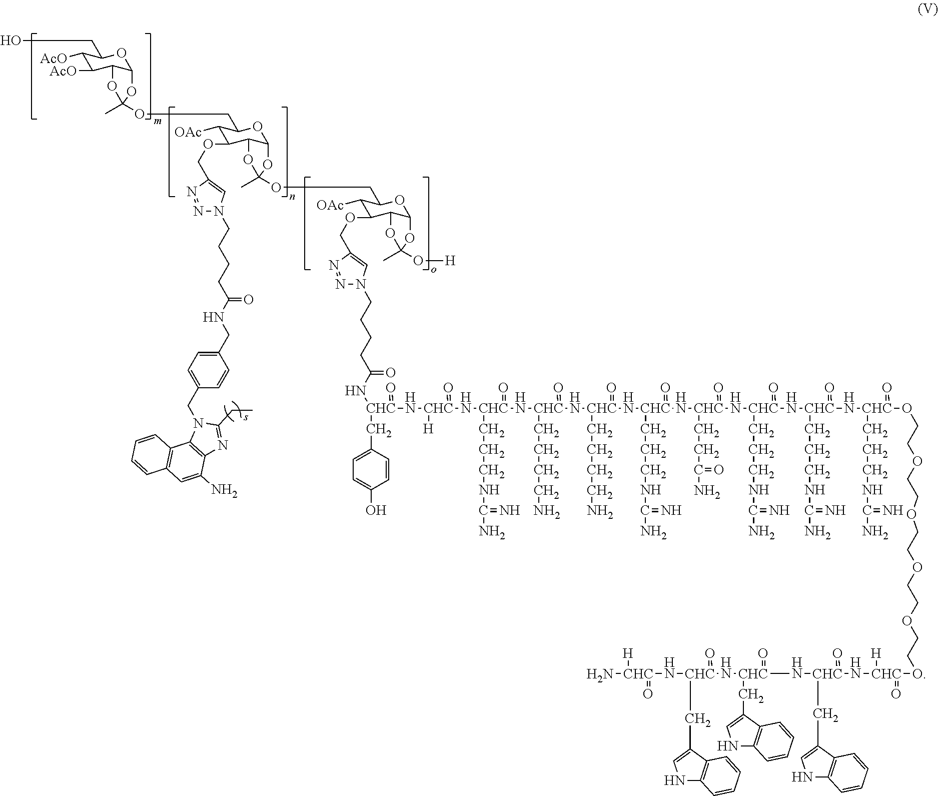

In some aspects, the polymer is of formula (VI):

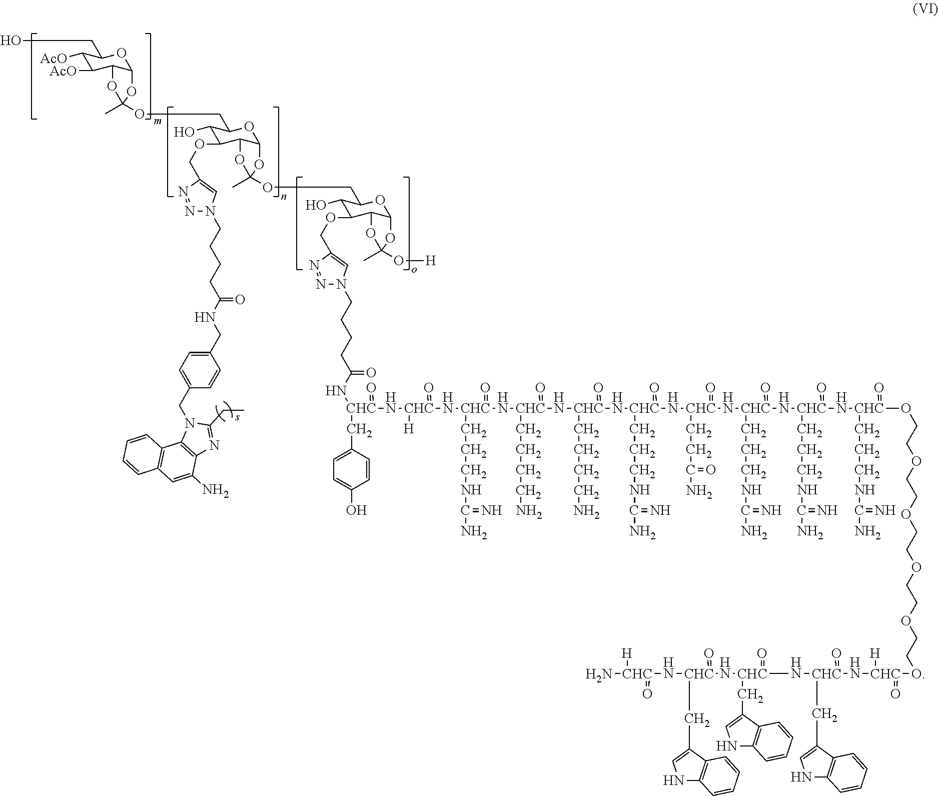

Also disclosed is a nanoparticle comprising one or more polymers disclosed herein, for example a polymer having formula (I), (II), (III), (IV), (V), or (VI).

Further disclosed, in some aspects, is a method of stimulating an immune response to an antigen, the method comprising administering to a subject a pharmaceutical composition comprising the antigen and an effective amount of a polymer disclosed herein and/or a nanoparticle disclosed herein. Also disclosed, in some aspects, is a method of improving an efficacy of a vaccine, the method comprising administering to a subject a pharmaceutical composition comprising the vaccine and an effective amount of a polymer disclosed herein and/or a nanoparticle disclosed herein. Also described, in some aspects, is a method of stimulating activation and/or proliferation of CD8+ T cells in a subject comprising administering to the subject an effective amount of a polymer disclosed herein and/or a nanoparticle disclosed herein. Additionally disclosed, in some aspects, is a method of stimulating CD4+ T cell activation or proliferation in a subject comprising administering to the subject an effective amount of a polymer disclosed herein and/or a nanoparticle disclosed herein. In some aspects, the method further comprises administering an antigen to the subject. In some aspects, the subject or cell is human.

In some aspects, the pharmaceutical composition further comprises an additional adjuvant. In some aspects, the additional adjuvant is a toll-like receptor (TLR) agonist. The TLR agonist may be any TLR agonist recognized in the art or disclosed herein, including, for example, a TLR1, TLR2, TLR3, TLR4, TLR5, TLR6, TLR7, TLR8, and/or TLR9 agonist. In some aspects, the TLR agonist is a TLR4 agonist. In some aspects, the TLR4 agonist is lipopolysaccharide (LPS), monophosphoryl lipid A (MPLA), Fimbriae H protein (FimH), Microfilarial protein (MfP), or a synthetic TLR4 agonist including a phosphorylated hexaacyl disaccharide (PHAD), an aminoalkyl glucosaminide phosphate (AGP), an OMV with attenuated endotoxicity (fmOMV), E6020, or a combination thereof. In some aspects, the TLR agonist is a TLR 7/8 agonist.

Also disclosed herein, in some aspects, is a method for treatment or prevention of cancer, the method comprising administering to a subject an effective amount of a pharmaceutical composition comprising a polymer disclosed herein and/or a nanoparticle disclosed herein. In some aspects, the method further comprises administering an additional cancer therapy to the subject. In some aspects, the additional cancer therapy comprises chemotherapy, radiation therapy, immunotherapy, or a combination thereof. In some aspects, the additional cancer therapy comprises immunotherapy. In some aspects, the additional cancer therapy is a checkpoint inhibitor therapy. In some aspects, the subject has not been diagnosed with cancer. In some aspects, the subject has been diagnosed with cancer. In some aspects, the subject was previously treated for cancer with a previous therapy. In some aspects, the subject was determined to be resistant to the previous therapy. In some aspects, the pharmaceutical composition is administered to the subject intratumorally. In some aspects, the subject or cell is human.

Disclosed herein, in some aspects, is a copolymer comprising:
a) from 10 to 90% by weight of a repeat unit derived from a monomer of formula (VII);

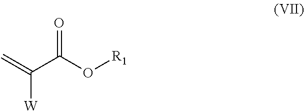

where W is hydrogen or methyl; and
R₁ is an alkyl group having from 1 to 5 carbon atoms, an ether group having an alkyl group of from 1 to 5 carbon atoms between the carboxylate oxygen and the ether oxygen and a methyl or ethyl group attached to the ether oxygen, or an N-terminal alkyl amino group having from 1 to 5 carbon atoms; and
b) from 10 to 90% by weight of a repeat unit derived from a monomer of formula (VIII);

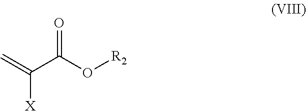

where X is hydrogen or methyl; and
R₂ is an alkyl group having from 1 to 5 carbon atoms, an ether group having an alkyl group of from 1 to 5 carbon atoms between the carboxylate oxygen and the ether oxygen and a methyl or ethyl group attached to the ether oxygen, or an N-terminal alkyl amino group having from 1 to 5 carbon atoms.

In some aspects, wherein R₁ is an N-terminal alkyl amino group having from 1 to 5 carbon atoms, wherein the amino group is substituted with a methyl group, two methyl groups, or a tert-butyloxycarbonyl (Boc) group, or a PEG ether having from 1 to 5 ethylene glycol groups and terminating in a methyl group or ethyl group. In some aspects, R₂ is an N-terminal alkyl amino group having from 1 to 5 carbon atoms, wherein the amino group is substituted with a methyl group, two methyl groups, or a tert-butyloxycarbonyl (Boc) group, or a PEG ether having from 1 to 5 ethylene glycol groups and terminating in a methyl group or ethyl group. In some aspects, R₁ and/or R₂ is an N-terminal alkyl amino group having 2 carbon atoms. In some aspects, R₁ and/or R₂ is an N-terminal alkyl amino group substituted with two methyl groups. In some aspects, R₁ and/or R₂ is an ether group having an alkyl group of 2 carbon atoms between the carboxylate oxygen and the ether oxygen and a methyl group attached to the ether oxygen. In some aspects, R₁ and/or R₂ is an alkyl group having four carbon atoms. In some aspects, R₁ and/or R₂ is an N-terminal alkyl amino group having 2 carbon atoms.

In some aspects, the copolymer further comprises:
c) from 10 to 90% by weight of a repeat unit derived from a monomer of formula (IX);

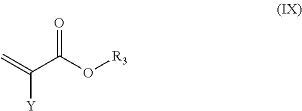

where Y is hydrogen or methyl; and
R₃ is an alkyl group having from 1 to 5 carbon atoms, an ether group having an alkyl group of from 1 to 5 carbon atoms between the carboxylate oxygen and the ether oxygen and a methyl or ethyl group attached to the ether oxygen, an N-terminal alkyl amino group having from 1 to 5 carbon atoms.

In some aspects, R₃ is an N-terminal alkyl amino group having from 1 to 5 carbon atoms, wherein the amino group is substituted with a methyl group, two methyl groups, or a tert-butyloxycarbonyl (Boc) group, or a PEG ether having from 1 to 5 ethylene glycol groups and terminating in a methyl group or ethyl group. In some aspects, R₃ is an N-terminal alkyl amino group having 2 carbon atoms. In some aspects, R₃ is an N-terminal alkyl amino group substituted with two methyl groups. In some aspects, R₃ is an ether group having an alkyl group of 2 carbon atoms between the carboxylate oxygen and the ether oxygen and a methyl group attached to the ether oxygen. In some aspects, R₃ is an alkyl group having four carbon atoms.

In some aspects, the copolymer further comprises:
d) from 10 to 90% by weight of a repeat unit derived from a monomer of formula (X);

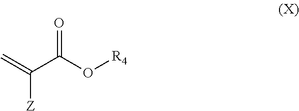

where Z is hydrogen or methyl; and
R₄ is an alkyl group having from 1 to 5 carbon atoms, an ether group having an alkyl group of from 1 to 5 carbon atoms between the carboxylate oxygen and the ether oxygen and a methyl or ethyl group attached to the ether oxygen, an N-terminal alkyl amino group having from 1 to 5 carbon atoms.

In some aspects, R₄ is an N-terminal alkyl amino group having from 1 to 5 carbon atoms, wherein the amino group is substituted with a methyl group, two methyl groups, or a tert-butyloxycarbonyl (Boc) group, or a PEG ether having from 1 to 5 ethylene glycol groups and terminating in a methyl group or ethyl group. In some aspects, R₄ is an N-terminal alkyl amino group having 2 carbon atoms. In some aspects, R₄ is an N-terminal alkyl amino group substituted with two methyl groups. In some aspects, R₄ is an ether group having an alkyl group of 2 carbon atoms between the carboxylate oxygen and the ether oxygen and a methyl group attached to the ether oxygen. In some aspects, R₄ is an alkyl group having four carbon atoms.

In some aspects, the copolymer further comprises at least two end groups. In some aspects, each of the at least two end groups is independently selected from the group consisting of a monomer of formula (VII) to (X), a dithiobenzoyl group, and a 4-cyano-4-yl-pentanoic acid group. In some aspects, the copolymer comprises a number average molecular weight (Mn) ranging from 3,000 to 55,000, including any range or value derivable therein. In some aspects, the copolymer has a dispersity (Ð) ranging from 1.10 to 1.50. In some aspects, the copolymer has Ð ranging from 1.15 to 1.30. In some aspects, the copolymer is a statistical copolymer, a random copolymer, a periodic copolymer, an alternating copolymer, a block copolymer, or a graft copolymer.

Further disclosed herein, in some aspects, is a polymer comprising:
a repeat unit derived from a monomer of formula (VII);

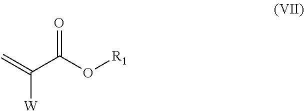

where W is hydrogen or methyl; and
R₁ is an alkyl group having from 1 to 5 carbon atoms, an ether group having an alkyl group of from 1 to 5 carbon atoms between the carboxylate oxygen and the ether oxygen and a methyl or ethyl group attached to the ether oxygen, an N-terminal alkyl amino group having from 1 to 5 carbon atoms.

In some aspects, the amino group is substituted with a methyl group, two methyl groups, or a tert-butyloxycarbonyl (Boc) group, or a PEG ether having from 1 to 5 ethylene glycol groups and terminating in a methyl group or ethyl group. In some aspects, the polymer further comprises at least two end groups. In some aspects, each of the at least two end groups is independently selected from the group consisting of a monomer of formula (VII), a dithiobenzoyl group, and a 4-cyano-4-yl-pentanoic acid group. In some aspects, the polymer comprises a number average molecular weight (Mn) ranging from 3,000 to 55,000, including any range or value derivable therein. In some aspects, the polymer has a dispersity (D) ranging from 1.10 to 1.50. In some aspects, the copolymer comprises Ð ranging from 1.15 to 1.30. In some aspects, the polymer is a linear polymer or a graft polymer.

Also disclosed, in some aspects, is a method for activating an NLRP3 inflammasome in a cell, the method comprising administering to the cell an effective amount of a polymer and/or a nanoparticle of the present disclosure. In some aspects, administering the polymer increases IL-1β production in the cell.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C show a synthetic scheme and proposed mechanism of action of PAI nanovaccines. FIG. 1A depicts a scheme for the synthesis of PAI via sequential click conjugation and for formulation development. FIG. 1B depicts assembly of PAIs into nanostructures via solvent transfer from DMSO to PBS and, where appropriate, electrostatically complexed with antigen. FIG. 1C depicts a proposed mechanism of action of PAI nanovaccines.

FIGS. 2A-2C show results demonstrating ligand ratio optimization. Cytokine secretion in BMDCs at 24 h on incubation with nanoparticles (25 mg/mL). FIG. 2A: TNF-α, FIG. 2B: IL-12p70C, FIG. 2C: IL-1b (n=3, *p<0.05, **p<0.01, ***p<0.001, ****p<0.0001, statistical analysis performed using ANOVA).

FIGS. 3A-3D show results demonstrating that PAI induces inflammasome activation and antigen cross-presentation. FIG. 3A shows caspase-I activity in BMDC following incubation with activators for 18 h. FIGS. 3B and 3C show IL-1b (FIG. 3B) and IL-18 (FIG. 3C) secretion in supernatants following incubation with BMDCs for 18 h. Co-incubation of PAI with NLRP3 inhibitor MCC-950 results in loss of IL-1b and Il-18 activity. FIG. 3D shows flow cytometry analysis of SIINFEKL-H2K^{b} complexes on surface of BMDCs following 24 h incubation of various activators along with OVA antigen (n=3, *p<0.05, **p<0.01, ***p<0.001, ****p<0.0001, statistical analysis performed using ANOVA).

FIGS. 4A-4C show results demonstrating that PAI induces endosomolysis and cytosolic delivery. Various activators were incubated along with DQ Green BSA on THP-1 cells for 12 h and cells were stained with Hoechst and Lysoview 633 followed by confocal microcopy imaging. Representative images shown for PBS (FIG. 4A), unlinked (UL) mixture (FIG. 4B), and PAI (FIG. 4C).

FIGS. 5A-5F shows results demonstrating bio-distribution of PAI-Ova formulations. Ova-AF-647 (FIG. 5A), UL/Ova-AF647 (FIG. 5B), or PAI/Ova-AF647 (FIG. 5C) were injected subcutaneously in the flank of mice and imaged by IVIS at 3 h, 24 h, 48 h and 72 h post-injection. FIGS. 5D-5E show images of distribution of AF-647 in lungs and heart, inguinal lymph one, spleen, liver, and kidney 48 hours post-injection of Ova-AF-647 (FIG. 5D), UL/Ova-AF647 (FIG. 5E), or PAI/Ova-AF647 (FIG. 5F).

FIGS. 6A-6G show results demonstrating that PAI enhances vaccine efficacy. FIGS. 6A-6D show vaccination studies with OVA antigen (20 mg) along with various formulations. FIG. 6A: Study design. Mice (n=5) were injected with vaccines intramuscularly on day 0 followed by a boost on day 14. Antigen-specific response was analyzed on day 24. FIG. 6B: Serum anti-Ova IgG Titer; FIGS. 6C and 6D: Analysis of splenocytes for antigen-specific T-cells; Splenocytes were stimulated ex-vivo with class I or class II OVA epitopes and analyzed for antigen-specific responses following intracellular cytokine staining: percentage of IFN-g secreting CD4+ splenocytes (FIG. 6C) and percentage of IFN-g secreting CD8+ splenocytes (FIG. 6D). FIGS. 6E-6G demonstrate efficacy of OVA vaccine formulations in a EG7.OVA tumor model. FIG. 6E: Study design. Mice (n=6) were inoculated with EG7.OVA cells (2.0× 10⁵) and injected with various vaccine formulations along with OVA on day 5 and day 12. FIG. 6F: Kaplan-Maier survival analysis of mice treated with various formulations. FIG. 6G: Growth curves of tumors until the first mouse died (day 22). (*p<0.05, **p<0.01, ***p<0.001, ****p<0.0001, statistical analysis performed using ANOVA in FIGS. 6B-6D and 6G, and by using log-rank test with Bonferroni-correction in FIG. 6F)

FIGS. 7A-7G show results demonstrating PAI vaccine efficacy in B16.F10 tumor model. FIG. 7A: Study design. Mice (n=7) were inoculated with B16.F10 cells (1.0×10⁵) on day 0. Formulations of PAI or unlinked activators with four B16.F10 antigens were injected peritumorally on day 9 followed by boost vaccinations on day 15. These injections were accompanied with intraperitoneal checkpoint blockade antibody cocktail (anti-CTLA-4+ anti-PDL-1) injections. A parallel study was performed, and mice were sacrificed on day 22 and spleens and tumors were extracted to evaluate antigen-specific cellular response. FIG. 7B: Growth curves of tumors until the first mouse died (day 20); FIG. 7C: Kaplan-Maier survival analysis of mice treated with various formulations. FIGS. 7D-7G: Splenocytes were stimulated ex-vivo with antigen cocktail for 48 h and supernatants were analyzed for cytokines using cytometric bead array FIG. 7D: IFN-g; FIG. 7E: Granzyme-B; FIG. 7F: IL-10; FIG. 7G: Ratio of IFN-g and IL-10 in supernatants (*p<0.05, **p<0.01, ***p<0.001, ****p<0.0001, statistical analysis performed using ANOVA in FIGS. 7B and 7D-7G and by using log-rank test with Bonferroni-correction in FIG. 7C).

FIGS. 8A-8F show IHC analysis of tumor tissues. FIGS. 8A-8C show representative IHC staining of tumor tissues from PBS, ICB and PAI groups on day 22. Tumor infiltrating T-cells (CD4, CD8 and Foxp3) are stained in green and indicated with arrows. FIG. 8A-CD4; FIG. 8B-CD8; FIG. 8C-Foxp3 staining. FIGS. 8D-8F show corresponding quantitative summary data from 8 IHC samples per group. FIG. 8D-CD4; FIG. 8E-CD8; FIG. 8F-Foxp3 staining. Statistical significance is determined relative to PAI/ICB using one-way ANOVA with Dunnett's multiple comparisons test.

FIGS. 9A-9H shows results demonstrating PAI vaccine efficacy in CT-26 tumor model. FIG. 9A: Study design. Mice (n=10) were inoculated with CT-26 cells (2.0×10⁵) on day 0. Formulations of PAI or unlinked activators with four CT-26 antigens were injected peritumorally on day 13 followed by boost vaccinations on day 18 and day 23. These injections were accompanied with intraperitoneal checkpoint blockade antibody cocktail (ICB: CTLA-4+PDL-1) injections. FIG. 9B: Kaplan-Maier survival analysis of mice treated with various formulations. FIG. 9C: Representative animals from PBS group on Day 33; FIG. 9D: Representative animals in PAI formulation treated group on day 49. FIGS. 9E-9G. Tumor growth curves in PBS (FIG. 9E), ICB (FIG. 9F), PAI (FIG. 9G), and PAI no antigen/ICB (FIG. 9H) treated animals. (*p<0.05, **p<0.01, statistical analysis performed using log-rank test with Bonferroni-correction in FIG. 9B).

FIGS. 10A-10F shows results from toxicity analyses. FIG. 10A: Study Design. Mice (n=5) were inoculated with CT-26 cells (2.0×10⁵) on day 0. Formulations of PAI or unlinked activators with four CT-26 antigens were injected peritumorally on day 13 with or without intraperitoneal ICB (anti-CTLA4+ anti-PDL1) injections. Blood was collected two hours post-injection for serum cytokine analysis and two days post-injection for analysis of cellular populations. FIGS. 10B and 10C: Analysis of serum cytokines; TNF-a (FIG. 10B) and IL-6 (FIG. 10C); FIGS. 10D-10F. Blood cell counts 2 days post first injection; WBC (FIG. 10D), Lymphocytes (FIG. 10E) and Thrombocytes (FIG. 10F) (n=5, statistical analysis conducted using ANOVA *p<0.05, **p<0.01, ***p<0.001, ****p<0.0001).

FIG. 11. Compositions targeted for high throughput polymer library. Polymers were varied over three dimensions including molecular weight, hydrophobicity, and charge by using four monomers in variable ratios in a controlled, living polymerization approach.

FIGS. 12A-12D show results from IL-1β ELISA assays on tested polymers. All polymers were tested in triplicate at five concentrations (from left to right within each condition): 100, 50, 25, 12.5, 6.25 μg/mL. FIG. 12A: AEMA-s-TEGMA IL-1β. FIG. 12B: DMAEMA-s-TEGMA IL-1β. FIG. 12C: AEMA-s-BMA IL-1β. FIG. 12D: DMAEMA-s-BMA IL-1β.

FIG. 13 shows IL-1β production incurred by polymers. Polymers were incubated with LPS-primed THP-1 cells at 100, 10, and 1 μg/mL (left-to-right) and IL-1β secretion into the supernatant was assayed by ELISA after 5 h.

FIGS. 14A-14B shows supplementary schemes. FIG. 14A: Synthesis of azide functionalized toll like receptor (7/8) agonist (2BXy-azide) used to prepare PAI. FIG. 14B: Synthesis of sugar poly(orthoester) (SPOE) scaffold 1.

FIG. 15 shows the chemical structure of the azide modified TAT-P6-GWWWG peptide used to prepare PAI.

FIG. 16 is a ¹H-NMR spectrum of 2BXy azide (2).

FIG. 17 is ¹H-NMR spectrum of SPOE monomer I.

FIG. 18 is a ¹³C-NMR spectrum of SPOE monomer I.

FIG. 19 is a ¹H-NMR spectrum of SPOE monomer II.

FIG. 20 is a ¹³C-NMR spectrum of SPOE monomer II.

FIG. 21 is a ¹H-NMR spectrum of the SPOE polymer.

FIG. 22 shows GPC analysis of SPOE, 2BXy-SPOE, and PAI.

FIGS. 23A-23B show HPLC analysis of PAI. FIG. 23A shows a standard concentration/absorbance plot for TAT-P6-GWWWG. FIG. 23B shows a standard concentration/absorbance plot for 2BXy. FIG. 23C shows an HPLC trace of the incubation degradation product of PAI. The two peaks correspond to TAT-P6-GWWWG and 2BXy.

FIG. 24 is a table depicting antibodies that were used in various experiments disclosed herein.

FIG. 25 is a table depicting ratios of TAT7/8 and TAT-P6-GWWWG in the polymer library as determined by HPLC.

FIGS. 26A-26B shows TEM images of PAI with 1.5:1 ratio of 2BXy:TAT-P6-GWWWG (FIG. 26A) and 0:1 ratio of 2BXy:TAT-P6-GWWWG (FIG. 26B, formulation control).

FIG. 27 is a table depicting size characterization (diameters and hydrodynamic radii) of the PAI library molecules.

FIGS. 28A-28B shows neoantigen peptides synthesized for immunotherapy studies. FIG. 28A: A table depicting B16-F10 neoantigen peptides synthesized for immunotherapy studies. FIG. 28B: A table depicting CT26 neoantigen peptides synthesized for immunotherapy studies.

FIGS. 29A-29D shows HPLC characterization of PAI assembled with B16.F10 peptides. FIG. 29A: HPLC trace of antigen cocktail (trace with taller peaks) and un-encapsulated B16.F10 antigen from PAI-antigen formulation (trace with shorter peaks). FIG. 29B: A table depicting encapsulation efficiency of B16.F10 peptides calculated using HPLC traces. FIG. 29C: HPLC trace of antigen cocktail (trace with taller peaks) and un-encapsulated CT26 antigen from PAI-antigen formulation (trace with shorter peaks). FIG. 29D: A table depicting encapsulation efficiency of CT26 peptides.

FIGS. 30A-30E shows cytokine data determined from multiplexed bead analysis used in the PAI vaccine efficacy in B16.F10 tumor model data depicted in FIG. 7. FIG. 30A: IL-6; FIG. 30B: IL-2; FIG. 30C: IL-17; FIG. 30D: IL-4; FIG. 30E TNF-α. Statistical significance is noted relative to PAI/ICB (WT) using one-way ANOVA with Dunnett's multiple comparisons test.

FIGS. 31A-31B shows intracellular IFN-γ secretion by restimulated splenocytes. Splenocytes were stimulated ex-vivo with neoantigen peptide cocktail (5 μg/mL of each peptide). Golgiplug was added following 2 h of stimulation. Cells were treated 4 h longer, then fixed, permeabilized, stained for IFN-γ and T cell lineage markers, and analyzed via flow cytometry. FIG. 31A: IFN-γ of CD8+ splenocytes. FIG. 31B: IFN-γ of CD4⁺ splenocytes. Statistical analyses were performed using one-way ANOVA with Dunnett's multiple comparisons test (determined relative to PAI/ICB WT treatment.)

FIG. 32 shows a Kaplan-Maier survival curve of antigen+ICB treated mice in a CT-26 model. No statistical difference was identified between antigen+ICB and ICB alone.

### DETAILED DESCRIPTION

Disclosed herein, in some aspects, are compositions comprising NLRP3 inflammasome activating peptides, in some cases in combination with adjuvants such as TLR agonists, conjugated to various non-immunogenic polymer scaffolds. Aspects of the present disclosure are directed to use of such compositions as vaccine adjuvants. Further aspects are directed to use of such compositions in cancer treatment such as anti-cancer neo-antigen therapy.

An identical water-soluble and water-insoluble scaffold were tested to determine how altered physico-chemical properties impact the activity of the peptide and can modulate immunogenicity. It was observed that these materials enhanced antigen-specific T-cell activation which was dependent on NLRP3 inflammasome activation. Further, results indicated that while all the formulations initiated a robust CD4+ response, CD8+ activation by the material significantly depended on the mode of presentation of the adjuvant.

Disclosed is a new nano-therapeutic capable of inducing robust NLRP3 inflammasome activation and potent tumor-specific cellular response to neo-antigens. Moreover, in combination with immune checkpoint blockade therapy, the disclosed nano-vaccines resulted in complete tumor remission of established tumors in up to 70% of treated animals in highly aggressive tumor models. Accordingly, aspects of the present disclosure are directed to cancer treatment methods comprising administering an NLRP3 inflammasome activating nanotherapeutic of the present disclosure to a subject, in some cases together with a tumor antigen. The disclosed compositions provide a framework for generation of thousands of modulate, soft-material NLRP3 activators that can be used as T-cell activating immune adjuvants with modulable activity.

The meaning of certain terms as intended is defined herein below.

### I. Definitions

Throughout this application, the term “about” is used to indicate that a value includes the inherent variation of error for the measurement or quantitation method.

As used herein, the term “agonist” refers to a molecule that, in combination with a receptor, can produce a cellular response. An agonist may be a ligand that directly binds to the receptor. Alternatively, an agonist may combine with a receptor indirectly by, for example, (a) forming a complex with another molecule that directly binds to the receptor, or (b) otherwise resulting in the modification of another molecule so that the other molecule directly binds to the receptor. An agonist may be referred to as an agonist of a particular receptor or family of receptors (e.g., a TLR agonist or a TNF/R agonist).

“Individual, “subject,” and “patient” are used interchangeably and can refer to a human or non-human.

The terms “lower,” “reduced,” “reduction,” “decrease,” or “inhibit” are all used herein generally to mean a decrease by a statistically significant amount. However, for avoidance of doubt, “lower,” “reduced,” “reduction, “decrease,” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “alkyl,” by itself or as part of another substituent, means, unless otherwise stated, a linear (i.e. unbranched) or branched carbon chain, which may be fully saturated, mono- or polyunsaturated. An unsaturated alkyl group is one having one or more double bonds or triple bonds. Saturated alkyl groups include those having one or more carbon-carbon double bonds (alkenyl, also olefinic) and those having one or more carbon-carbon triple bonds (alkynyl). The groups, —CH3 (Me), —CH2CH3 (Et), —CH2CH2CH3 (n-Pr), —CH(CH3) 2 (iso-Pr), —CH2CH2CH2CH3 (n-Bu), —CH(CH3) CH2CH3 (sec-butyl), —CH2CH(CH3) 2 (iso-butyl), —C (CH3) 3 (tert-butyl), —CH2C(CH3) 3 (neo-pentyl), are all non-limiting examples of alkyl groups.

The term “aryl” means a polyunsaturated, aromatic, hydrocarbon substituent. Aryl groups can be monocyclic or polycyclic (e.g., 2 to 3 rings that are fused together or linked covalently). The term “heteroaryl” refers to an aryl group that contains one to four heteroatoms selected from N, O, and S. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl) 2amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl) 2amino, alkylsulfinyl, alkylsulfonyl, arylsulfonyl, unsubstituted cycloalkyl, unsubstituted heterocyclyl, unsubstituted aryl, or unsubstituted heteroaryl. Exemplary optional substituents include, but are not limited to: —OH, oxo (═O), —Cl, —F, Br, C1-4alkyl, phenyl, benzyl, —NH2, —NH(C1-4alkyl), —N(C1-4alkyl) 2, —NO2, —S(C1-4alkyl), —SO2 (C1-4alkyl), —CO2 (C1-4alkyl), and—O(C1-4alkyl).

Suitable pharmaceutically acceptable salts may also be formed by reacting the agents of the invention with an organic base such as methylamine, ethylamine, ethanolamine, lysine, ornithine and the like. Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

It should be recognized that the particular anion or cation forming a part of any salt of this invention is not critical, so long as the salt, as a whole, is pharmacologically acceptable.

Additional examples of pharmaceutically acceptable salts and their methods of preparation and use are presented in Handbook of Pharmaceutical Salts: Properties, Selection and Use (2002), which is incorporated herein by reference.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention. With respect to pharmaceutical compositions, the term “consisting essentially of” includes the active ingredients recited, excludes any other active ingredients, but does not exclude any pharmaceutical excipients or other components that are not therapeutically active.

The term “consisting of” refers to compositions, methods, and respective components thereof as described herein, which are exclusive of any element not recited in that description of the embodiment.

As used in this specification and the appended claims, the singular forms “a”, “an”, and “the” include plural references unless the context clearly dictates otherwise. Thus for example, references to “the method” includes one or more methods, and/or steps of the type described herein and/or which will become apparent to those persons skilled in the art upon reading this disclosure and so forth.

### II. Polymers

Aspects of the present disclosure relate to various polymers. Polymers disclosed herein may be useful in targeted delivery of one or more agents, for example, immune modulating agents, to a subject. Examples of polymers useful in the compositions and methods of the present disclosure include N-(2-Hydroxypropyl) methacrylamide (HPMA), poly(orthoester) s, and polysaccharides. The disclosed polymers may include, for example, functionalized polymers. In some aspects, disclosed herein are functionalized poly(orthoester) s comprising one or more polypeptides. In some aspects, a functionalized poly(orthoester) of the present disclosure comprises an inflammasome activator. In some aspects, a polymer comprising an inflammasome activator is described as an inflammasome activating polymer.

In some aspects, the present disclosure provides a polymer having the general formula:

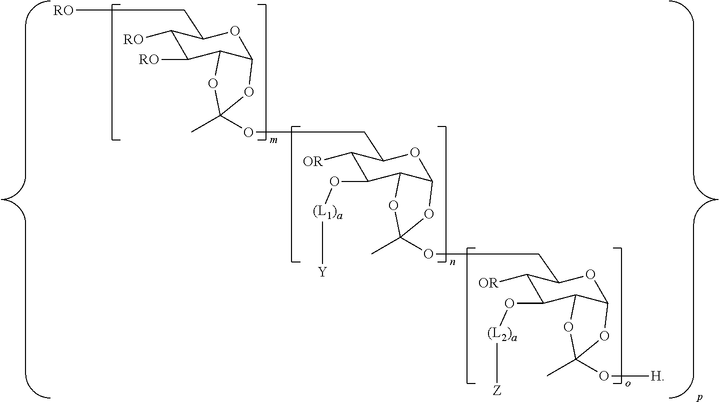

In some aspects, each R is hydrogen. In some aspects, each R is not hydrogen. In some aspects, each R is alkyl. In some aspects, each R is acyl. In some aspects, each R is an acetyl group.

In some aspects, m is an integer ranging from 0 to 10 (or any range or value derivable therein). In some aspects, m is 0, 1, 2, 3, 4, 5, 6, 7, 8, 9, or 10 (or any range or value derivable therein). In some aspects, n is an integer ranging from 0 to 10 (or any range or value derivable therein). In some aspects, n is 0, 1, 2, 3, 4, 5, 6, 7, 8, 9, or 10. In some aspects, o is an integer ranging from 0 to 10 (or any range or value derivable therein). In some aspects, o is 0, 1, 2, 3, 4, 5, 6, 7, 8, 9, or 10 (or any range or value derivable therein). Contemplated are polymers wherein m, n, and o may each independently be any of 0, 1, 2, 3, 4, 5, 6, 7, 8, 9, or 10, including any combination of values of m, n, and o therein. In some aspects, m is 0, n is 0, 1, 2, 3, 4, 5, 6, 7, 8, 9, or 10, and o is 0, 1, 2, 3, 4, 5, 6, 7, 8, 9, or 10. In some aspects, m is 1, n is 0, 1, 2, 3, 4, 5, 6, 7, 8, 9, or 10, and o is 0, 1, 2, 3, 4, 5, 6, 7, 8, 9, or 10. In some aspects, m is 2, n is 0, 1, 2, 3, 4, 5, 6, 7, 8, 9, or 10, and o is 0, 1, 2, 3, 4, 5, 6, 7, 8, 9, or 10. In some aspects, m is 3 n is 0, 1, 2, 3, 4, 5, 6, 7, 8, 9, or 10, and o is 0, 1, 2, 3, 4, 5, 6, 7, 8, 9, or 10. In some aspects, m is 4, n is 0, 1, 2, 3, 4, 5, 6, 7, 8, 9, or 10, and o is 0, 1, 2, 3, 4, 5, 6, 7, 8, 9, or 10. In some aspects, m is 5, n is 0, 1, 2, 3, 4, 5, 6, 7, 8, 9, or 10, and o is 0, 1, 2, 3, 4, 5, 6, 7, 8, 9, or 10. In some aspects, m is 6, n is 0, 1, 2, 3, 4, 5, 6, 7, 8, 9, or 10, and o is 0, 1, 2, 3, 4, 5, 6, 7, 8, 9, or 10. In some aspects, m is 7, n is 0, 1, 2, 3, 4, 5, 6, 7, 8, 9, or 10, and o is 0, 1, 2, 3, 4, 5, 6, 7, 8, 9, or 10. In some aspects, m is 8, n is 0, 1, 2, 3, 4, 5, 6, 7, 8, 9, or 10, and o is 0, 1, 2, 3, 4, 5, 6, 7, 8, 9, or 10. In some aspects, m is 9, n is 0, 1, 2, 3, 4, 5, 6, 7, 8, 9, or 10, and o is 0, 1, 2, 3, 4, 5, 6, 7, 8, 9, or 10. In some aspects, m is 10, n is 0, 1, 2, 3, 4, 5, 6, 7, 8, 9, or 10, and o is 0, 1, 2, 3, 4, 5, 6, 7, 8, 9, or 10. In some aspects, n+o is greater than or equal to 1.

In some aspects, the ratio of m:(n+0) is 1:0, 1:1, 1:2, 1:3, 1:4, 1:5, 1:6, 1:7, 1:8, 1:9, 1:10, 2:1, 3:1, 3:2, 4:1, 4:2, 4:3, 5:1, 5:2, 5:3, 5:4, 6:1, 6:2, 6:3, 6:4, 6:5, 7:1, 7:2, 7:3, 7:4, 7:5, 7:6, 8:1, 8:2, 8:3, 8:4, 8:5, 8:6, 8:7, 9:1, 9:2, 9:3, 9:4, 9:5, 9:6, 9:7, 9:8, 10:1, 10:2, 10:3, 10:4, 10:5, 10:6, 10:7, 10:8, or 10:9. In some aspects, the ratio of m:(n+0) is 0:1, 1:1, 5:1, or 10:1. In some aspects, the ratio of m:(n+0) is 5:1.

In some aspects, L₁ and L₂ are linkers. L₁ and L₂ may be, independently, any chemical linker. In some aspects, L₁ includes a maleimide moiety, a PEG moiety, and/or a triazole moiety. In some aspects, L₁ is a triazole linker. In some aspects, L₂ includes a maleimide moiety, a PEG moiety, and/or a triazole moiety. In some aspects, L₂ is a triazole linker. In some aspects, L₁ is a polyethylene glycol (PEG) linker, a N-(2-hydroxypropyl)-methacrylamide (HPMA) linker, a PEG-methylacrylamide (PEGMA) linker, a succinimide linker, a maleimide linker, a polyamide linker, a polyester linker, or a bifunctional or trifunctional linker comprising a combination of the aforementioned linkers. In some aspects, L₂ is a polyethylene glycol (PEG) linker, a N-(2-hydroxypropyl)-methacrylamide (HPMA) linker, a PEG-methylacrylamide (PEGMA) linker, a succinimide linker, a maleimide linker, a polyamide linker, a polyester linker, or a bifunctional or trifunctional linker comprising a combination of the aforementioned linkers. In some aspects, a linker as disclosed herein comprises c monomeric units, where c is an integer from 2 to 20.

In some aspects, a is 0. In some aspects, a is 1. In some aspects, b is 0. In some aspects, b is 1. In some aspects, a is 0 and b is 1. In some aspects, a is 1 and b is 0. In some aspects, a is 1 and b is 1.

In some aspects, Z comprises a polypeptide, In some aspects, Z is of the formula: A-L₃-B. In some aspects, A is a first polypeptide, L₃ is a linker, and B is a second polypeptide. In some aspects, A is a cell penetrating peptide. In some aspects, B is an endosomal escape peptide. The endosomal escape peptide may be a hydrophobic endosomal escape peptide. The endosomal escape peptide may be a polypeptide having a sequence of an endosomal escape domain of a protein. In some aspects, p is an integer ranging from 1 to 500. In some aspects, p is 1, 2, 3, 4, 5, 6, 7, 8, 9, 10, 15, 20, 30, 40, 50, 100, 200, 300, 400, 500, or any range or value derivable therein.

In some aspects, Y comprises an adjuvant. Y may be or comprise any adjuvant recognized in the art or contemplated herein. In some aspects, Y is a pattern recognition receptor (PRR) agonist.

In some aspects, the disclosed polymers are inflammasome activating polymers. Inflammasome activating polymers may be useful in, for example, stimulating an immune response in an subject, improving the efficacy of a vaccine, and enhancing response to cancer immunotherapy. In some aspects, the inflammasome activating polymer is capable of stimulating an immune response in a subject. In some aspects, the inflammasome activating polymer is capable of stimulating IL-1β production in a subject. In some aspects, the inflammasome activating polymer is capable of stimulating activation and/or proliferation of CD4+ T cells in a subject. In some aspects, the inflammasome activating polymer is capable of stimulating activation and/or proliferation of CD8+ T cells in a subject. In some aspects, the inflammasome activating polymer has the general formula

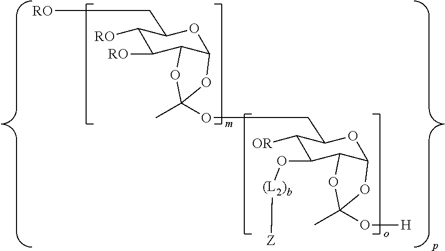

where R is alkyl or acyl, Z is an inflammasome activator, L₂ is a linker, and the inflammasome activating polymer is capable of stimulating activation and/or proliferation of CD8+ T cells in a subject. In some aspects, the subject or cell is human.

In some aspects, the polymer is of formula

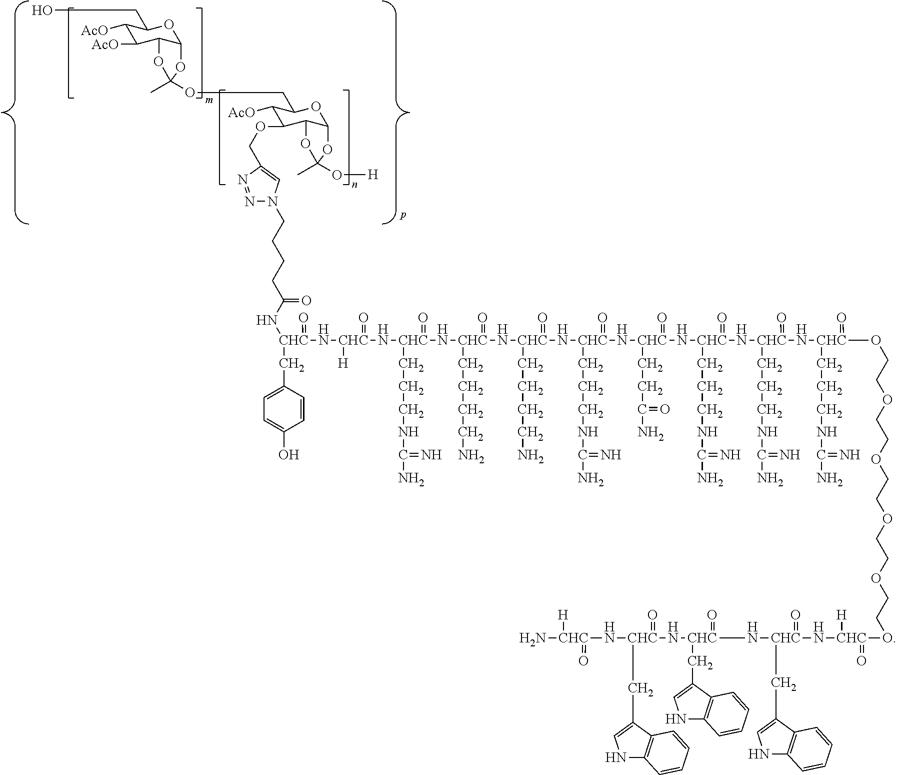

In this embodiment, the polymer is capable of stimulating activation and/or proliferation of CD4+ T cells in a subject. In some aspects, the polymer is of formula

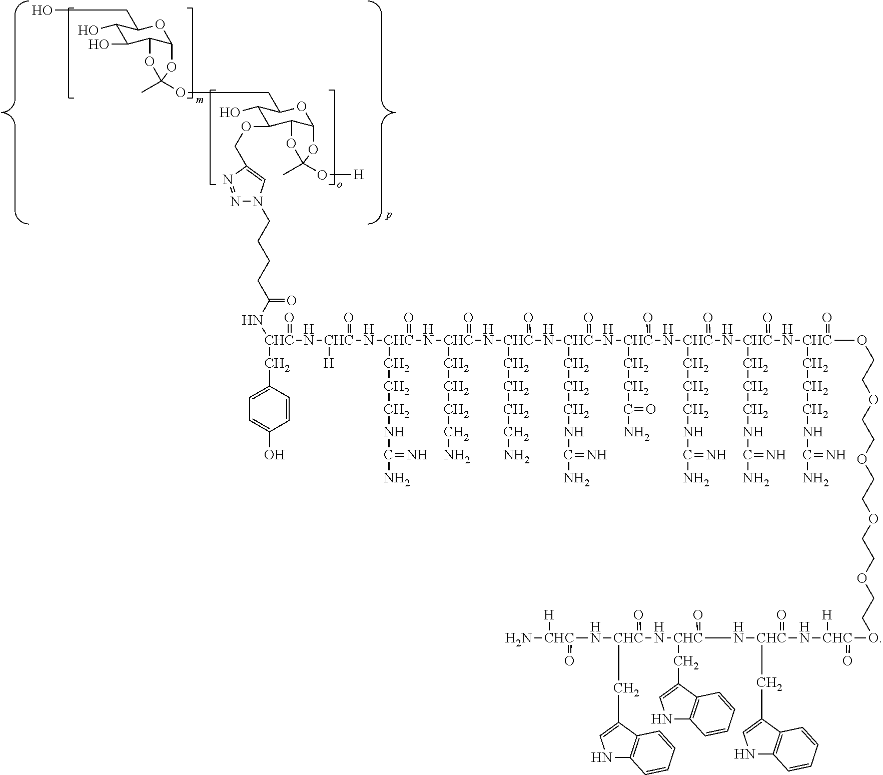

In this embodiment, the polymer is capable of stimulating activation and/or proliferation of CD4+ T cells and CD8+ T cells in a subject.

In some aspects, the polymer is of formula

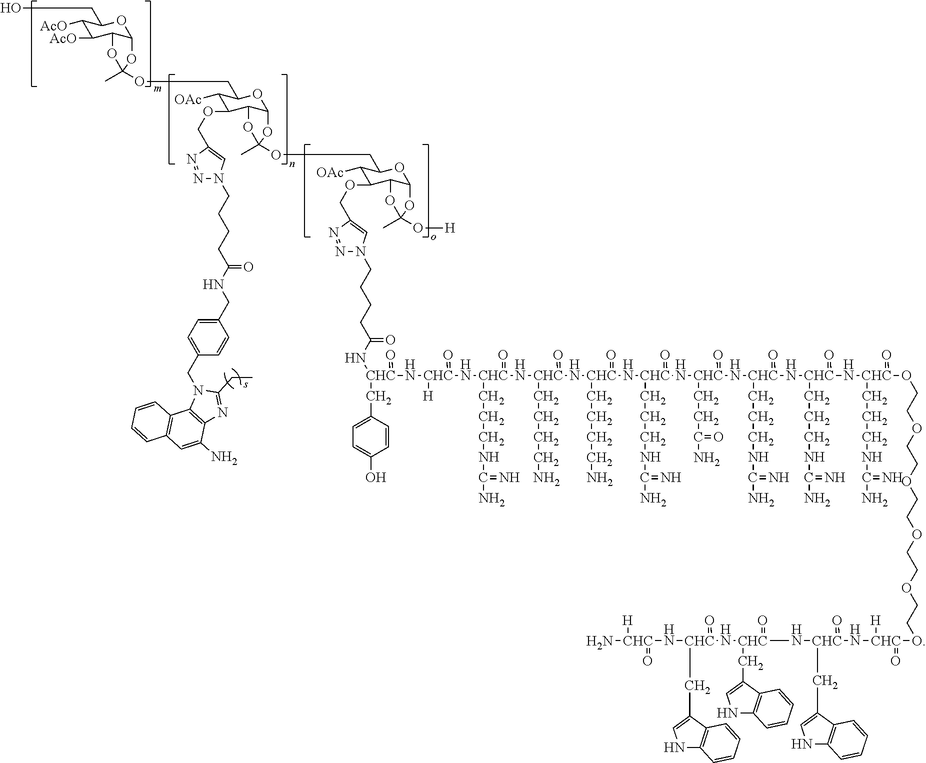

In some aspects, the polymer is of formula

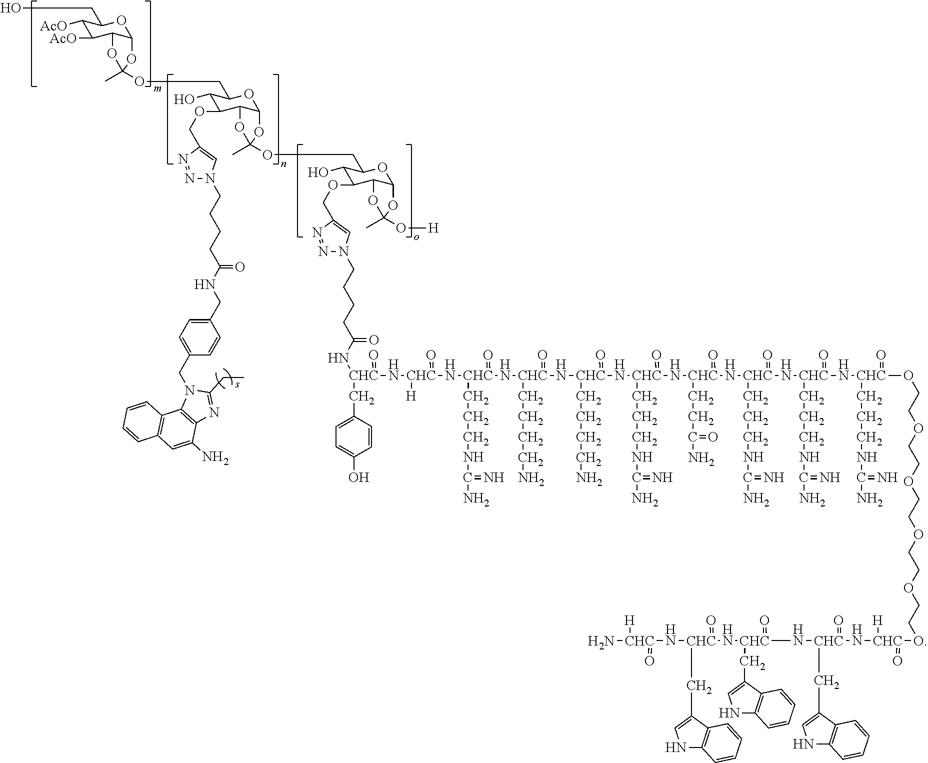

As described herein, certain polymers of the disclosure are capable of self-assembly into nanoparticles. Accordingly, aspects of the disclosure are directed to nanoparticles (e.g., nano micelles) comprising a polymer disclosed herein, for example a polymer having formula (I), (II), (III), (IV), (V), or (VI).

### III. Inflammasome Activators

Aspects of the present disclosure are directed to inflammasome activators. The term “inflammasome activator” describes a molecule or composition capable of stimulating activation of inflammasome activity, in vivo and/or in vitro. Inflammasome activators may be useful in, for example, stimulation of an immune response, improvement of vaccine efficacy, and improvement of anti-cancer immunotherapy. In some aspects, disclosed herein are molecules and compositions comprising inflammasome activating peptides. In some aspects, an inflammasome activator disclosed herein is capable of activating an NLRP3 inflammasome.

In some aspects, an inflammasome activator is an inorganic composition. Examples of inorganic inflammasome activators include alum and silica. In some aspects, an inflammasome activator is an organic composition. Examples of organic inflammasome activators include ATP, pore-forming toxins (e.g., maitotoxin), urea, chemotherapeutic agents (e.g., 5-fluorauracil), and peptides. In some aspects, an inflammasome activator is an inflammasome activating peptide. In some aspects, an inflammasome activating peptide is a natural peptide. In some aspects, an inflammasome activating peptide is a synthetic peptide. An inflammasome activating peptide may be a small peptide (e.g., less than 5 amino acids in length). A non-limiting example of a small inflammasome activating peptide is Leucine-Leucine-O-Methylester.

In some aspects, an inflammasome activating peptide comprises two or more distinct moieties, components, or peptide regions. In some aspects, the inflammasome activating peptide comprises a cell penetrating peptide. Non-liming examples of cell penetrating peptides that may be used in the compositions and methods of the present disclosure include a TAT peptide, penetratin, transportan, MAP, Pep-1, Pept 1, Pept 2, IVV-14, pVEC, HRSV, and polyarginine. In some aspects, the cell penetrating peptide comprises a sequence from an HIV TAT protein. In some aspects, the HIV is HIV-1 or HIV-2. In some aspects, the HIV is HIV-1. In some aspects, the cell penetrating peptide comprises a sequence from amino acids 45-65 of HIV-1 TAT protein. In some aspects, the sequence is SEQ ID NO:1. In some aspects, the sequence is SEQ ID NO:2.

In some aspects, the inflammasome activating peptide comprises an endosomal escape peptide (e.g., a peptide having the sequence of an endosomal escape domain from a protein). In some aspects, the endosomal escape peptide is a hydrophobic endosomal escape peptide. In some aspects, the endosomal escape peptide has the sequence GWWWG (SEQ ID NO: 3). In some aspects, the endosomal escape peptide has the sequence GFWFG (SEQ ID NO: 4). In some aspects, the endosomal escape peptide has the sequence GWWG (SEQ ID NO: 5).

In some aspects, an inflammasome activating peptide comprises a cell penetrating peptide and an endosomal escape peptide, which in some cases are attached by a linker. A linker may be any chemical linker capable of separating the cell penetrating peptide from the endosomal escape peptide while retaining the functional capabilities of each region. In some aspects, the linker is a polyethylene glycol (PEG) linker. In some aspects, the PEG linker is of sufficient length to separate the cell penetrating peptide from the endosomal escape peptide. In some aspects, the PEG linker comprises 2, 3, 4, 5, 6, 7, 8, 9, 10, 11, 12, 13, 14, 15, 16, 17, 18, 19, or 20 ethyleneoxy units, or any range derivable therein. In some aspects, the PEG linker comprises between 4 and 8 ethyleneoxy units. In some aspects, the PEG linker comprises 6 ethyleneoxy units. In some aspects, the linker is a polyethylene glycol (PEG) linker, a N-(2-hydroxypropyl)-methacrylamide (HPMA) linker, a PEG-methylacrylamide (PEGMA) linker, a succinimide linker, a maleimide linker, a polyamide linker, a polyester linker, or a bifunctional or trifunctional linker comprising a combination of the aforementioned linkers. In some aspects, the inflammasome activator of the disclosure is TAT-peg6-GWWWG. “TAT-peg6-GWWWG” (also “TAT-GWWWG peptide”) describes a molecule having formula

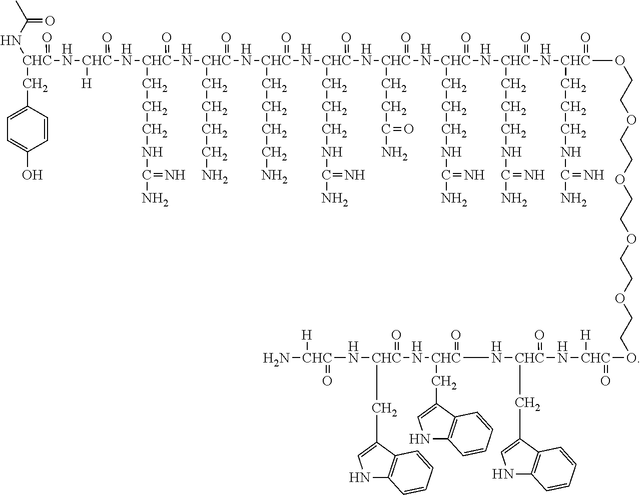

Also described are functionalized inflammasome activators. In some aspects, a functionalized inflammasome activator of the disclosure is an azido-functionalized TAT-GWWWG peptide (N3-TAT-P6-GWWWG).

In some aspects, an inflammasome activator is conjugated to a polymer described herein. A polymer conjugated to an inflammasome activator may be described as an inflammasome activating polymer. In some aspects, a polymer is a poly(orthoester). Conjugation of an inflammasome activator to a polymer may be useful in, for example, targeted delivery of an inflammasome activator, limiting diffusion of an inflammasome activator in a subject, and/or improving an immune response to an inflammasome activator.

### IV. Adjuvants

Aspects of the present disclosure comprise vaccine adjuvants and their use. The term “adjuvant” describes a molecule or composition capable of activating or otherwise modulating an immune response in a subject. An adjuvant may improve an efficacy of a vaccine by improving the immune response to an antigen. Additionally, an adjuvant may improve efficacy of a cancer immunotherapy by enhancing the immune response to a tumor antigen. Compositions of the present disclosure may comprise an antigen and one or more adjuvants. In some aspects, a vaccine composition comprises an antigen and 1, 2, 3, 4, 5, 6, 7, 8, 9, or 10 adjuvants. Certain aspects of the disclosure are based, at least in part, on the discovery of that certain disclosed inflammasome activating peptides and inflammasome activating polymers are useful as adjuvants, either alone or in combination with one or more additional adjuvants. Adjuvants may be provided freely in solution and/or conjugated to a polymer.

Methods of the present disclosure include methods for improving efficacy of a vaccine. Improving efficacy of a vaccine may comprise administering a pharmaceutical composition comprising the vaccine and effective amount of an inflammasome activating peptide disclosed herein. Additional methods include methods for improving efficacy of a cancer immunotherapy (e.g., immune checkpoint blockade therapy). Improving efficacy of a cancer immunotherapy may comprise administering one or more pharmaceutical compositions comprising a cancer immunotherapeutic (e.g., an immune checkpoint inhibitor) and an inflammasome activating peptide disclosed herein. In some aspects, the inflammasome activating peptide is conjugated to a polymer, such as a poly(orthoester) disclosed herein, in some cases in combination with a TLR agonist such as a TLR7/8 agonist or a TLR4 agonist.

In some aspects, a pharmaceutical composition comprises a copolymer comprising an inflammasome activating peptide and one or more additional adjuvants. In some aspects, an adjuvant of the disclosure is a toll-like receptor (TLR) agonist. In some aspects, the TLR agonist is one known in the art and/or described herein. The TLR agonists may include an agonist to TLR1 (e.g., peptidoglycan or triacyl lipoproteins), TLR2 (e.g., lipoteichoic acid; peptidoglycan from Bacillus subtilis, E. coli 0111: B4, Escherichia coli K12, Wolbachia bacteria, or Staphylococcus aureus; atypical lipopolysaccharide (LPS) such as Leptospirosis LPS and Porphyromonas gingivalis LPS; a synthetic diacylated lipoprotein such as FSL-1 or Pam2CSK4; lipoarabinomannan or lipomannan from M. smegmatis; triacylated lipoproteins such as Pam3CSK4; lipoproteins such as MALP-2 and MALP-404 from mycoplasma; Borrelia burgdorferi OspA; Porin from Neisseria meningitidis or Haemophilus influenza; Propionibacterium acnes antigen mixtures; Yersinia LcrV; lipomannan from Mycobacterium or Mycobacterium tuberculosis; helminths, including ringed or segmented worms, thorny-headed worms, flatworms, and roundworms such as Ascaris lumbricoides, Wuchereria bancrofti, and Trichinella; Trypanosoma cruzi GPI anchor; Schistosoma mansoni lysophosphatidylserine; Leishmania major lipophosphoglycan (LPG); Plasmodium falciparum glycophosphatidylinositol (GPI); zymosan; antigen mixtures from Aspergillus fumigatus or Candida albicans; and measles hemagglutinin), TLR3 (e.g., double-stranded RNA, polyadenylic-polyuridylic acid (Poly(A:U)); polyinosine-polycytidylic acid (Poly(I:C)); polyinosine-polycytidylic acid high molecular weight (Poly(I:C) HMW); and polyinosine-polycytidylic acid low molecular weight (Poly(I:C) LMW)), TLR4 (e.g., LPS from Escherichia coli and Salmonella species); TLR5 (e.g., Flagellin from B. subtilis, P. aeruginosa, or S. typhimurium), TLR8 (e.g., single stranded RNAs such as ssRNA with 6UUAU repeats, RNA homopolymer (ssPolyU naked), HIV-1 LTR-derived ssRNA (ssRNA40), or ssRNA with 2 GUCCUUCAA repeats (ssRNA-DR)), TLR7 (e.g., imidazoquinoline compound imiquimod, Imiquimod VacciGrade™ Gardiquimod VacciGrade™, or Gardiquimod™; adenine analog CL264; base analog CL307; guanosine analog loxoribine; TLR7/8 (e.g., thiazoquinoline compound CL075; imidazoquinoline compound CL097, 2Bxy, R₈₄₈, or R₈₄₈ VacciGrade™), TLR9 (e.g., CpG ODNs); and TLR11 (e.g., Toxoplasma gondii Profilin). In some aspects, the TLR agonist is a TLR7 agonist. In some aspects, the TLR agonist is a TLR7/8 agonist. In some aspects, the TLR agonist is 2Bxy. “2Bxy” describes a compound having formula

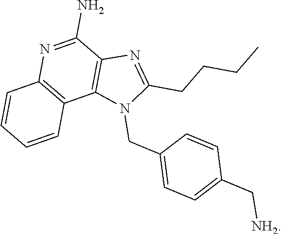 [SMILES: CCCCc1nc2c(N)nc3ccccc3c2n1Cc1ccc(CN)cc1]

Aspects of the disclosure comprise a functionalized TLR agonist, for example a functionalized 2Bxy. An example functionalized 2Bxy of the disclosure is an azido-functionalized 2Bxy. One example azido-functionalized 2Bxy is a molecule having formula

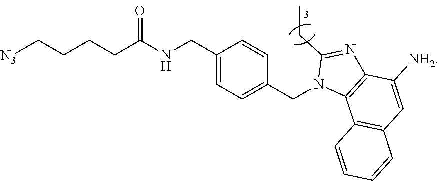

Additional adjuvants contemplated herein include, for example, those described in Hu H G, Li Y M. Front Chem. 2020 Jul. 30; 8:601, incorporated herein by reference in its entirety.

### V. Pharmaceutical Compositions

The present disclosure includes methods for treating or preventing disease and activating immune responses in a subject in need thereof. Aspects are directed to vaccine adjuvants, such as the NLRP3 inflammasome activating peptides and polymers described herein, that may be in the form of a pharmaceutical composition that can be used to induce or modify an immune response to improve the efficacy of a vaccine.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2-day to 12-week intervals, more usually from one to two week intervals. The course of the administrations may be followed by assays for alloreactive immune responses and T cell activity.

Pharmaceutical compositions of the present disclosure may be suitable for use in vaccination. Pharmaceutical compositions may comprise an antigen, an inflammasome activator (e.g., inflammasome activating peptide, which may be attached to a polymer scaffold), and, in some cases, one or more additional adjuvants. Adjuvants may improve an efficacy of a vaccine by improving the immune response to an antigen. A pharmaceutical composition may comprise at least 1, 2, 3, 4, 5, 6, 7, 8, 9, or 10 adjuvants, or any range derivable therein. In some aspects, disclosed herein are pharmaceutical compositions comprising an antigen and an NLRP3 activating peptide of the present disclosure. An NLRP3 activating peptide may be conjugated to a polymer scaffold, as described herein. In some aspects, a pharmaceutical composition comprises an additional adjuvant, such as a TLR agonist. In some aspects, a pharmaceutical composition comprises a pharmaceutically acceptable carrier or excipient.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

### VI. Methods of Making Vaccines

Disclosed herein, in some aspects, are methods for making vaccines comprising generating a pharmaceutical composition comprises an antigen and one or more antigens described herein. In some aspects, a method for making a vaccine comprises generating a pharmaceutical composition comprising an antigen (e.g., a viral antigen, a bacterial antigen, etc.) and an inflammasome activating peptide of the present disclosure (e.g., an inflammasome activating peptide comprising a cell penetrating peptide linked to an endosomal escape domain). In some aspects, a method for making a vaccine comprises generating a pharmaceutical composition comprising an antigen (e.g., a viral antigen, a bacterial antigen, a parasitic antigen, etc.) and an inflammasome activating polymer of the present disclosure (e.g., a glucose poly(orthoester) conjugated to an inflammasome activating peptide). Making a vaccine may comprise addition of one or more additional adjuvants (e.g., TLR agonists) to the pharmaceutical composition.

A vaccine may be formulated in a pharmaceutically acceptable carrier or excipient. For example, a vaccine may be formulated in an oil-in-water nano-emulsion. Examples of nano-emulsions useful in vaccine formulations of the present disclosure include squalene-based emulsions (e.g., Addavax®, MF59®) and paraffin-based emulsions (e.g., incomplete Freund's adjuvant, Complete Freund's adjuvant).

### VII. Methods of Treatment

As discussed above, the compositions and methods of using these compositions can treat a subject (e.g., prevent an infection, evoke a robust immune response to an antigen, or reduce or prevent tumor proliferation) having, suspected of having, or at risk of developing an infection, cancer, or related disease.

As used herein the phrase “immune response” or its equivalent “immunological response” refers to a humoral (antibody mediated), cellular (mediated by antigen-specific T cells or their secretion products) or both humoral and cellular response directed against a protein, peptide, polypeptide, or other antigenic composition of the disclosure in a recipient subject. Treatment or therapy can be an active immune response induced by administration of, e.g., immunogen or a passive therapy effected by administration of antibody, antibody containing material, or primed T-cells.

The presence of a cell-mediated immunological response can be determined by proliferation assays (CD4+ T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject. As used herein and in the claims, the terms “antibody” or “immunoglobulin” are used interchangeably.

In one embodiment a method includes treatment for or prevention of a disease or condition caused by a pathogen. Furthermore, in some examples, treatment comprises administration of other agents commonly used against viral infection, such as one or more antiviral or antiretroviral compounds.

The therapeutic compositions are administered in a manner compatible with the dosage formulation, and in such amount as will be therapeutically effective. The quantity to be administered depends on the subject to be treated. Precise amounts of active ingredient required to be administered depend on the judgment of the practitioner. Suitable regimes for initial administration and boosters are also variable, but are typified by an initial administration followed by subsequent administrations.

The manner of application may be varied widely. Any of the conventional methods for administration of a polypeptide therapeutic are applicable. These are believed to include oral application on a solid physiologically acceptable base or in a physiologically acceptable dispersion, parenterally, by injection and the like. The dosage of the composition will depend on the route of administration and will vary according to the size and health of the subject.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

In certain aspects, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 445, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 micrograms, mg, μg/kg, or mg/kg (or any range derivable therein), of polymeric immunomodulator or other composition.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of a condition. In some aspects, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of an the condition have disappeared or been reduced or after 6, 12, 18, or 24 hours or 1, 2, 3, 4, or 5 days after symptoms of an infection have disappeared or been reduced.

### VIII. Cancer Therapy

In some aspects, the disclosed methods comprise administering a cancer therapy to a subject or patient. The cancer therapy may be chosen based on an expression level measurement, alone or in combination with a clinical risk score calculated for the subject. The cancer therapy may be chosen based on a genotype of a subject. The cancer therapy may be chosen based on the presence or absence of one or more polymorphisms in a subject. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a system cancer therapy. In some aspects, the cancer therapy comprises an immunotherapy, which may be a checkpoint inhibitor therapy. Any of these cancer therapies may also be excluded. Combinations of these therapies may also be administered.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some aspects, the cancer is a Stage I cancer. In some aspects, the cancer is a Stage II cancer. In some aspects, the cancer is a Stage III cancer. In some aspects, the cancer is a Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

### A. Radiotherapy

In some aspects, a radiotherapy, such as ionizing radiation, is administered to a subject. As used herein, “ionizing radiation” means radiation comprising particles or photons that have sufficient energy or can produce sufficient energy via nuclear interactions to produce ionization (gain or loss of electrons). A non-limiting example of ionizing radiation is x-radiation. Means for delivering x-radiation to a target tissue or cell are well known in the art.

In some aspects, the radiotherapy can comprise external radiotherapy, internal radiotherapy, radioimmunotherapy, or intraoperative radiation therapy (IORT). In some aspects, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some aspects, the internal radiotherapy comprises interstitial brachytherapy, intracavitary brachytherapy, or intraluminal radiation therapy. In some aspects, the radiotherapy is administered to a primary tumor.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of radiotherapy administered to a subject may be presented as a total dose of radiotherapy, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of radiation is at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### B. Cancer Immunotherapy

In some aspects, the methods comprise administration of a cancer immunotherapy. Cancer immunotherapy (sometimes called immuno-oncology, abbreviated IO) is the use of the immune system to treat cancer. Immunotherapies can, in some cases, be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and certain examples are described below.

### 1. Checkpoint Inhibitors and Combination Treatment

Aspects of the disclosure may include administration of immune checkpoint inhibitors, examples of which are further described below. As disclosed herein, “checkpoint inhibitor therapy” (also “immune checkpoint blockade therapy,” “checkpoint blockade therapy,” “immune checkpoint therapy,” “ICT,” “checkpoint blockade immunotherapy,” or “CBI”), refers to cancer therapy comprising providing one or more immune checkpoint inhibitors to a subject having or suspected of having cancer.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the V_{H} region of nivolumab, pembrolizumab, or pidilizumab, and the CDR1, CDR2 and CDR3 domains of the V_{L} region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4 or CTLA4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the V_{H} region of tremelimumab or ipilimumab, and the CDR1, CDR2 and CDR3 domains of the V_{L} region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some aspects, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or V_{H} and/or V_{L} domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L₇BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4 (Supp. 1): P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some aspects, the inhibitor comprises the heavy and light chain CDRs or VRs of an anti-LAG3 antibody. Accordingly, in one aspect, the inhibitor comprises the CDR1, CDR2, and CDR3 domains of the V_{H} region of an anti-LAG3 antibody, and the CDR1, CDR2 and CDR3 domains of the V_{L} region of an anti-LAG3 antibody. In another aspect, the antibody has at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4⁺ Th1 and CD8⁺ Tcl cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4⁺ Th1-specific cell surface protein that regulates macrophage activation. Inhibitors of the disclosure may block one or more functions of TIM-3 activity.

In some aspects, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

In some aspects, the inhibitor comprises the heavy and light chain CDRs or VRs of an anti-TIM-3 antibody. Accordingly, in one aspect, the inhibitor comprises the CDR1, CDR2, and CDR3 domains of the V_{H} region of an anti-TIM-3 antibody, and the CDR1, CDR2 and CDR3 domains of the V_{L} region of an anti-TIM-3 antibody. In another aspect, the antibody has at least about 70, 75, 80, 85, 90, 95, 97, or 99% (or any derivable range or value therein) variable region amino acid sequence identity with the above-mentioned antibodies.

### 2. Activator of Co-Stimulatory Molecules

In some aspects, the immunotherapy comprises an activator (also “agonist”) of a co-stimulatory molecule. In some aspects, the agonist comprises an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include activating antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T and CAR-NK Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell, natural killer (NK) cell, or other immune cell. The receptors are called chimeric because they are fused of parts from different sources. CAR-T cell therapy refers to a treatment that uses such transformed cells for cancer therapy, where the transformed cells are T cells. Similar therapies include, for example, CAR-NK cell therapy, which uses transformed NK cells.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

### Example CAR-T Therapies Include Tisagenlecleucel (Kymriah) and Axicabtagene Ciloleucel (Yescarta)

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive Cell Therapy

Adoptive cell therapy is a form of passive immunization by the transfusion of immune cells, such as T cells, NK cells, or other immune cells (also called “adoptive cell transfer”). Immune cells used for adoptive cell therapy include those found in normal tissue and those found in tumor tissue (where they are known as tumor infiltrating immune cells or tumor infiltrating lymphocytes). Although tumor infiltrating immune cells can attack a tumor, the environment within the tumor is generally highly immunosuppressive, preventing immune-mediated tumor death.

Multiple ways of producing and obtaining tumor targeted immune cells have been developed. Immune cells specific to a tumor antigen can be removed from a tumor sample or filtered from blood. Subsequent activation and culturing may be performed ex vivo, with the results reinfused. Activation can take place through gene therapy, by exposing the immune cells to tumor antigens, or by other methods known in the art.

### 7. Oncolytic Virus

In some aspects, the cancer therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

### C. Chemotherapies

In some aspects, a therapy of the present disclosure comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection.

Other suitable chemotherapeutic agents include antimicrotubule agents, e.g., Paclitaxel (“Taxol”) and doxorubicin hydrochloride (“doxorubicin”). Doxorubicin is absorbed poorly and is preferably administered intravenously. In certain aspects, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred in certain cases. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m². Further, 5-FU dosing schedules may be for a variety of time periods, for example up to six weeks, or as determined by one of ordinary skill in the art to which this disclosure pertains.

The amount of the chemotherapeutic agent delivered to a patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### D. Hormone Therapy

In some aspects, a cancer therapy of the present disclosure is a hormone therapy. In particular aspects, a prostate cancer therapy comprises hormone therapy. Various hormone therapies are known in the art and contemplated herein. Examples of hormone therapies include, but are not limited to, luteinizing hormone-releasing hormone (LHRH) analogs, LHRH antagonists, androgen receptor antagonists, and androgen synthesis inhibitors.

### E. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### F. Additional Cancer Therapies

Therapeutic methods disclosed herein may comprise one or more additional cancer therapies. A cancer therapy of the disclosure may comprise, for example, cryoablative therapy, high-intensity ultrasound (also “high-intensity focused ultrasound”), photodynamic therapy, laser ablation, and/or irreversible electroporation. A cancer therapy of the disclosure may comprise 1, 2, 3, 4, 5, or more distinct therapeutic methods.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### IX. Administration of Therapeutic Combinations

The therapy provided herein may comprise administration of a combination of therapeutic agents, such as an immunomodulator (e.g., a polymer of the disclosure, such as a polymer having one of formula (I)-(VI)) and an immunotherapy (e.g., an immune checkpoint blockade therapy). The therapies may be administered in any suitable manner known in the art. For example, the immunomodulator and the immunotherapy may be administered sequentially (at different times) or concurrently (at the same time). In some aspects, the immunomodulator and the immunotherapy are administered in a separate composition. In some aspects, the immunomodulator and the immunotherapy are in the same composition.

In some aspects, the immunomodulator and the immunotherapy are administered substantially simultaneously. In some aspects, the immunomodulator and the immunotherapy are administered sequentially. In some aspects, the immunomodulator or, the immunotherapy, and an additional cancer therapy (e.g., chemotherapy) are administered sequentially. In some aspects, the immunomodulator is administered before administering the immunotherapy. In some aspects, the immunomodulator is administered after administering the immunotherapy.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. For example, one therapeutic agent (e.g., a polymer of the disclosure, such as a polymer having one of formula (I)-(VI)) may be administered by one route of administration (e.g., orally) while a second therapeutic agent (e.g., an immune checkpoint inhibitor) may be administered by a different route of administration (e.g., intraveneously). In some aspects, the immunomodulator is administered intravenously, intramuscularly, subcutaneously, topically, orally, transdermally, intraperitoneally, intraorbitally, by implantation, by inhalation, intrathecally, intraventricularly, or intranasally. In some aspects, the immune checkpoint inhibitor is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

### EXAMPLES

The following examples are included to demonstrate preferred aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

### Example 1—General Procedures and Molecular Characterization

### Mice and Materials

All reactions were conducted under dried nitrogen or argon stream. Anhydrous solvents were purchased in capped DriSolv™ bottles, used without further purification, and stored under argon. Azidohexanoic acid was purchased from Click Chemistry Tools, and PEG linkers were purchased from Quanta Biodesign. All other solvents and reagents were obtained from Sigma Aldrich or Thermo Fisher and used without further purification unless otherwise noted. All glassware was flame-dried before use. Silica gel column chromatography was performed using flash silica gel (32-63 μm). All cell culture reagents unless otherwise noted were purchased from Thermo Fisher. THP-1, B16.F10, and E.G7-OVA cells were purchased from ATCC. Antibodies used for flow cytometry, immunohistochemistry, and checkpoint blockade are listed with clone and vendor in the Supplementary Information. Female C57Bl/6 J, Nu/J, B6.129S6-Nlrp3tm1Bhk/J, and Balb/C mice (5-week-old) were purchased from Jackson Laboratory (JAX). Mice were housed in an AAALAC accredited animal facility. All animal procedures were performed under a protocol approved by the University of Chicago Institutional Animal Care and Use Committee (IACUC). All compounds used in-vivo were tested for endotoxin prior to use. The animals were allowed to acclimatize for at least 7 days prior to experiment onset. All in-vivo experiments were conducted at least two times, and mice were randomly assigned to groups to minimize cage effects. All data unless otherwise noted are analyzed and plotted in GraphPad Prism 9.

### Synthesis of Polymeric Activators of Inflammasomes (PAIs)

The synthesis of PAI was performed by sequential Cu(I) catalyzed Huisgen cycloaddition reaction with the SPOE scaffold. In a representative example, alkyne-containing SPOE (0.037 g, Mn GPC=7.2 kDa, 0.021 mmol alkynes) and 2BXy-N3 (0.006 g, 0.013 mmol) were added to a 10 mL, flame-dried Schlenk flask in anhydrous THF (3.0 mL). After three cycles of freeze-pump-thaw, Cu(I) Br (1.0 mg, 0.0063 mmol) and N,N,N′, N′,N″-pentamethyldiethylenetriamine (4.73 mg, 0.0273 mmol) were added. The reaction was stirred at 37° C. for 12 h. The reaction mixture was purified by passing through neutral alumina and then further precipitated in diethyl ether (3×10 mL) to afford the product (2Bxy-SPOE) as a very light brown powder (0.035 g, 80%). 2Bxy-SPOE was then reacted with azido-TAT-P6-GWWWG (0.023 mg, 0.008 mmol in anhydrous DMF (3.0 mL). After three cycles of freeze-pump-thaw, CuBr (1.0 mg, 0.0063 mmol) and N,N,N′,N′,N″-pentamethyldiethylenetriamine (4.73 mg, 0.0273 mmol) were added. The reaction was stirred at 37° C. for 12 h following which the reaction mixture was dialyzed in EDTA solution followed by dialysis in deionized water. The solution was lyophilized to obtain a brown powder. PAIs with varied ratios of 2BXy:TAT-P6-GWWWG were prepared analogously by varying the molar ratios of these components.

### Nanoassembly of PAIs

PAIs were dissolved in DMSO (1.5 mL) and stirred at room temperature overnight. The solution volume was then subjected to dialysis against endotoxin-free PBS for 24 h to afford NP solution. Following this the particles were stored at 4° C. The stability of the nanoparticles was monitored using TEM over a period of eight weeks at 4° C.

### Characterization of PAIs

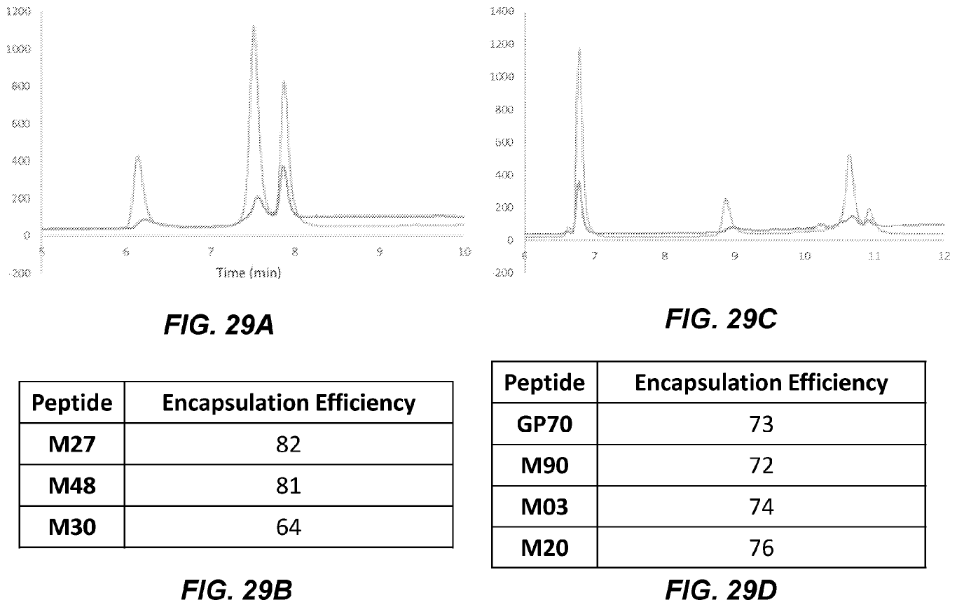

Prior to nanoassembly, PAIs were characterized via gel permeation chromatography (GPC) and high-performance liquid chromatography (HPLC). Following nanoassembly, PAIs were further characterized via transmission electron microscopy (TEM) and dynamic light scattering (DLS) as described in the Supplementary Information. When loaded with neoantigen peptides, encapsulation efficiency of PAIs was again analyzed via HPLC (FIGS. 29A-29D).

### Evaluation of Cytokines Using BMDCs

Bone marrow-derived dendritic cells (BMDCs) were harvested from 6-week-old C57BL/6 J mice (Jackson Laboratory). On day 6, BMDCs were released and plated on 96-well plates at a density of 1.1×10⁶ cells/mL (180 μL) in RPMI+10% heat-inactivated fetal bovine serum (HI-FBS) and incubated with PAI (25 μg/mL) or equivalent quantities of unlinked activators for 18 h following which the plates were centrifuged at 400×G and the supernatants were collected. For studies with MCC-950, cells were pre-incubated with MCC-950 (1 mM) 30 min prior to addition of PAI. IL-1β and IL-18 cytokines were measured in undiluted serum by ELISA (BioLegend ELISA MAX Deluxe kit) according to the manufacturer's procedure and read on a Multiskan FC plate reader (Thermo Scientific) at 450 nm. All other cytokines were analyzed in 2.5× diluted serum via Mouse Inflammation CBA Kit (BD Bioscience) using a NovoCyte 3000 flow cytometer.

### Evaluation of Cell Activation Using BMDCs

BMDCs were released and plated on 96-well plates at a density of 1.1×10⁶ cells/mL (180 μL) and incubated with PAI (25 μg/mL) or equivalent quantities of unlinked agonists. For Caspase-1 activation, cells were incubated with PAIs for 18 h, and then washed and incubated with caspase-I substrate FAM-YVAD-FMK (FAM-Flica Caspase-I assay kit, Immunochemistry Technologies) according to the manufacturer's protocol to identify the percentage of caspase-I positive cells. For MHC-I antigen presentation, cells were incubated with PAIs for 24 h, then washed and incubated with anti-CD16/32 antibody to block Fc receptors. The cells were subsequently stained with Zombie NIR fixable viability dye (BioLegend) and PE anti-mouse SIINFEKL:H-2Kb antibody. The percentage of live, SIINFEKL:H-2Kb positive cells were then analyzed. Flow cytometry was conducted using a NovoCyte 3000 flow cytometer, and data were analyzed using the NovoExpress software.

### Evaluation of Antigen Processing Using THP-1 Cells

THP-1 cells were plated at a density of 2×10⁶ cells/mL (180 μL) in RPMI+10% HI-FBS and incubated with 25 μg/mL PAI or equivalent amounts of unlinked agonists. The control group received PBS. All cells were simultaneously co-incubated with 10 μg/mL DQ Green BSA (Thermo Fisher) and various agonists for 24 h. Cells were then washed and stained with Hoechst 33,342 and Lysoview 633. Fluorescent images were obtained on a Leica SP5 laser confocal microscope. Each microscopy experiment was performed twice independently. At least 5 different regions were analyzed for each sample. Images were processed with ImageJ software.

### Biodistribution Analysis in Nude Mice

Athymic Nu/J (nude) mice (n=6) were injected subcutaneously with AF647-labelled OVA (20 μg) adjuvanted with PAI (50 μg) or with equivalent quantities of unlinked activators. AF647-labelled OVA (20 μg) was used as a control. Mice were injected subcutaneously with various formulations and fluorescence imaging was performed on an IVIS Spectrum in-vivo imaging system (PerkinElmer). 48 h post-administration of formulations, a cohort of mice from each group were euthanized, and their organs were also imaged with IVIS. Prophylactic vaccination with OVA

Mice (n=5) were vaccinated subcutaneously with OVA (20 μg) adjuvanted with PAI (50 μg) or with equivalent quantities of unlinked 2BXy and TAT-P6-GWWWG. The control group received unadjuvanted OVA in PBS. Mice were given an identical vaccine boost on day 14. On day 24, sera and spleens were collected from mice. Antibody titer was measured by anti-OVA IgG ELISA kit (ADI) and splenic T cell response was measured by intracellular cytokine staining (ICS). For ICS, spleens were processed into a single-cell suspension via mechanical disruption and passed through a 70 μm strainer. The splenocytes were washed with PBS and then treated with ACK lysing buffer (Gibco) for 3 min at room temperature. The single-cell suspension was washed with PBS and resuspended in RPMI. These single cell suspensions were then plated at a density of 5×106 cells/mL in 200 μL and treated with respective peptide epitopes (20 μg/mL). SIINFEKL (Invivogen) was employed as an OVA-specific CD8+ T cell epitope, and ISQAVHAAHAEINEAGR (Invivogen) was employed as an OVA-specific CD4+ T cell epitope. Following 2 h of incubation, 0.2 μL GolgiPlug (BD) was added, and the cells were stimulated for 4 h longer. Following incubation, cells were washed, stained with viability stain, stained for cell surface markers, fixed and permeabilized with Cytofix/Cytoperm kit (BD), and stained for intracellular cytokines. Samples were then analyzed on a NovoCyte 3000 flow-cytometer and analyzed using the NovoExpress software.

Immunotherapy Studies with EG7.OVA Lymphoma

1×10⁵ E G7-OVA cells were injected subcutaneously into the flank of 6-week-old C57Bl/6 mice (n=6 per group) in 100 μL of PBS. Following this OVA (20 μg) adjuvanted with PAI (50 μg) or with equivalent quantities of unlinked 2BXy and TAT-P6-GWWWG were injected subcutaneously on days 5 and 12 post tumor inoculation. The control group received PBS. The tumor size was monitored every alternate day by a singly blinded observer. Tumor volumes were measured using equation V=1/2×L×W×W. Mice were euthanized when the tumors reached 20 mm in any linear dimension.

Immunotherapy Studies with B16.F10 Melanoma

1×10⁵ B16.F10 cells in 100 μL of PBS were injected subcutaneously into the flank of 6-week-old C57Bl/6 J (wild type) or B6.129S6-Nlrp3tm1Bhk/J (NLRP3 knockout) mice (n=7 per group). The tumor size was monitored on alternating days by a singly blinded observer. Tumor volumes were measured using equation V=1/2×L×W×W. When the tumors reached a size of approx. 100 mm3 (day 9), treatment was started. Various NP (75 μg) were formulated with neoantigens (B16-M30, B16-M48, B16-M27; 20 μg each) and injected peritumorally. Simultaneously, treatment groups received intraperitoneal injections of checkpoint blockade antibodies (anti-CTLA4+ anti-PD-L1, 75 μg each). Treatment was repeated on day 15. The control groups received PBS or checkpoint blockade antibodies only. Mice were euthanized when the tumors reached 20 mm in any linear dimension. A parallel study was performed with B16·F10 tumors as described to evaluate antigen-specific T cell production. This study was conducted analogously, and mice were euthanized on day 22. Tumors and spleens were collected. Tumors were smashed, strained through a 70 μm cell strainer, and treated with ACK lysing buffer (Gibco). 2×10⁶ cells from each spleen were plated at a density of 2.5 M cells/mL. Cells were incubated with a cocktail of the three neoantigen peptides (5 μg/mL of each peptide) for 48 h. Cells were then centrifuged at 400×G for 5 min, and the supernatants were collected and analyzed using Mouse TH1/Th2/Th17 cytokine CBA kit (BD Biosciences) following manufacturer's protocol. Granzyme B levels in the supernatant were measured by Granzyme B Mouse ELISA Kit (Thermo Fisher). Finally, tumors were extracted from mice in various treatment groups on day 22. The tissues were sectioned and fixed with 10% neutral buffered formalin solution. Samples were then washed and stored in ethanol following which they were embedded in paraffin and sectioned. Samples were then stained for CD4, CD8 and Foxp3 and imaged using an Axio Observer 7 microscope (Carl Zeiss AG) with an Axiocam 506 color camera (Carl Zeiss AG).

Immunotherapy Studies with CT-26 Carcinoma

1×10⁵ CT-26 cells in 100 μL of PBS were injected subcutaneously into the flank of 6-week-old BALB/c mice (n=10 per group). The tumor size was monitored on alternating days by a singly blinded observer. Tumor volumes were measured using equation V=1/2×L×W×W. When the tumors reached a size of approx. 150-200 mm3 (day 13), treatment was started. PAI (100 μg) was formulated with neoantigens (CT-26-M03, CT-26-M20, CT-26-M90, CT-26-GP70; 20 μg each) and injected peritumorally. Simultaneously, treatment groups received intraperitoneal injections of checkpoint blockade antibodies (anti-CTLA4+ anti-PD-L1, 75 μg each). The control groups received either PBS or antigen cocktail or checkpoint blockade antibodies only. Treatment was repeated on day 18 and day 23. Mice were euthanized when the tumors reached 20 mm in any linear dimensions. The tumors were monitored until day 60, when all surviving animals were tumor free. Remaining surviving mice in PAI groups were reinjected with 1×10⁵ CT-26 cells and monitored for tumor growth until day 90.

Toxicity Studies with CT-26 Carcinoma

Mice were injected with CT-26 cells and vaccinated on day 13 as described above. Plasma was collected by submandibular bleed 2 h post-injection of formulations on day 13 for cytokine analysis. 2 d post-injection, blood was collected from animals by submandibular bleed in EDTA coated Eppendorf tubes (Fisher Scientific). Samples were immediately analyzed for complete blood count (CBC) using a Hemavet 950 instrument (Drew Scientific). The instrument was fitted with a reagent pack obtained from the manufacturer. Prior to analysis a blank run and a quality control run (using manufacturer provided control sample) were performed to ensure optimal performance by the instrument. 20 μL of blood were injected for each analysis using a sample cycle of approximately 2 min.

### Mass Spectrometry

Electrospray ionization mass spectrometry (ESI-MS) was conducted using a Waters LCT Premier™ XE system. Matrix-assisted laser desorption/ionization mass spectrometry (MALDI-MS) was conducted using a Bruker Ultraflextreme MALDI-TOF-TOF system using Super DHB (Supelco) as a solid support matrix.

### Nuclear Magnetic Resonance Spectroscopy

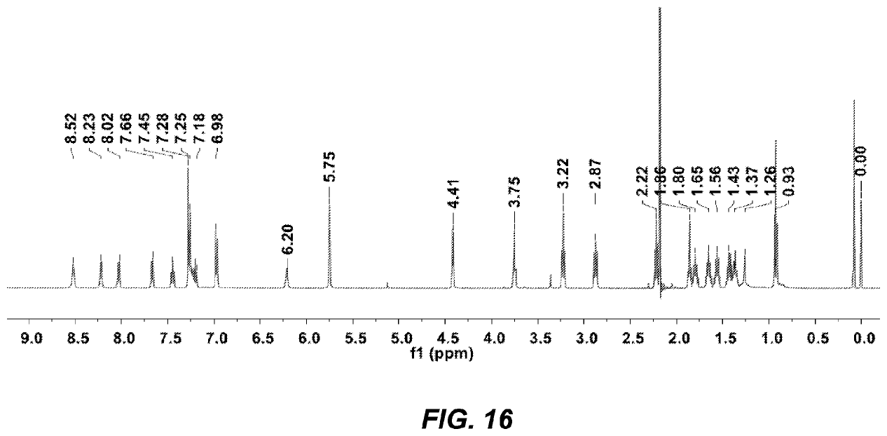

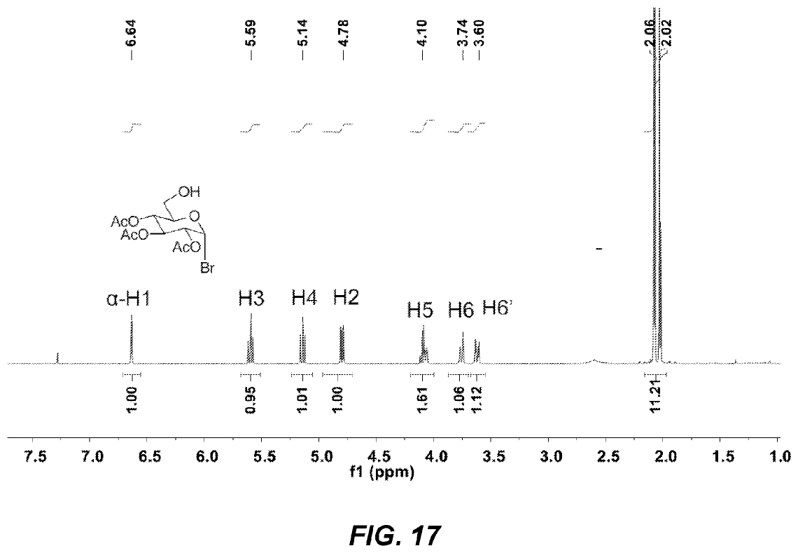

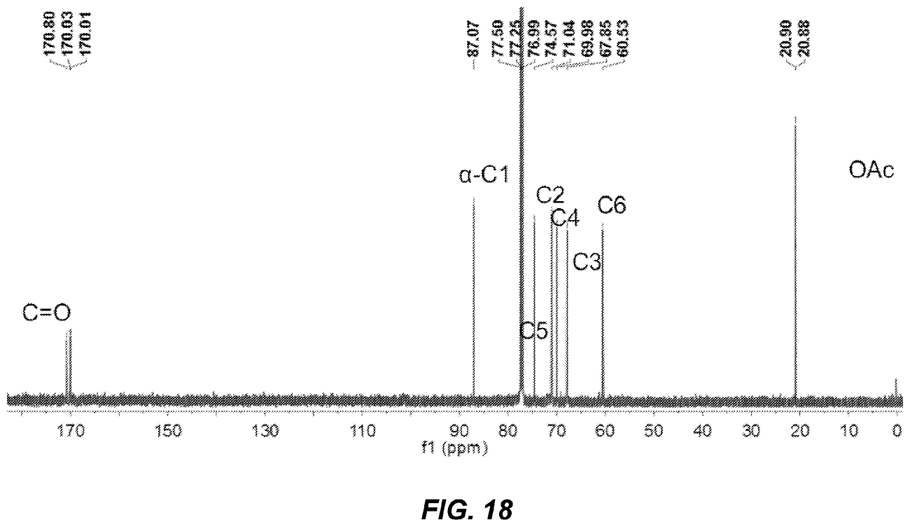

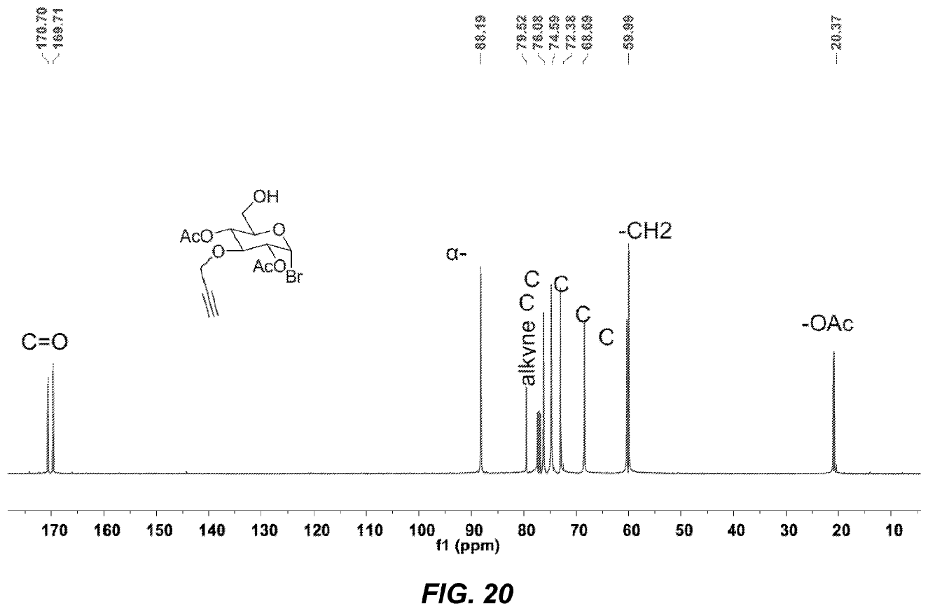

Proton nuclear magnetic resonance (¹H NMR) spectra were recorded at 300 MHz on a Varian Mercury 300 or 500 MHz on a Varian Inova 500 spectrometer or a Bruker 500 spectrometer, with tetramethylsilane (TMS) proton signal as the standard (FIGS. 16, 17, 19, and 21). ¹³C NMR spectra were recorded at 126 MHz on a Varian Inova 500 spectrometer, with tetramethylsilane (TMS) carbon signal as the standard (FIGS. 18 and 20)

### Gel Permeation Chromatography

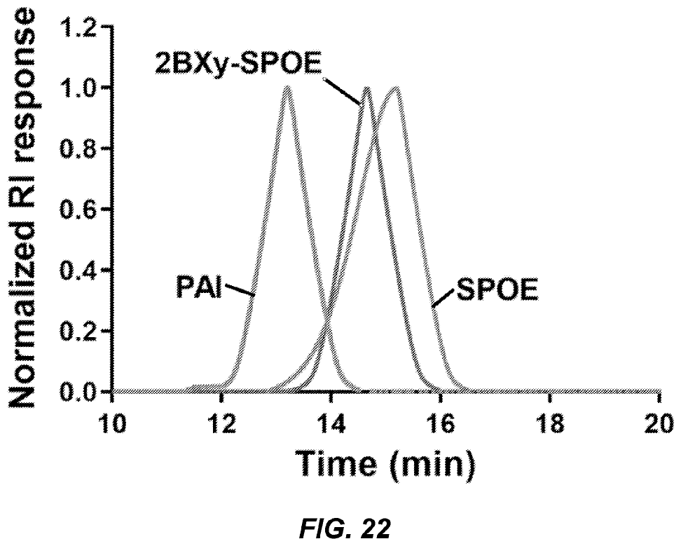

Gel permeation chromatography (GPC) analyses (FIG. 22) were conducted using a Viscotek GPC system equipped with a VE 3580 RI detector, VE 112 solvent delivery system, and a column system comprised of one PAS102 and one PAS103 column (Polyanalytik Inc.). The system was equilibrated at 35° C. in DMF, which served as the polymer solvent and eluent with a flow rate of 1.0 mL min-1. Polymer solutions were prepared at a known concentration (ca. 6 mg/mL) and an injection volume of 100 μL was used. Data collection and analyses were performed by OmniSEC software system from Malvern Inc. The GPC system was calibrated using polystyrene standards having molecular weights of 2.5 5.0, 9.0, 17.0 and 50.0 kDa and PDI of 1.05-1.07 (Supelco Analytical, Bellefonte, PA, USA).

### Transmission Electron Microscope

Transmission Electron Microscopy (TEM) measurements were performed using a FEI Tecnai F30 300 kV FEG(s) TEM microscope. Carbon-coated copper grids were treated with oxygen plasma before deposition of the samples. The samples were deposited on the carbon grids for 1 min, and excess samples were wicked away. The samples were allowed to dry under ambient conditions. Samples were stained with uranyl acetate.

### Dynamic Light Scattering

Dynamic light scattering (DLS) measurements were performed by a Wyatt Mobius DLS instrument. Measurements were performed at 25° C. using a laser wavelength of 532 nm. Scattered light was collected at a fixed angle of 163.5°.

### High Performance Liquid Chromatography

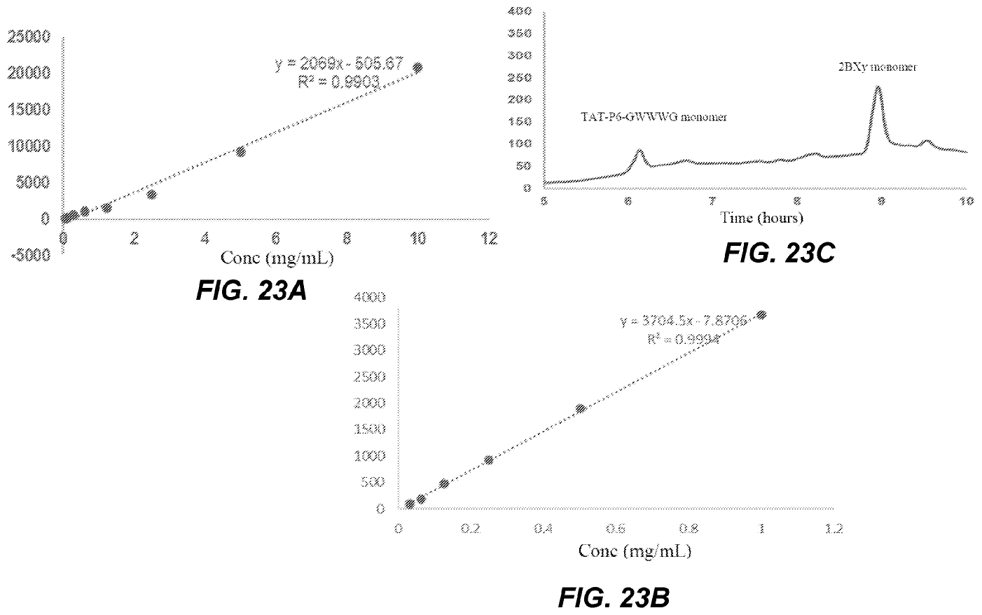

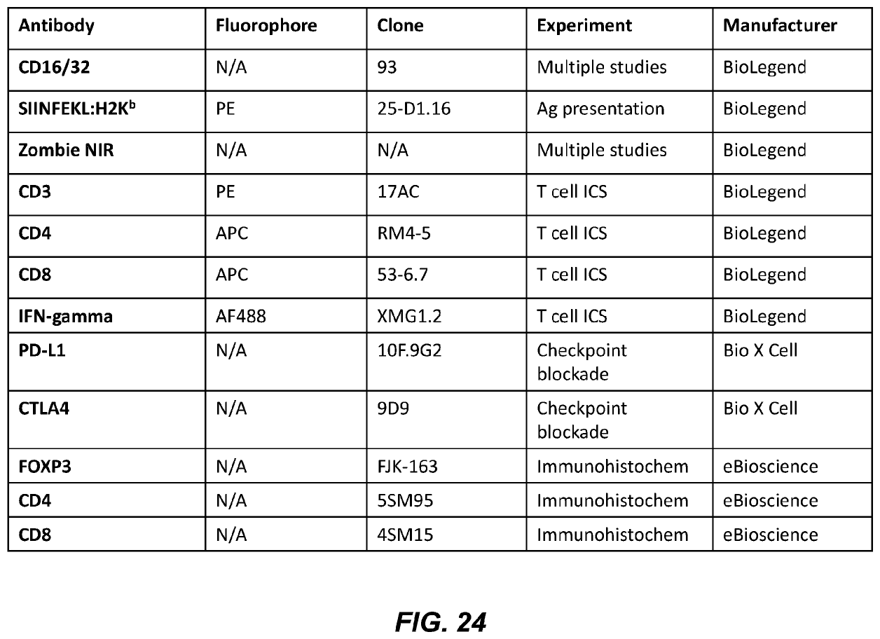

High performance liquid chromatography (HPLC) was employed to evaluate the composition of PAIs using an Agilent Infinity 1260 analytical HPLC equipped with a Phonomenex Luna C18 column (5 μm, 100 A, 150×4.6 mm) and a UV-VIS detector. First, Free TAT-P6-GWWWG and 2BXy were injected to determine the elution time and molar absorption coefficient at 280 nm of each component. Then, PAIs were degraded by incubating in 0.1% TFA in 1:1 DMSO:water for 4 h. Following TFA treatment, the degraded PAI solution was injected to the HPLC, and the area under the curve of each component peak was referenced to the standard curves to quantify the presence of TAT-P6-GWWWG and 2BXy in the polymer scaffold (FIGS. 23A-23C).

### Solid Phase Peptide Synthesis

Fmoc-solid phase peptide synthesis of TAT-P6-GWWWG and neoantigen peptides was performed using a Liberty Blue peptide synthesizer (CEM) using Rink amide resin (100 200 mesh) as the solid support. For the synthesis of TAT-P6-GWWWG, the N-terminus was capped with azidohexanoic acid to allow conjugation to the alkyne-containing SPOE scaffold. Double coupling and extended coupling times were used to couple the arginines and the hexaethylene glycol linker. Peptides were deprotected using a mixture of 85% TFA mixed with 5% water, 5% anisole, and 5% thioanisole. Following deprotection, thecrude peptide was precipitated in cold diethyl ether. The crude peptide was then purified using a Phonomenex Luna C18 column (5 μm, 100 A, 150×21.2 mm) on a Gilson preparative HPLC using a gradient of acetonitrile (containing 0.1% TFA) in water (containing 0.1% TFA) over a period of 15 min at a flow rate of 21.2 mL/min. The purified peptides were lyophilized and characterized using MALDI-MS.

### Synthesis of 2BXy-Azide

Compound 2a (100 mg, 0.28 mmol) and azidohexanoic acid (48.41 mg, 0.31 mmol) were added in anhydrous DMF following which DIPEA (73.2 μL, 0.42 mmol and HATU (159.70 mg, 0.42 mmol) were added to the reaction mixture under argon. The reaction mixture was stirred for 12 h at room temperature following which the solvent was removed under vacuo and the product was purified by silica gel column chromatography using a gradient of 0-5% methanol in dichromethane, giving compound 2 as a yellow powder (104.8 mg, 0.21 mmol, 75% yield). 1H NMR (500 MHz, CDCl₃) δ 8.52 (s, 1H), 8.22 (d, J=8.3 Hz, 1H), 8.03 (d, J=8.3 Hz, 1H), 7.67 (d, J=8.3 Hz, 1H), 7.44 (t, J=8.3 Hz, 1H), 7.25 (d, J=8.1 Hz, 2H), 7.20 (m, 1H), 6.97 (d, J=8.1 Hz, 2H), 6.21 (s, 1H), 5.75 (s, 2H), 4.41 (d, 2H), 3.75 (m, 2H), 3.22 (t, 2H), 2.87 (t, 2H), 2.22 (m, 2H), 1.86 (s, 2H), 1.80 (m, 2H), 1.65 (m, 2H), 1.56 (m, 2H), 1.42 (m, 2H), 1.38-1.33 (m, 2H), 1.26 (s, 1H) 0.93 (t, 3H). ESI-MS: m/z calculated for C₂₈H₃₄₊₁N₈O: 499.64, observed: 499.65.

### Synthesis of 2,3,4-tri-O-acetyl-α-D-glucopyranosyl bromide (SPOE Monomer I)

In a 50 mL disposable polypropylene tube, 2,3,4-tri-O-acetyl-6-O-tert-butyldiphenylsilyl-α-Dglucopyranosyl bromide (1.0 g, 1.65 mmol) was added to anhydrous tetrahydrofuran (7 mL). HFPy (0.82 g, 70% HF in pyridine, 0.57 g HF, 29 mmol HF) was subsequently added. The reaction was stirred under nitrogen at 4° C. for 12 h. The solvent and excess HF-Py was removed by sparging N2 gas at 4° C. and the residue was purified by silica gel column chromatography (hexanes/ethyl acetate=6/4, Rf=0.3) to afford the product as a white powder (0.38 g, 62%). 1H NMR (300 MHz, CDCl₃) δ 6.64 (d, J_{1,2}=4.0 Hz, 1H, α-H1), 5.63 (dd, J_{2,3}=10 Hz, J_{3,4}=10 Hz, 1H, H-3), 5.14 (dd, J_{3,4}=10 Hz, J_{4,5}=10 Hz, 1H, H-4), 4.80 (dd, J_{2,3}=10 Hz, J_{1,2}=4.0 Hz, 1H, H-2), 4.09-4.06 (m, 1H, H-5), 3.78 (ddd, J_{6,6′}=13.2 Hz, J_{6,OH}=8.2 Hz, J_{5,6}=2.2 Hz, 1H, H-6), 3.63 (ddd, J_{6,6′}=13.2 Hz, J_{6′,OH}=6.2 Hz, J_{5,6}=3.5 Hz, 1H, H-6), 2.29 (dd, J_{6,OH}=8.2 Hz, J_{6′,OH}=6.2 Hz, 1H, —OH), 2.10 (s, 3H), 2.09 (s, 3H), 2.05 (s, 3H). ¹³C NMR (126 MHz, CDCl₃) δ 170.80 (C═O), 170.03 (C═O), 170.01 (C═O), 87.07 (α-C1), 74.57 (C-5), 71.04 (C-2), 69.98 (C-3), 67.85 (C-4), 60.53 (C-6), 20.90 (—OAc), 20.88 (—OAc). ESI-MS calc for C₁₂H₁₇BrNaO₈ [M+Na]⁺=391.00, 393.00, found: 391.12, 393.13.

### Synthesis of 3-propargyl-2,4-di-O-acetyl-α-D-glucopyranosyl bromide (SPOE monomer II)

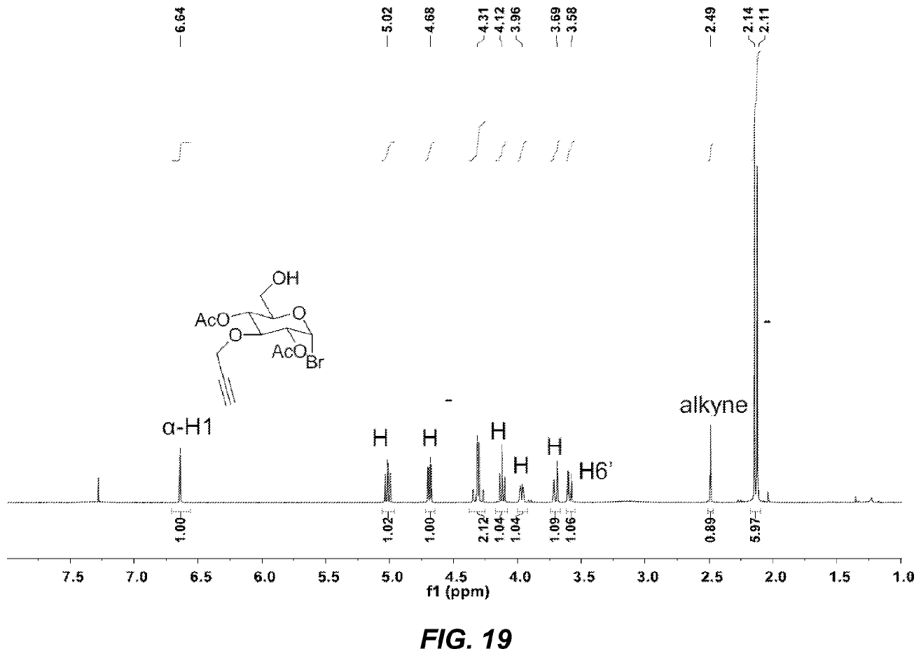

In a 50 mL disposable polypropelene tube, 3-propargyl-2,4-di-O-acetyl-6-O-tert-butyldiphenylsilyl-α-D-glucopyranosyl bromide (1.0 g, 1.7 mmol) was added to anhydrous tetrahydrofuran (7.0 mL). HF-Py (0.82 g, 70% HF in pyridine, 0.57 g HF, 29 mmol HF) was subsequently added. The reaction was stirred under nitrogen at 4° C. for 12 h. The solvent and excess HF-Py was removed by sparging N2 gas at 4° C. and the residue was purified by silica gel column chromatography (hexanes/ethyl acetate=6/4, Rf=0.3) to afford the product as a white powder (0.40 g, 66%). FIG. 19 ¹H NMR (500 MHZ, CDCl₃) δ 6.64 (d, J=3.9 Hz, 1H, α-H1), 5.02 (m, 1H, H-4), 4.68 (dd, J=9.6, 3.9 Hz, 1H, H-2), 4.31 (m, 2H, —CH2), 4.12 (dd, J_{2,3}=10 Hz, J_{3,4}=10 Hz, 1H, H-3), 3.96 (m, 1H, H-5), 3.69 (ddd, J_{6,6′}=13.2 Hz, J_{6,OH}=8.2 Hz, J_{5,6}=2.2 Hz, 1H, H-6), 3.58 (ddd, J_{6,6′}=13.2 Hz, J_{6′,OH}=6.2 Hz, J_{5,6}=3.5 Hz, 1H, H-6′), 2.49 (t, J=2.4 Hz, 1H, HC═C), 2.13 (d, J=10.0 Hz, 6H), 2.14 (s, 3H), 2.11 (s, 3H), FIG. 20 ¹³C NMR (126 MHz, CDCl₃) δ 170.66 (C═O), 169.27 (C═O), 88.47 (α-C1), 79.54 (acetylene —CH), 76.33 (C-3), 74.77 (C-5), 72.73 (C 2), 68.69 (C-4), 61.08 (O—CH2), 60.31 (C-6), 20.86 (—OAc). ESI-MS calc for C₁₃H₁₇BrNaO₇ [M+Na]⁺=388.17, found: 388.06.

### Synthesis of Sugar Poly(Orthoester) (SPOE)

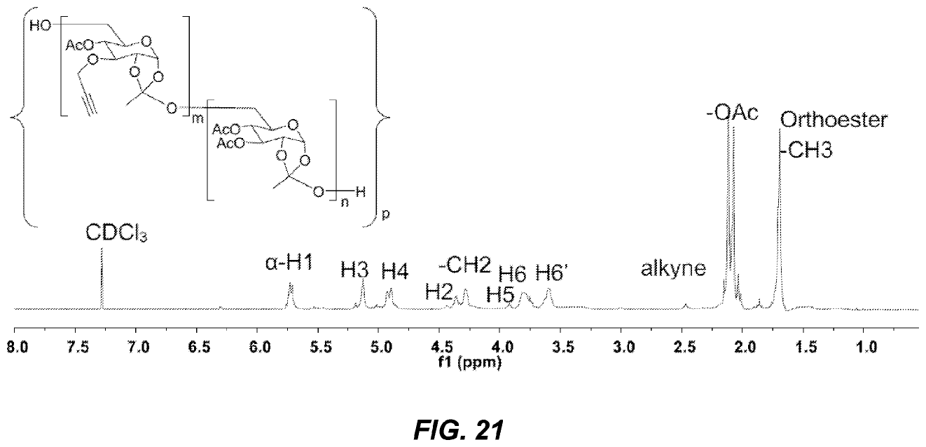

To a Schlenk flask was added monomer I (0.20 g, 0.54 mmol), monomer II, (0.05 g, 0.11 mmol), anhydrous CH₂Cl₂ (5 mL), tetrabutylammonium iodide (TBAI) (0.199 g, 0.54 mmol) and N,Ndiisopropylethylamine (DIPEA, 0.21 g, 1.63 mmol). The reaction mixture was refluxed under argon atmosphere for 20 h. The solvent was removed by reduced pressure. The polymer was precipitated three times using a mixture of water/methanol (9/1, v/v) at 4° C. to afford 1 as a white powder (0.18 g, 70%). MnGPC=6.3 kDa, PDI=1.3. FIG. 21 ¹H NMR (500 MHz, CDCl3) δ=5.71 (d, J=5.0, 1H, α-H1), 5.13 (m, 1H, H-3), 4.92-4.90 (t, J=9.0, 1H, H-4), 4.42 (s, —CH2), 4.27 (m, 1H, H-2), 3.80-3.78 (m, 1H, H-5), 3.62-3.55 (m, 2H, H-6,6′), 2.44 (t, acetylene proton), 2.11 (m, 3H), 2.07 (s, 3H), 1.69 (s, 3H). ¹³C NMR (126 MHZ, CDCl3) 169.99 (C═O), 169.51 (C═O), 121.34 (orthoester C), 97.30 (C1), 80.06 HC≡C), 72.81 (C-2), 70.09 (C-3), 68.35 (C-4), 67.81 (C-5), 63.25 (C-6), 21.12 (—OAc), 20.68 (—OAc), 20.59 (—CH3).

### Analysis of Encapsulation Efficacy of Neoantigen Peptides

PAI (750 μg) was incubated to 200 μg of each peptide in a total volume of 1 mL DI water. The solution was then centrifuged at 5000×g to precipitate out the particles. The supernatant was then injected to an Agilent Infinity 1260 analytical HPLC equipped with a Phonomenex Luna C18 column (5 μm, 100 A, 150×4.6 mm) and a UV-VIS detector set to 280 nm, and the free peptide in solution was quantified by HPLC analysis relative to dissolved peptide standards.

### Example 2—Generation and Characterization of Inflammasome Activating Nano-Vaccines

2BXy activates endosomal TLR 7/8 receptor in APCs and has been previously reported to induce robust anti-tumor cellular immunity, whereas previous in-vitro & in-vivo studies with the endo-osomolytic TAT-GWWWG peptide demonstrated robust NLRP3 inflammasome activation. Disclosed is a design of a multicomponent vaccine adjuvant platform composed of small molecule TLR activator 2BXy and peptide based NLRP3 inflammasome activator (TAT-GWWWG) organized on a polymeric scaffold.

### Synthesis of a Series of Polymeric Activators with Varying Functional Group Ratios

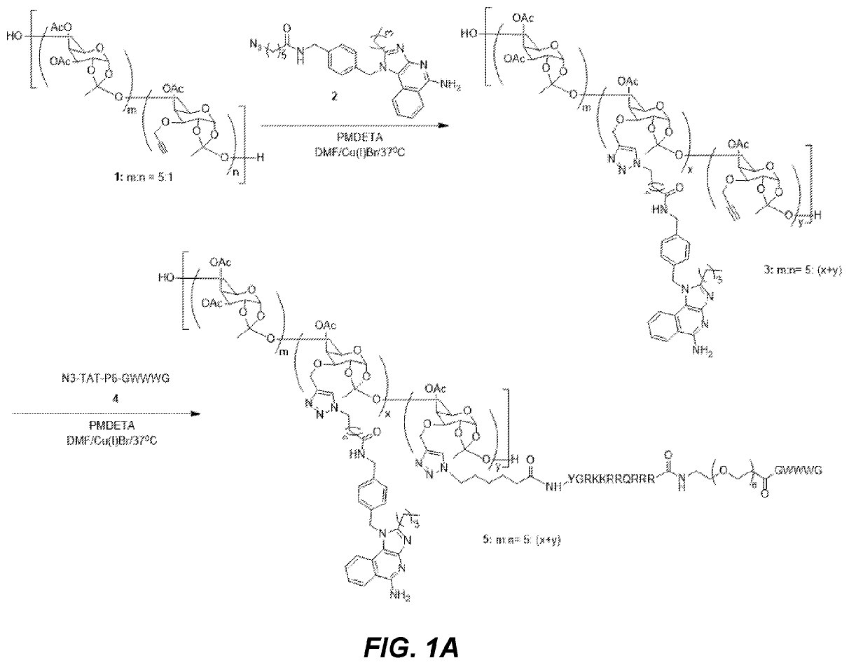

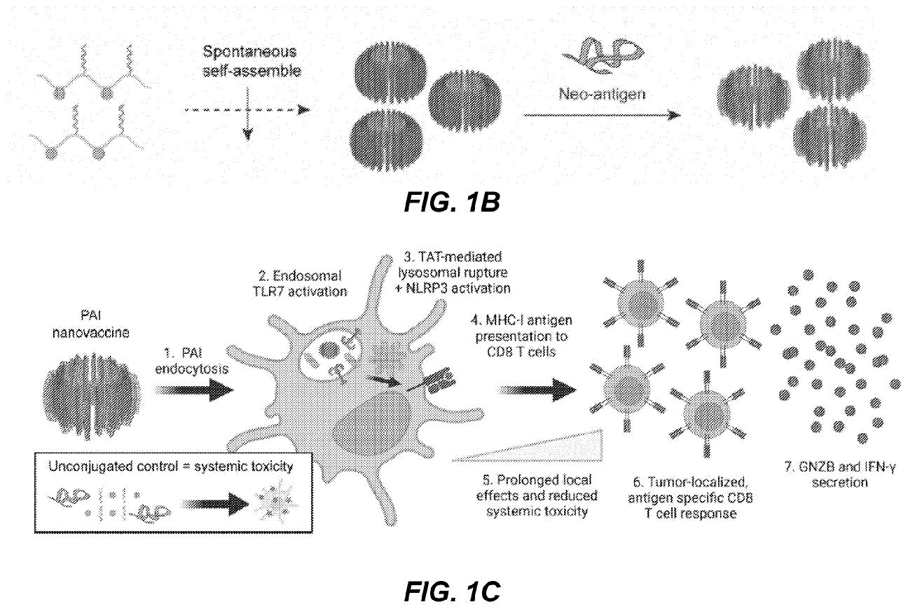

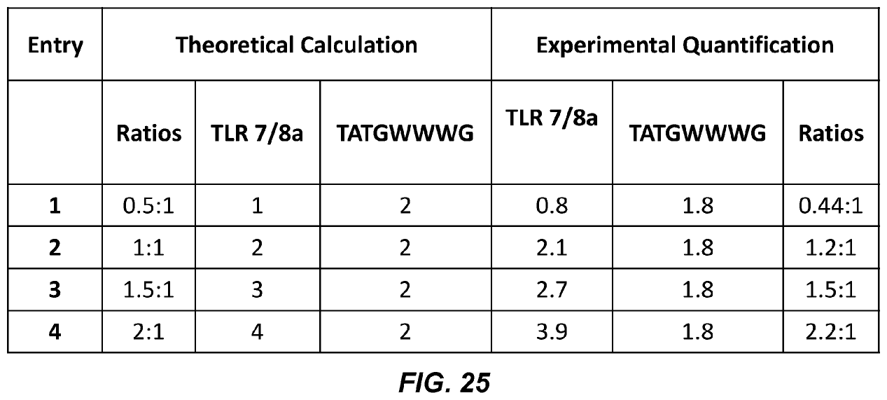

With the goal of generating inflammasome activating immunostimulant platform variable ratios of azido-functionalized 2BXy and azido-functionalized TAT-GWWWG peptide were grafted to a non-immunogenic carbohydrate scaffold (SPOE) via sequential Cu(I) catalyzed Huisgen cycloaddition chemistry (FIG. 1A). Such a design afforded a series of amphiphilic polymers with varying ratios of 2BXy and TAT-GWWWG (FIG. 25). Owing to the polymeric nature of these immune activators, such molecules are referred to herein as Polymeric Activators of the Inflammasome (PAIs). The ratio of 2BXy and TAT-GWWWG peptide in the synthesized PAIs were quantified employing HPLC following degradation of pH-sensitive ortho-ester linkages via TFA.

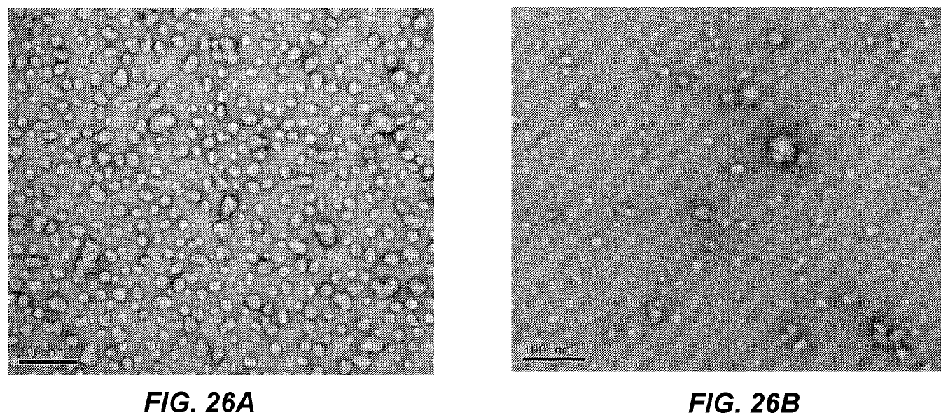

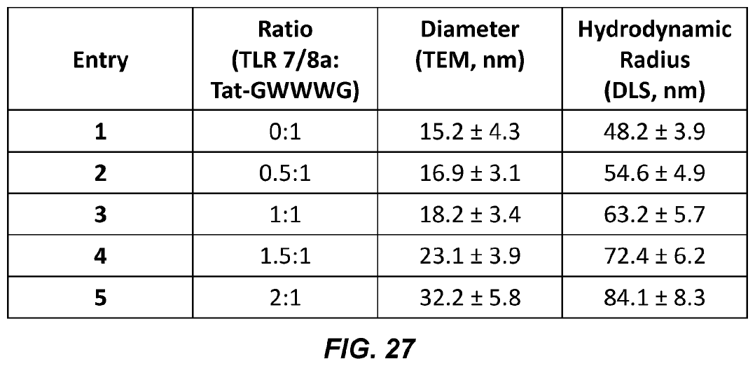

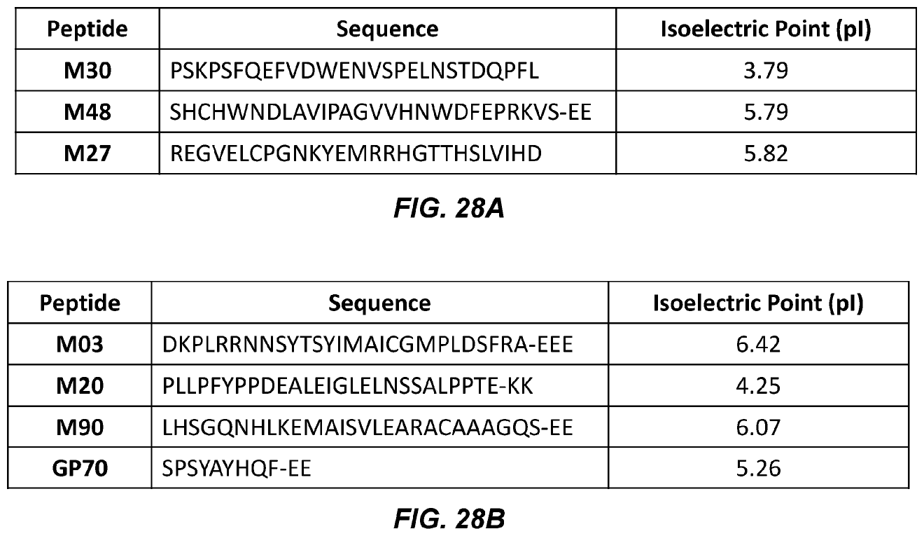

With these PAIs in hand, the synthesis of nanoparticles (NPs) was performed through self-assembly (FIG. 1B). The amphiphilic nature of the polymer-conjugates led to generation of well-defined nano micelles. Depending on the ratios of 2BXy and peptide TAT-GWWWG, NPs of varying TEM sizes were obtained (FIGS. 26A-26B and FIG. 27). Notably, all the particles remained quite stable, and no significant structural or functional changes were observed when stored at 4° C. for a period of at least 8 weeks.

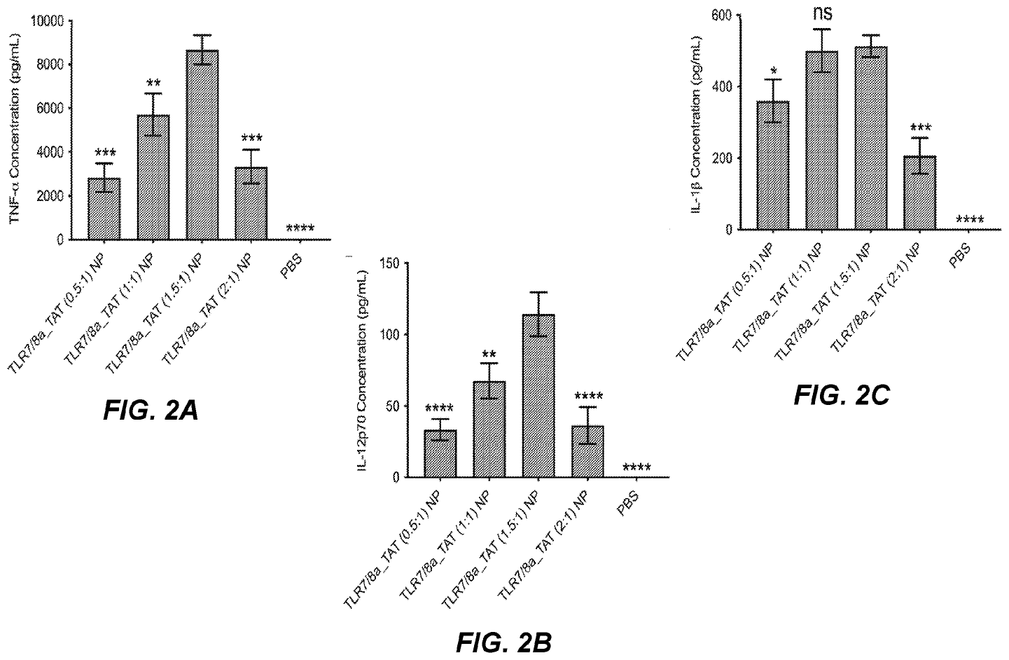

With the PAIs in hand, their ability to elicit an immune response was evaluated. For this study, PAI nanoparticles were incubated with Bone Marrow Derived Dendritic Cells (BMDCs) and the cytokine secretion were analyzed in supernatants (FIGS. 2A-2C). In this case, apart from analysis of IL-1b (IL-1B) to measure for inflammasome activation, a series of proinflammatory cytokines were also investigated. This study indicated that PAI with a ratio of 1.5:1 of 2Bxy and TAT-GWWWG generated the highest amount of IL-1b along with other cytokines. Notably, this ratio elicited highest secretion of TNF-a (TNF-α) and IL-12 which play a key role in antigen presenting cell (APC) activation and cancer immunosurveillance. Therefore, this PAI (1.5:1) ratio was chosen for further biological studies.

### PAI Induces NLRP3 Inflammasome Activation

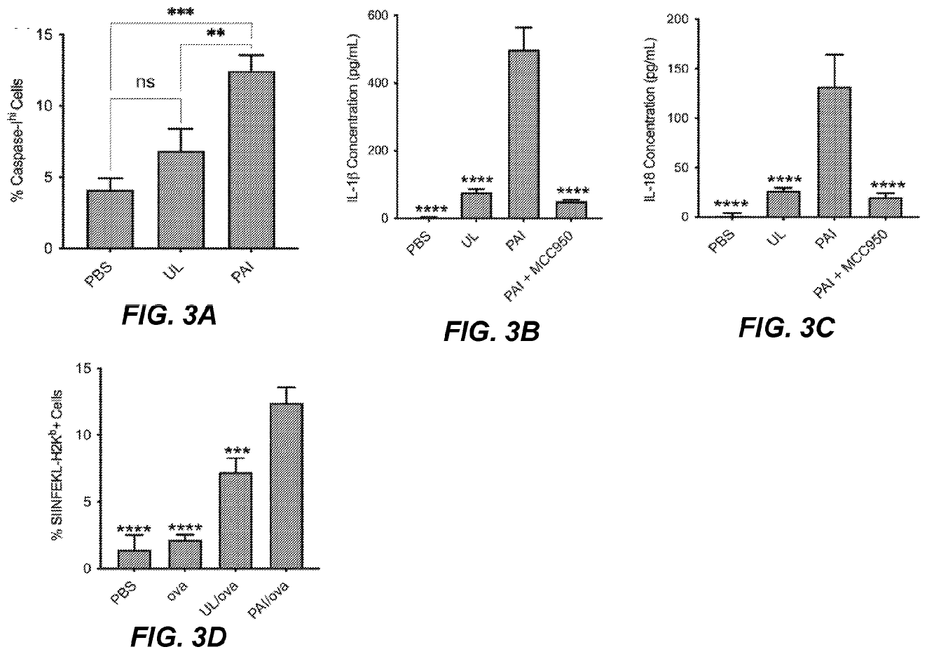

With this encouraging initial data, detailed evaluation of inflammasome activation was performed using the chosen PAI (1.5:1) (hereafter referred as PAI) in comparison with equivalent amount of unliked agonist mixtures and PBS. Caspase-I enzyme activity was evaluated along with secretion of IL-1b and IL-18 cytokines in BMDCs to analyze for inflammasome activation. It was observed that, compared to unlinked agonist treated group, PAI induced significantly higher caspase-I enzyme activity (FIG. 3A) along with 10-fold higher IL-1b and 5-fold higher IL-18 secretion (FIGS. 3B and 3C).

Additionally, to explore whether the responses are specific to the NLRP3 activation, BMDCs were co-incubated with NLRP3-specific inhibitor MCC-950 along with PAI. Co-incubation with MCC-950 resulted in a significant reduction of IL-1b and IL-18 secretion (FIGS. 3B and 3C) indicating the response is specific to NLRP3 activation. Overall, these studies indicated robust NLRP3 inflammasome activation by PAI.

### PAI Induced Enhanced Lysosomal Escape and Antigen Cross Presentation

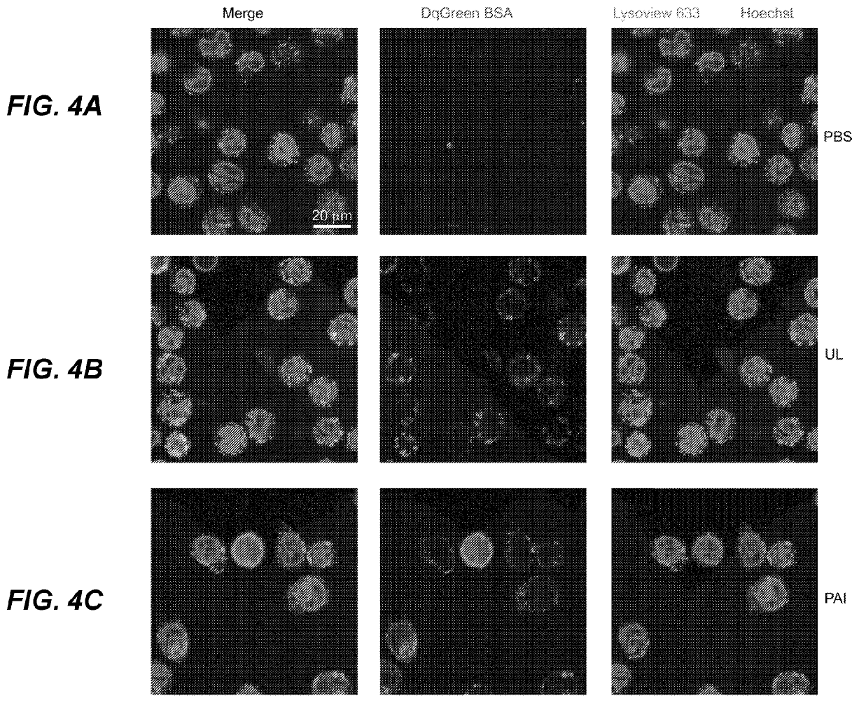

The results from the inflammasome activation analysis showed great promise. Due to the known lysosomolytic property of the TAT-P6-GWWWG peptides, the inventors were also interested to evaluate the lysosomal rupture activity of PAI as lysosomal rupture and cytosolic delivery can enhance antigen presentation in APCs leading to potent downstream immune response. The inventors thereby incubated PAI or equivalent amount of unlinked agonist mixtures with a fluorogenic protein antigen (DQ Green BSA) on THP-1 cells to determine the efficacy of lysosomal proteolysis. Confocal microscopy analysis indicated that PAI induced significantly higher cytosolic diffusion of DQ-Green BSA (FIG. 4C) whereas in PBS and unlinked agonist treated cells DQ green BSA were mostly confined in distinct punctate lysosomes (FIGS. 4A and 4B). These results thereby indicated more extensive lysosomolysis by PAI formulations.

With this promising observation, the next goal was to evaluate whether increased cytosolic delivery of protein antigens by PAI could enhance antigen cross-presentation. In this study, PAI formulation was incubated with BMDCs along with Ovalbumin (OVA) antigen, following which cells were then stained for antigen presentation (SIINFEKL-H-2Kb complexes) on the surface of DCs (FIG. 3D). Parallel studies were performed with unlinked agonist formulation in combination with OVA or with OVA in PBS. Flow cytometry analysis demonstrated that PAI-OVA treated cells exhibited a significantly higher level of SIINFEKL-H-2Kb complex compared to other treatment groups (FIG. 3D). This indicated that the PAI formulation can potentially induce higher downstream antigen-specific immune responses. These results thereby validated the hypothesis that co-activation of TLR and NLRP3 by PAI can induce potent immune activation and antigen processing by APCs.

### PAI Induces Antigen Localization and Delivery

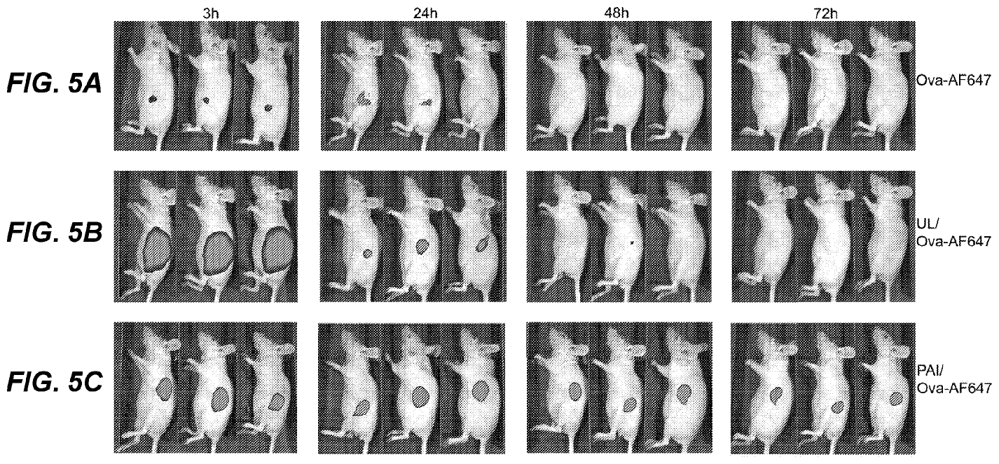

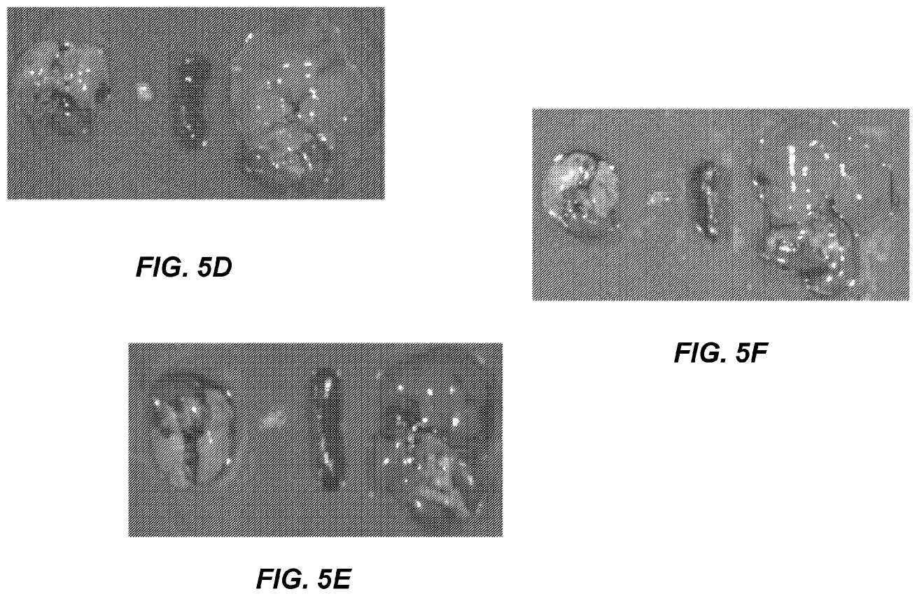

With the promising in-vitro results, the inventors were very interested to evaluate the in-vivo biodistribution of PAI when formulated with antigens. Thereby, PAI was admixed with AF-647 labelled OVA antigen to promote antigen adsorption. DLS and TEM analysis indicated that the PAI particles were stable in formulation. Identical formulations were also developed with AF-647 OVA antigen in PBS or with AF-647 OVA formulated with equivalent quantities of unlinked activators. The formulations were then injected subcutaneously in the flank of athymic nude mice (n=6) and biodistribution of antigen-formulations were monitored via IVIS imaging at regular intervals (FIGS. 5A-5C). It was observed that, compared with other treatment groups, PAI formulations induced significant antigen localization at the injection site. Notably, while free antigen in PBS and unlinked agonist-antigen formulation treated animals had undetectable levels of antigen 48 hr post-injection, PAI formulations demonstrated significant antigen localization even after 72 hr post-injection (FIG. 5C). To further explore the effect of PAI formulations, organs were isolated from a cohort of treated animals 48 h post-injection and analyzed for bio-distribution of AF-647 (FIGS. 5D-5F). Notably, the PAI group (FIG. 5F) demonstrated significant localization of PAI-OVA formulations in the draining inguinal lymph node, while detectable levels of antigens were not observed in other organs. Consistent with previous observation, antigens were also not detectable in organs isolated from unlinked agonist-OVA (FIG. 5E) or PBS-OVA (FIG. 5D) treated groups 48 h post-injection. These studies thereby indicated PAI-antigen formulations induced improved localization at the site of injection and draining lymph nodes compared to unlinked agonist formulation.

### PAI Enhances Vaccine Immunogenicity and Anti-Tumor Efficacy

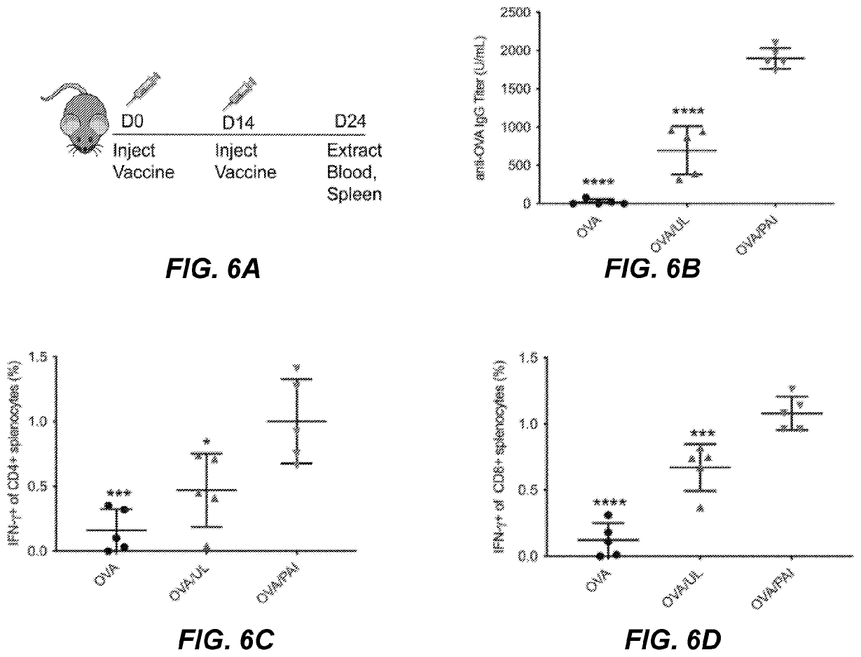

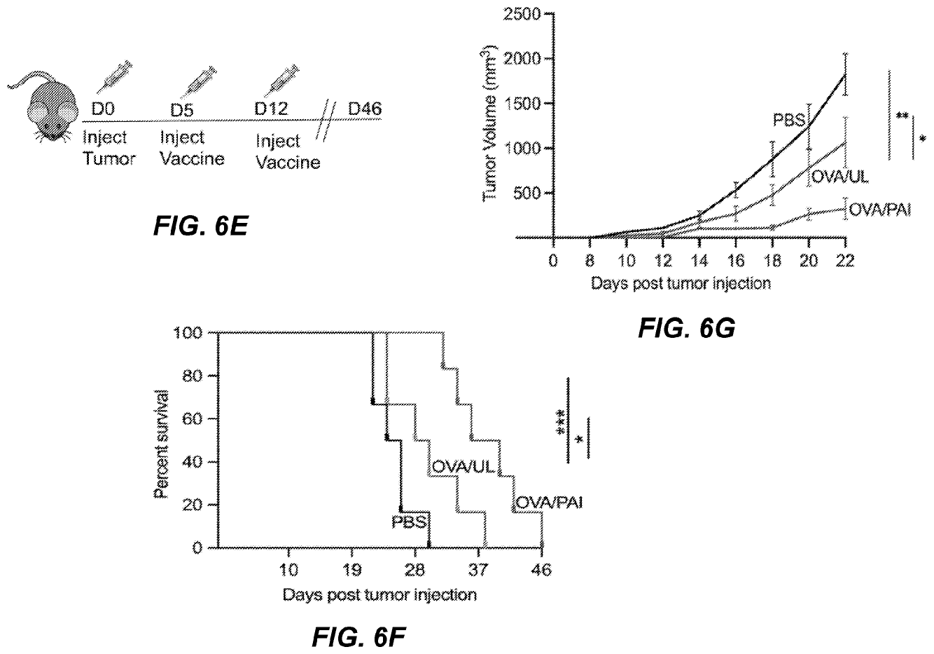

With the promising in-vitro studies and biodistribution analysis, the inventors were motivated to evaluate the efficacy of PAI in enhancing immunogenicity of antitumor vaccines. Thereby, initially in-vivo studies were performed to understand vaccine immunogenicity of OVA-PAI formulation in comparison with unadjuvanted OVA in PBS or OVA formulated with equivalent quantities of unlinked activators. Mice (n=5) were injected subcutaneously with each formulation followed by a subsequent, identical boost on day 14. On day 24 blood sera were collected to analyze for antibody titers and splenocytes were harvested to analyze antigen-specific T-cell responses. It was observed that PAI significantly enhanced antibody titers compared to unlinked agonist (173±22%) or unadjuvanted (9362±312%) formulations (FIG. 6B). Additionally, analysis of antigen-specific splenocytes revealed that PAI formulation enhanced IFN-g (IFN-γ) secreting CD4+ T-cell response by (113±41%) and IFN-g secreting CD8+ T-cell response by (61±14%) compared to unlinked formulation (FIGS. 6C and 6D). These results thereby indicated that, compared with unadjuvanted antigen or unlinked agonist formulations, PAI significantly enhanced antigen-specific immune responses to vaccination.

With the promising analysis of immunogenicity PAI in the OVA vaccination studies, the inventors next proceeded to investigate the application of PAI in enhancing antigen-specific immune responses in cancer vaccines. First, studies were performed in an EG7.ova lymphoma model in which mice (n=6) were implanted with EL4 cells that stably express OVA protein (FIGS. 6E-6G). In this study tumors cells were injected on the right flank of mice on day 0 following which vaccines were administered subcutaneously on day 5 and day 12 with OVA loaded PAI or OVA with unlinked agonist formulations. The control group received PBS. Mice were monitored for tumor growth and sacrificed when tumor size reached 20 mm in any linear dimension. It was observed that administration of PAI formulations significantly reduced tumor burden and prolonged survival compared to other formations (FIGS. 6F and 6G). Notably, animals in PAI formulation treated group had a median survival of 38 days which was significantly higher compared to median survival of 29 days for unlinked agonist formulation and 25 days for PBS treated animals. The enhanced efficacy of PAI formulation is thereby consistent with the previous observation of enhanced antigen-specific immune responses of PAI formulations in OVA vaccination studies.

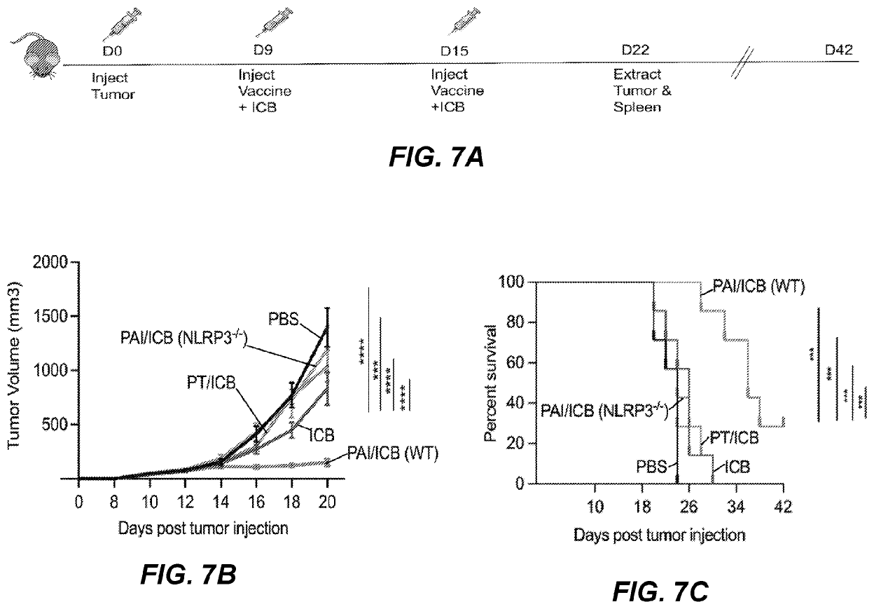

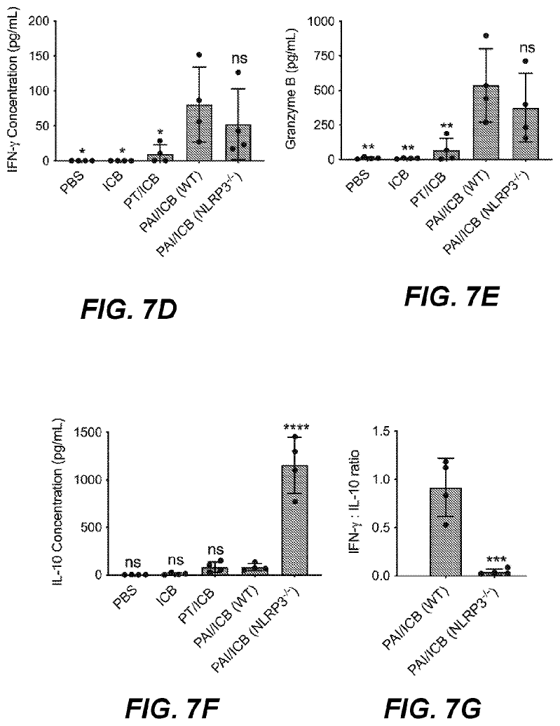

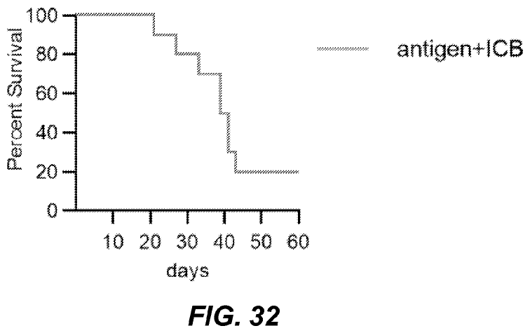

With these exciting findings, the inventors were very interested to evaluate the efficacy of PAI formulations to induce anti-tumor immunity against tumor-specific neo-antigens in a B16.F10 murine melanoma model. This model has been widely investigated as a poorly immunogenic and highly aggressive murine tumor model and has been observed to be unresponsive to various immunotherapeutic modalities including clinically approved checkpoint blockade therapies. Thereby, the effect of PAI formulated vaccines in reducing tumor burden in combination with immune checkpoint blockade therapy (ICB:anti-CTLA4+ anti-PDL1) was investigated. For this study, PAI was formulated with multiple B16.F10 neoantigen peptides. These peptides were modified with glutamate linkers to promote adsorption with PAI in formulation. Parallel studies were also performed with NLRP3 deficient mice to investigate the role of inflammasome activation with PAI formulations. Moreover, to differentiate between the effects of adjuvanticity and carrier properties of the PAI scaffold, an analogous nano-formulation (PT) was developed by grafting equivalent quantities of TAT-GWWWG peptide to the SPOE scaffold. For comparison, studies were also performed with ICB antibodies and unlinked agonist formulation in combination with ICB. Mice (n=7) were injected with tumor cells on the right flank on day 0 and vaccine formulations were injected peritumorally on day 9, when tumors started appearing, followed by a boost injection on day 15. Along with the various vaccine formulations ICB antibodies were administered intraperitoneally. The study was concluded at day 42 when all the treated animals were either observed to be tumor free or were sacrificed due to large tumors (20 mm in any linear dimension). It was observed that the PAI vaccines in combination with ICB significantly reduced tumor burden and prolonged survival resulting in complete tumor remission in 30% of treated animals on day 42 (FIGS. 7B and 7C and FIG. 32). Notably, treatment with the PAI vaccine improved median survival to 36 days compared to 24 days for PBS treated animals and 26 days for animals treated with ICB only. In comparison, neither the PAI-ICB combination therapy in NLRP3-deficient mice, nor the PT-ICB combination therapy improved survival compared to treatment with ICB alone. Moreover, with this treatment regime, unlinked activator formulations induced severe toxicity resulting in severe weight loss (>20%) and death and hence were not included in further studies.

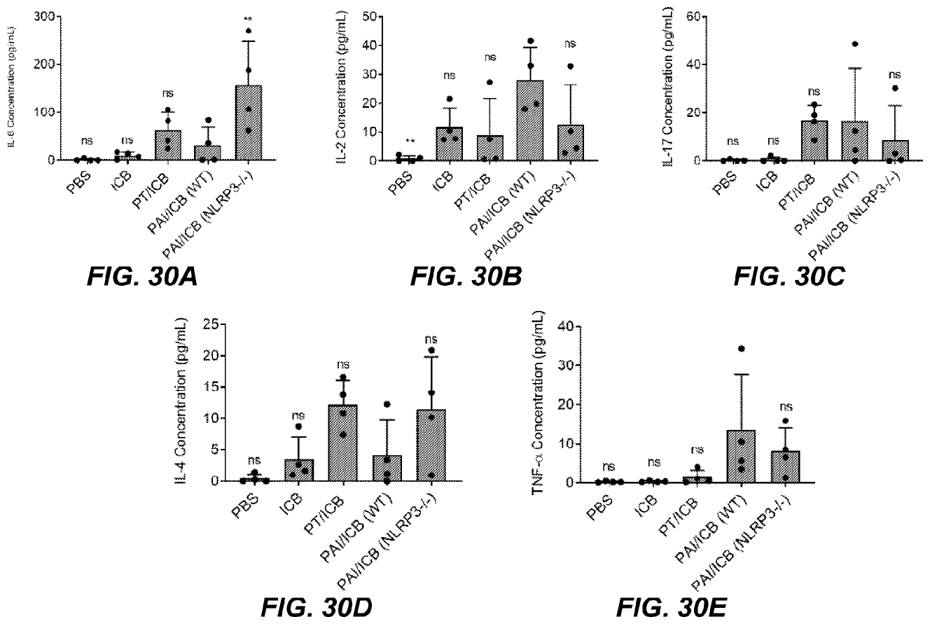

Further to understand the role of each formulation in generation of antigen-specific anti-tumor responses, a parallel study was performed and splenocytes were isolated from treated animals on day 22. Splenocytes were incubated with a neo-antigen cocktail for 48 h and cytokine secretion was measured in the supernatant to analyze for antigen-specific immune response (FIGS. 7D-7G). It was observed that, compared with other treatment groups, only the PAI formulations induced detectable levels of the key immunostimulatory cytokines IFN-g (FIG. 7D) and the apoptosis inducing protease Granzyme-B (FIG. 7E) in all the samples. In comparison, splenocytes from animals receiving ICB therapy alone did not include any detectable levels of IFN-g or Granzyme-B while PT treated animals were observed to induce detectable levels of IFN-g or Granzyme-B in only 50% of samples. Moreover, compared to ICB therapy, the PAI treated wild type (WT) mice did not induce significantly higher levels of the immunosuppressive regulatory cytokine IL-10 (FIG. 7F). The IFN-g/IL-10 ratio in the supernatants was evaluated, which serves as an indicator of prognosis and therapeutic efficacy. Notably, PAI vaccines in WT mice demonstrated twenty-fold higher IFN-g/IL-10 ratio compared to PAI vaccines in NLRP3-deficient mice indicating stronger antigen-specific anti-tumor responses in presence of NLRP3 inflammasome activation in WT mice (FIG. 7G). This results thereby demonstrated highly potent antigen-specific anti-tumor responses against PAI formulated neo-antigen vaccines. Levels of induction of cytokines IL-5, IL-2, IL-17, IL-4, and TNF-α are depicted in FIGS. 30A-30E.

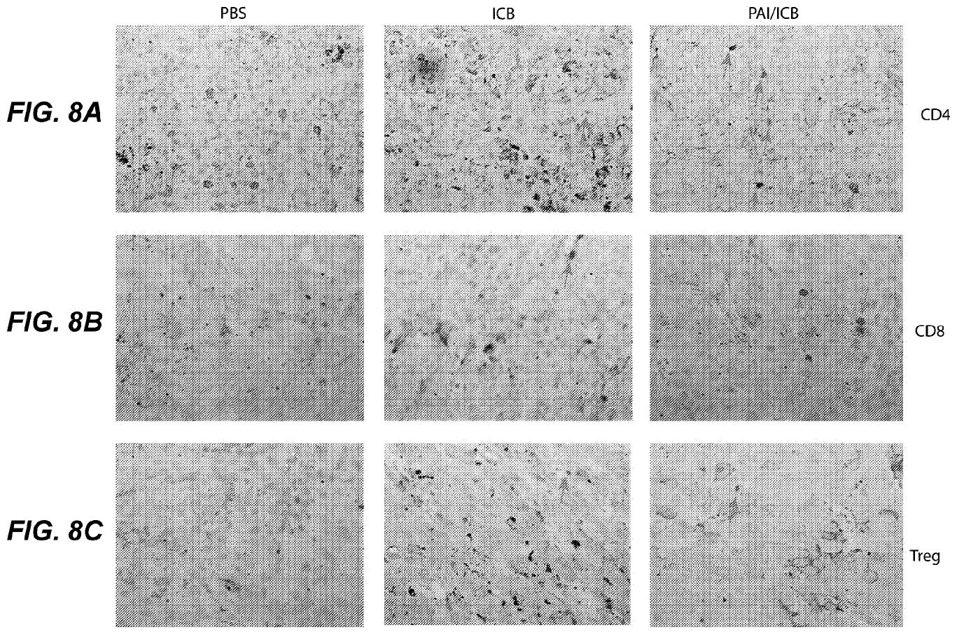

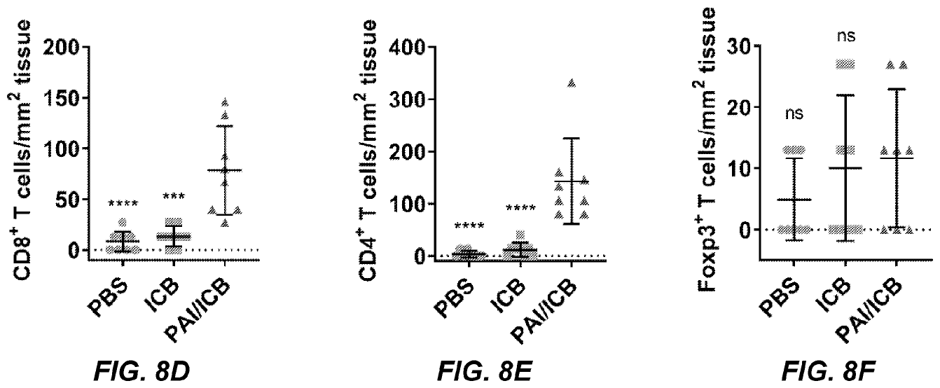

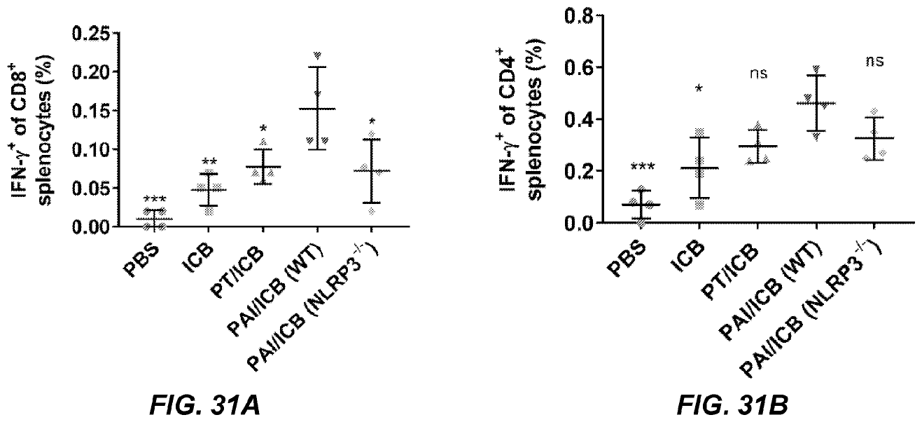

The results from splenocyte analysis demonstrated great promise. With the observation of significantly higher antigen-specific IFN-g secretion by PAI vaccine group compared to the ICB treatment group, the inventors further analyzed for Tumor Infiltrating Leucocytes (TIL) in isolated tumors (day 22). Immunohistochemistry (IHC) staining indicated significantly higher CD4+ and CD8+ TIL in PAI vaccine treated group compared to PBS or ICB treated animals (FIGS. 8A-8C and 31A-31B). No significant difference in FoxP3+ (Regulatory T cells) staining was observed in all the groups. Overall, these data indicated that dual TLR and NLRP3 inflammasome activation by PAI formulation can significantly enhance antitumor efficacy and vaccine-induced protection.

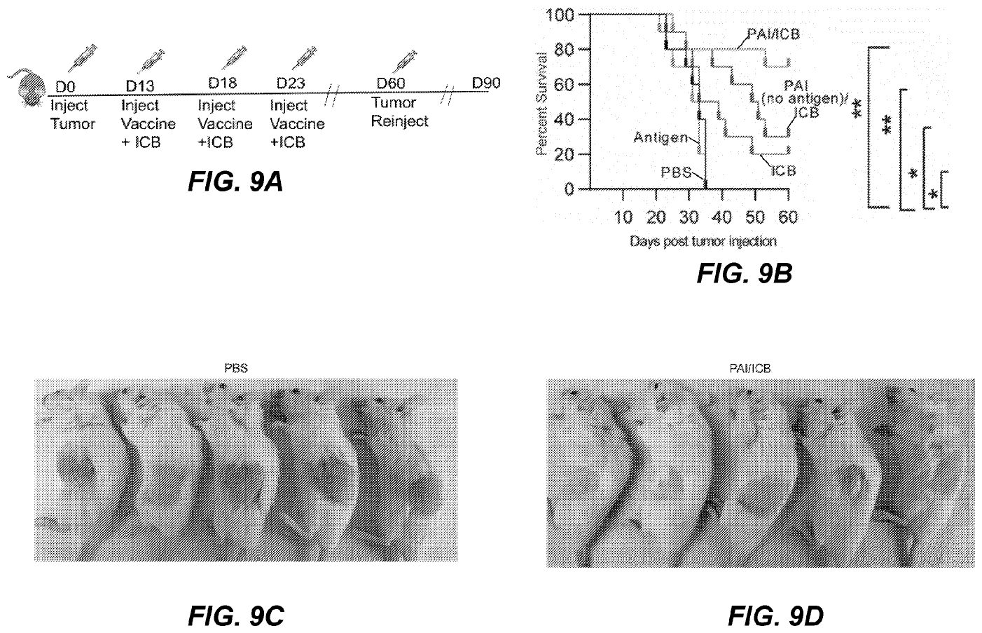

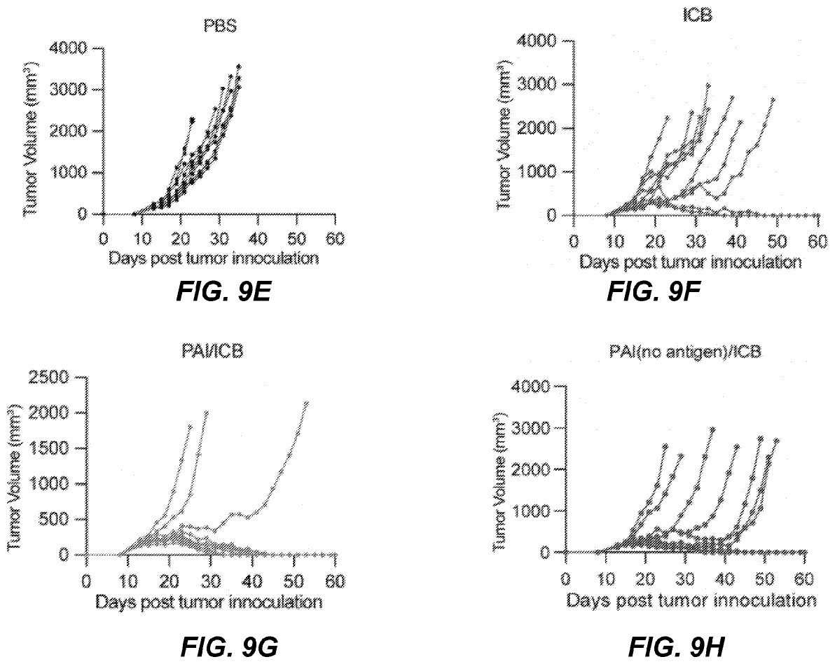

With these exciting findings, the inventors were interested in performing additional studies to analyze efficacy of PAI vaccine formulations against larger and more established tumors. Hence, studies were performed in an aggressive CT26 colon carcinoma model (FIGS. 9A-9G). Here, PAI was similarly formulated with glutamate linker modified CT-26 neoantigen peptides to promote adsorption. Vaccines were administered in mice (n=10) peritumorally on day 13 when tumor volumes reached 150-200 cc followed by two additional boost injections on day 18 and day 23. Alongside PAI vaccine formulation, ICB antibodies were administrated intraperitoneally. Parallel studies were performed with PBS, unadjuvanted antigens and ICB treated mice. Animals were monitored till day 60 when all the treated animals were either observed to be tumor free or were sacrificed due to large tumors (20 mm in any linear dimension). It was observed that PAI vaccines synergized with ICB treatment leading to complete remission of tumors in 70% treated animals on day 60 (FIG. 9B). In contrast, ICB treatment alone or ICB+ antigen only led to complete regression of tumors in just 20% of treated animals. ICB+PAI (without antigens) led to survival in just 30% of treated animals. PBS and antigen treated mice had a median survival of 33 and 32 days respectively. On day 60, all surviving mice in the PAI vaccination group were reinjected with CT26 cells and monitored for any tumor recurrence until day 90. It was observed that these mice completely rejected any new tumor growth, indicating development of immunological memory in the animals. These results thereby further validated the previous observations of enhanced anti-tumor functionality of PAI-vaccine formulations.

### PAI Reduces Off-Target Toxicity

An important attribute of immunomodulatory therapeutics that prevents further clinical translation is unacceptable levels of off-target toxicity. With the remarkable in-vivo antitumor efficacy of PAI vaccines, the inventors next evaluated off-target toxicity of PAI in the CT-26 model. The inventors thereby evaluated for systemic cytokines that are secreted in the blood due to diffusion of immune activators from the site of injection, along with reduction of cellular counts such as WBCs, lymphocytes, and thrombocytes in blood that provide a reflection of toxicity due to diffusion of immune activators. For this study PBS, unlinked agonist formulations, PAI formulations with and without ICB, and ICB were injected on day 13 post tumor injection.

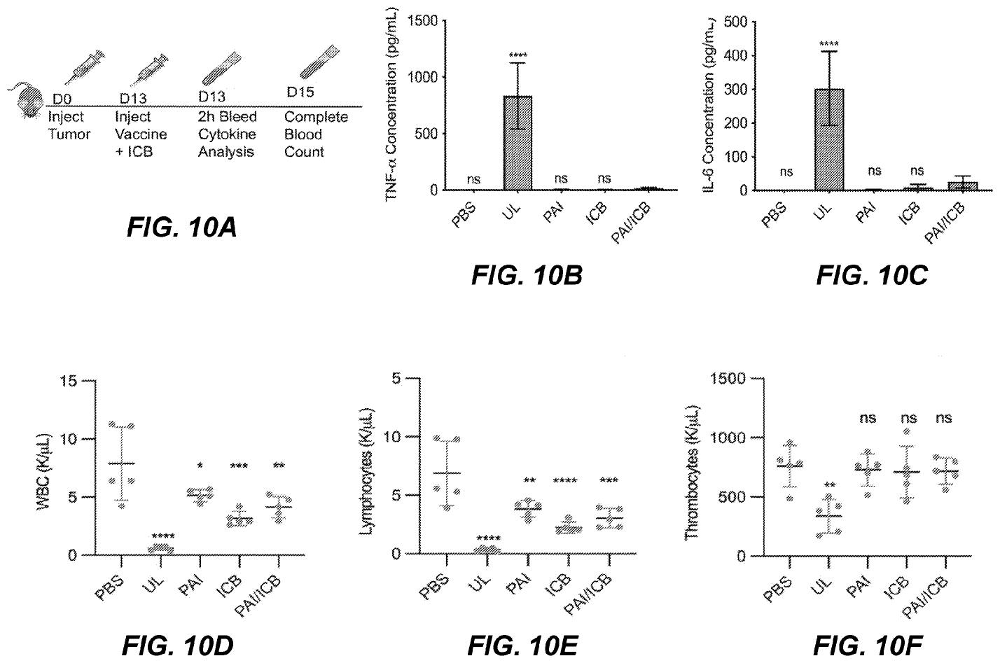

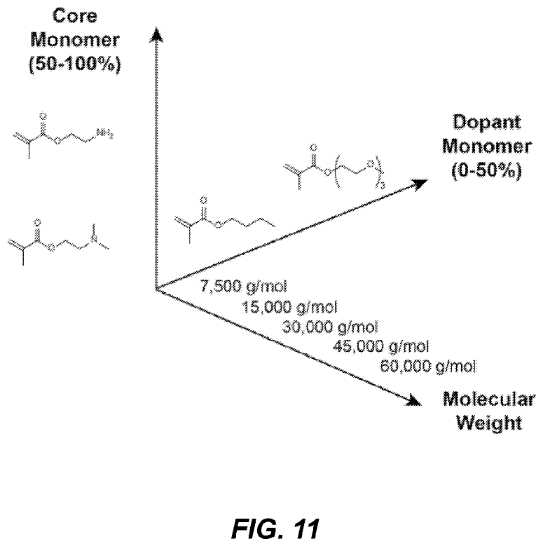

Analysis of serum cytokines (TNF-a and IL-6) two hours post-injection revealed that PAI did not induce significant systemic cytokine secretion (FIGS. 10B and 10C) consistent with the previous observation on enhanced localization of PAI vaccines at the injection site (FIGS. 5A-5C). Notably, when PAI vaccines were combined with ICB therapy, no significant enhancement in levels of systemic cytokines were observed compared to ICB injection alone. In comparison, animals treated with unlinked activator formulation significantly enhanced cytokine secretion in the blood. Consistent with the observation of systemic cytokine secretion, it was observed that PAI formulation induced significantly lower hematological cellular toxicity compared to unlinked activator formulations at 48 h post-injection (FIGS. 10D-10F). Additionally, when combined with ICB the PAI formulation did not significantly enhance hematological toxicity compared to ICB treatment alone. These results thereby suggest that PAI mitigate systemic toxicity compared to treatment with unlinked immune activators.

In conclusion, the inventors designed and synthesized a novel inflammasome activating nano-vaccine platform (PAI) for enhancing efficacy of neo-antigen vaccines. The design incorporated small molecule TLR 7/8 activator 2Bxy along with an inflammasome activating TAT-P6-gwwwg peptide on an amphiphilic carbohydrate scaffold that resulted in self-assembly into stable nanoparticles. PAI demonstrated robust NLRP3-inflammasome activation along with antigen processing in dendritic cells. It also exhibited superior localization at the injection site and draining lymph nodes, resulting in enhanced antigen specific CD8+ T cell responses with reduced systemic cytokine production. A summary of the proposed mechanism of action is provided in FIG. 1C. Briefly, PAI nanovaccines enhance endocytosis by antigen-presenting cells on account of their size ˜50 nm structure. Their secondary structure localizes effects to the injection site. Upon endocytosis, they then activate endosomal TLR7 and induce lysosomal rupture to result in NLRP3 inflammasome activation and cytosolic delivery of neoantigen to afford enhanced antigen presentation on MHC-I. When nanovaccines are injected peritumorally, they induce tumor localized, neoantigen-specific CD8+ T cell responses characterized by secretion of IFN-γ and GNZB to afford tumor clearance.

The described in-vivo vaccination studies, comparing PAI with unlinked activators and checkpoint blockade therapy in multiple tumor models, indicated that PAIs enhanced efficacy of neo-antigen vaccines. PAIs also reduced off-target toxicity compared to unlinked combination of activators. This study thereby assists in understanding the design of materials targeting the NLRP3-inflammasome activation pathway to induce robust protective immune response.

### Example 3—Design and Testing of Inflammasome Activating Polymers

Synthesis of a Library of Polymers that Activate Inflammasomes

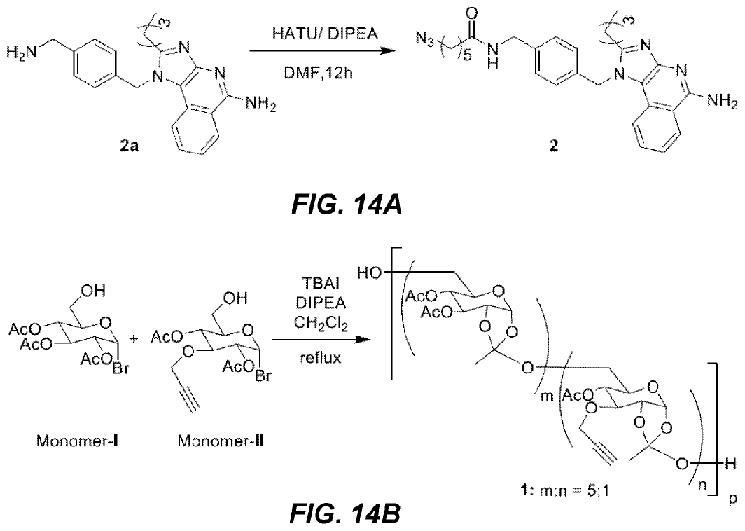

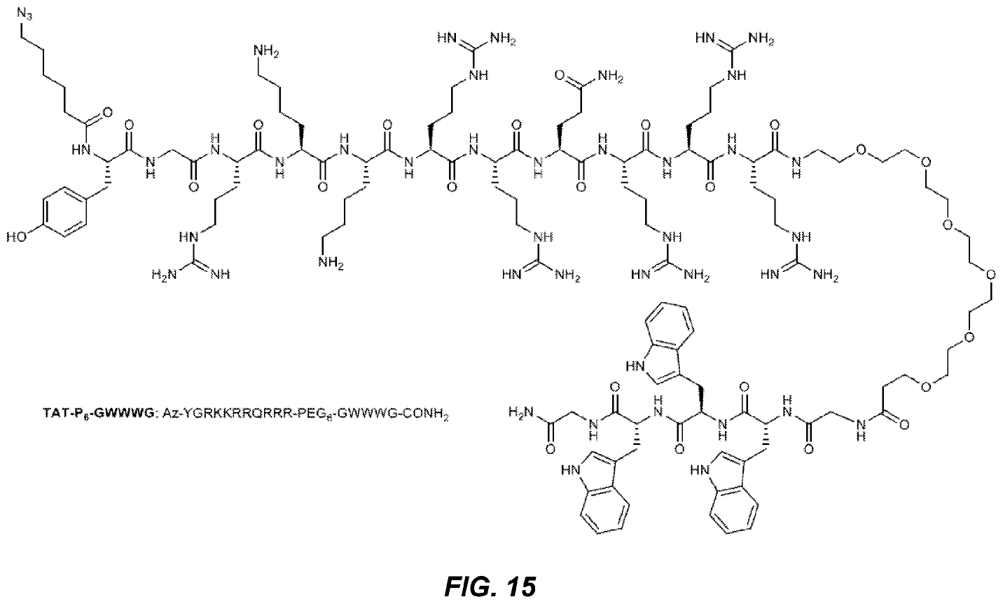

In designing a polymeric library of inflammasome activating polymers, the inventors sought to employ an approach that was (1) able to capitulate lysosomal rupture in a reproducible manner, (2) tunable over a broad domain space, (3) easily synthesized using high throughput techniques, and (4) free of toxic contaminants (such as CuBr or NaN3). Given these considerations, reversible addition-fragmentation chain-transfer (RAFT) polymerization was selected. Given previous work demonstrating that amine-based polymers could facilitate endosomolysis, two monomers (DMAEMA and AEMA) which contained primary and tertiary amines were selected to comprise the bulk (50-100%) of the backbone. Two monomers (TEGMA and BMA) which could further tune the polarity and osmolarity of the polymer backbone were then selected as dopant (0-50%) species. As shown in the design scheme in FIG. 1A and FIGS. 14A and 14B, polymers were prepared at five target molecular weights (7.5, 15, 30, 45, and 60 kg/mol) on a 100 mg scale to generate a library of 110 polymers. For synthesis, AEMA was protected using the Boc protection scheme, and all polymers were treated with trifluoroacetic acid after RAFT to remove the Boc and generate a uniform positive charge on the polymers. Polymers were characterized by size exclusion chromatography (SEC) in DMF and 1H-NMR in D20. As a quality control, all polymers were required to be within 30% of the target molecular weight, 7.5% of the target mass composition, and to be monodisperse (containing a uniform SEC trace with PDI<1.7).

### Characterization of Toxicity and Inflammasome Activation

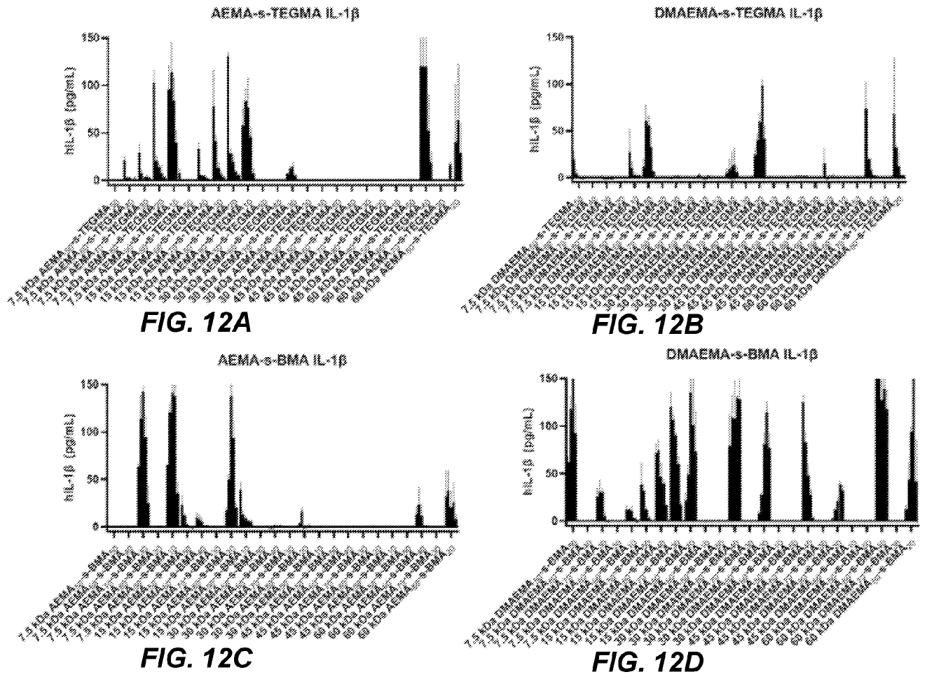

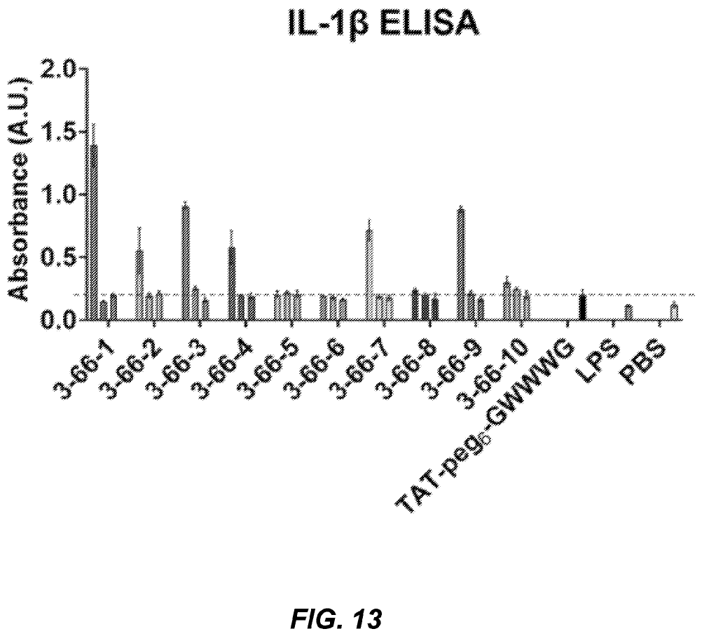

After synthesizing the library, polymers were initially characterized in their ability to induce NLRP3 inflammasome activation (characterized by downstream IL-1β secretion) and pyroptotic cell death (characterized by lactate dehydrogenase [LDH] release). To do so, the THP-1 human monocyte cell line was employed as described previously. 200,000 cells were primed with LPS for 3 h and subsequently treated with the polymers at 100, 50, 25, 12.5, and 6.25 μg/mL for 5 h. The media was then collected and assayed by IL-1β ELISA (FIGS. 12 and 13) and enzymatic LDH-mediated tetrazolium reduction. It was found that several parameters played a role in lysosomal rupture; indeed, high molecular weight polymers comprised of DMAEMA and BMA as well as low molecular weight polymers comprised of AEMA and BMA were superior in activating the inflammasome, particularly when containing a larger weight fraction of BMA. It was hypothesized that the BMA provided the necessary hydrophobicity for the polymers to insert in the lysosomal membrane and catalyze its rupture upon protonation, while the differential effects of DMAEMA and AEMA might have resulted from differences in endocytosis and cellular trafficking of the various components. Given these results, a secondary assay was conducted to elucidate the kinetics and extent of lysosomal rupture mediated by the polymers to determine whether these were responsible for the observed effects.

Tables 1 and 2 provide lists of synthesized polymers and certain characteristics.

TABLE 1

| Target | | | | | | % |
|---|---|---|---|---|---|---|
| molecular | | | Target % | | | Monomer 1 |
| weight | Monomer 1 | Monomer 2 | Monomer 1 | Mn | Ð | (by NMR) |
| 7,500 | AEMA | BMA | 60 | 6,800 | 1.19 | 62% |
| 7,500 | AEMA | BMA | 70 | 7,500 | 1.22 | 76% |
| 7,500 | AEMA | BMA | 80 | 7,400 | 1.25 | 83% |
| 7,500 | AEMA | BMA | 90 | 8,800 | 1.14 | 91% |
| 7,500 | AEMA | N/A | 100 | 8,000 | 1.18 | 100% |
| 7,500 | AEMA | TEGMA | 60 | 8,800 | 1.19 | 64% |
| 7,500 | AEMA | TEGMA | 70 | 6,700 | 1.26 | 76% |
| 7,500 | AEMA | TEGMA | 80 | 9,600 | 1.16 | 79% |
| 7,500 | AEMA | TEGMA | 90 | 7,400 | 1.18 | 91% |
| 7,500 | DMAEMA | BMA | 50 | 6,000 | 1.37 | 45% |
| 7,500 | DMAEMA | BMA | 70 | 5,900 | 1.37 | 69% |
| 7,500 | DMAEMA | BMA | 90 | 5,400 | 1.37 | 91% |
| 7,500 | DMAEMA | N/A | 100 | 8,700 | 1.33 | 100 |
| 7,500 | DMAEMA | TEGMA | 50 | 5,600 | 1.37 | 51% |
| 7,500 | DMAEMA | TEGMA | 70 | 5,700 | 1.39 | 66% |
| 7,500 | DMAEMA | TEGMA | 90 | 5,900 | 1.39 | 86% |
| 15,000 | AEMA | BMA | 50 | 12,700 | 1.17 | 54% |
| 15,000 | AEMA | BMA | 60 | 12,700 | 1.17 | 63% |
| 15,000 | AEMA | BMA | 70 | 18,800 | 1.17 | 68% |
| 15,000 | AEMA | BMA | 80 | 14,700 | 1.17 | 81% |
| 15,000 | AEMA | BMA | 90 | 15,700 | 1.14 | 91% |
| 15,000 | AEMA | N/A | 100 | 14,600 | 1.25 | 100% |
| 15,000 | AEMA | TEGMA | 60 | 16,600 | 1.17 | 63% |
| 15,000 | AEMA | TEGMA | 70 | 13,300 | 1.21 | 75% |
| 15,000 | AEMA | TEGMA | 80 | 17,600 | 1.18 | 79% |
| 15,000 | AEMA | TEGMA | 90 | 15,000 | 1.18 | 89% |
| 15,000 | DMAEMA | BMA | 50 | 12,300 | 1.28 | 52% |
| 15,000 | DMAEMA | BMA | 60 | 11,500 | 1.35 | 65% |
| 15,000 | DMAEMA | BMA | 70 | 11,500 | 1.39 | 70% |
| 15,000 | DMAEMA | BMA | 80 | 13,200 | 1.27 | 79% |
| 15,000 | DMAEMA | BMA | 90 | 13,000 | 1.32 | 93% |
| 15,000 | DMAEMA | N/A | 100 | 19,100 | 1.26 | 100% |
| 15,000 | DMAEMA | TEGMA | 50 | 11,400 | 1.36 | 50% |
| 15,000 | DMAEMA | TEGMA | 70 | 11,500 | 1.37 | 64% |
| 30,000 | AEMA | BMA | 50 | 31,800 | 1.20 | 44% |
| 30,000 | AEMA | BMA | 60 | 23,200 | 1.19 | 62% |
| 30,000 | AEMA | BMA | 70 | 27,900 | 1.36 | 65% |
| 30,000 | AEMA | BMA | 80 | 25,100 | 1.2 | 83% |
| 30,000 | AEMA | BMA | 90 | 34,700 | 1.22 | 92% |
| 30,000 | AEMA | N/A | 100 | 25,000 | 1.16 | 100% |
| 30,000 | AEMA | TEGMA | 50 | 32,400 | 1.22 | 57% |
| 30,000 | AEMA | TEGMA | 60 | 22,300 | 1.24 | 66% |
| 30,000 | AEMA | TEGMA | 70 | 33,700 | 1.22 | 74% |
| 30,000 | AEMA | TEGMA | 80 | 23,700 | 1.25 | 82% |
| 30,000 | AEMA | TEGMA | 90 | 34,400 | 1.26 | 91% |
| 30,000 | DMAEMA | BMA | 60 | 23,600 | 1.31 | 62% |
| 30,000 | DMAEMA | BMA | 80 | 24,000 | 1.37 | 83% |
| 30,000 | DMAEMA | BMA | 90 | 30,700 | 1.49 | 86% |
| 30,000 | DMAEMA | N/A | 100 | 36,900 | 1.35 | 100% |
| 30,000 | DMAEMA | TEGMA | 50 | 32,000 | 1.47 | 50% |
| 30,000 | DMAEMA | TEGMA | 70 | 34,400 | 1.43 | 68% |
| 30,000 | DMAEMA | TEGMA | 80 | 21,100 | 1.53 | 74% |
| 30,000 | DMAEMA | TEGMA | 90 | 30,800 | 1.53 | 85% |
| 45,000 | AEMA | BMA | 50 | 42,200 | 1.28 | 42% |
| 45,000 | AEMA | BMA | 80 | 41,400 | 1.40 | 80% |
| 45,000 | AEMA | BMA | 90 | 41,900 | 1.39 | 92% |
| 45,000 | AEMA | N/A | 100 | 43,700 | 1.23 | 100% |
| 45,000 | AEMA | TEGMA | 50 | 41,500 | 1.34 | 57% |
| 45,000 | AEMA | TEGMA | 70 | 38,900 | 1.47 | 72% |
| 45,000 | AEMA | TEGMA | 80 | 46,500 | 1.29 | 81% |
| 45,000 | AEMA | TEGMA | 90 | 46,800 | 1.32 | 91% |
| 45,000 | DMAEMA | BMA | 50 | 39,600 | 1.46 | 48% |
| 45,000 | DMAEMA | BMA | 60 | 34,400 | 1.32 | 61% |
| 45,000 | DMAEMA | BMA | 70 | 38,500 | 1.58 | 67% |
| 45,000 | DMAEMA | BMA | 80 | 32,000 | 1.47 | 82% |
| 45,000 | DMAEMA | BMA | 90 | 38,200 | 1.66 | 91% |
| 45,000 | DMAEMA | N/A | 100 | 40,000 | 1.51 | 100% |
| 45,000 | DMAEMA | TEGMA | 50 | 42,900 | 1.65 | 51% |
| 45,000 | DMAEMA | TEGMA | 70 | 42,800 | 1.66 | 68% |
| 45,000 | DMAEMA | TEGMA | 90 | 42,500 | 1.57 | 82% |
| 60,000 | AEMA | BMA | 60 | 49,500 | 1.25 | 60% |
| 60,000 | AEMA | BMA | 70 | 53,500 | 1.35 | 67% |
| 60,000 | AEMA | BMA | 80 | 52,900 | 1.26 | 82% |
| 60,000 | AEMA | BMA | 90 | 46,300 | 1.58 | 88% |
| 60,000 | AEMA | N/A | 100 | 50,900 | 1.23 | 100% |
| 60,000 | AEMA | TEGMA | 50 | 54,000 | 1.31 | 57% |
| 60,000 | AEMA | TEGMA | 60 | 49,300 | 1.26 | 66% |
| 60,000 | AEMA | TEGMA | 70 | 59,100 | 1.31 | 72% |
| 60,000 | AEMA | TEGMA | 80 | 47,600 | 1.37 | 82% |
| 60,000 | AEMA | TEGMA | 90 | 64,600 | 1.27 | 90% |
| 60,000 | DMAEMA | BMA | 60 | 48,700 | 1.44 | 67% |
| 60,000 | DMAEMA | BMA | 70 | 47,900 | 1.65 | 67% |
| 60,000 | DMAEMA | BMA | 90 | 63,900 | 1.38 | 91% |
| 60,000 | DMAEMA | N/A | 100 | 49,500 | 1.65 | 100% |
| 60,000 | DMAEMA | TEGMA | 60 | 47,200 | 1.57 | 59% |

TABLE 2

| | Target | | | | | |
|---|---|---|---|---|---|---|
| | molecular | | | Mn | Ð | Mn |
| ID | weight | Monomer 1 | Monomer 2 | (GPC) | (GPC) | (UV-VIS) |
| 3-66-1 | 20,000 | AMEA (100%) | N/A | 13,900 | 1.18 | 8,000 ± 200 |
| 3-66-2 | 40,000 | AMEA (100%) | N/A | 25,700 | 1.25 | 18,800 ± 5,900 |
| 3-66-3 | 60,000 | AMEA (100%) | N/A | 30,300 | 1.16 | 21,200 ± 1,200 |
| 3-66-4 | 80,000 | AMEA (100%) | N/A | 44,100 | 1.16 | 34,200 ± 25,900 |
| 3-66-5 | 10,000 | AMEA (80%) | TEGMA (20%) | 5,000 | 1.24 | 4,800 ± 2,400 |
| 3-66-6 | 10,000 | AMEA (60%) | TEGMA (40%) | 4,2000 | 1.26 | 4,400 ± 2,100 |
| 3-66-7 | 30,000 | AMEA (80%) | TEGMA (20%) | 14,400 | 1.16 | 10,400 ± 3,500 |
| 3-66-8 | 30,000 | AMEA (60%) | TEGMA (40%) | 12,000 | 1.19 | 8,700 ± 3,900 |
| 3-66-9 | 60,000 | AMEA (80%) | TEGMA (20%) | 26,700 | 1.18 | 17,500 ± 5,100 |
| 3-66-10 | 60,000 | AMEA (60%) | TEGMA (40%) | 22,700 | 1.17 | 14,900 ± 4,700 |

### Materials and Methods

### Materials

Unless otherwise noted, all materials were purchased from Sigma Aldrich, Thermo Scientific, or VWR and used as received. THP-1 cells were obtained from ATCC and cultured in RPMI with 10% heat inactivated FBS at 37° C. and 5% CO2. TAT-peg6-GWWWG was synthesized by SPPS using a previously reported procedure.

### Synthesis of 2-(N-(tert-butoxycarbonyl)amino) ethyl methacrylate

The title compound was prepared as reported previously with minor changes.²³ Briefly, 60.0 g di-tert-butyl dicarbonate was dissolved in 100 mL DCM and placed in a 250 mL flask containing a stir bar, placed under an addition funnel, and sealed under Argon. The flask was placed in an ice bath, and 25.0 mL 2-aminoethan-1-ol was added to the addition funnel and added dropwise under rapid stirring. After addition was complete, the reaction was warmed to room temperature and stirred for 16 h. The reaction was then extracted sequentially with 100 mL 0.1 M NaOH, 100 mL H₂O, and 100 mL brine, and the organic phase was collected and dried over MgSO₄. The solvent was removed under reduced pressure to crude obtain N-(tert-Butoxycarbonyl) ethanolamine which was used without further purification. 10.0 g of N-(tert-Butoxycarbonyl) ethanolamine was then added to a flame dried flask with 8.0 mL triethylamine and a stir bar, dissolved in 100 mL dry DCM, and sealed under argon. The flask was placed in an ice bath and 9.0 mL methacryloyl chloride in 50 mL dry DCM was added over 30 min with rapid stirring. Upon complete addition, the reaction was warmed to room temperature and stirred for 16 h. The reaction was then quenched by addition of 100 mL 0.1 M NaOH, and the organic phase was washed with 3×100 mL H₂O and 100 mL brine prior to drying over MgSO₄. The crude product was loaded onto silica and separated by flash chromatography (3:2 Hexanes:EtOAc). The product was concentrated under reduced pressure and recrystallized from 1:1 Hexanes:DCM to obtain 2-(N-(tert-butoxycarbonyl)amino) ethyl methacrylate as a white crystalline solid. 1H-NMR: 6.13 (s, 1H), 5.59 (s, 1H), 4.79 (br s, 1H), 4.21 (t, 2H), 3.45 (q, 2H), 1.95 (s, 3H), 1.45 (s, 9H).

### General Strategy for Synthesis of Polymers

Polymers were prepared on a 100 mg scale using an Explorer (Unchained Labs) automated process chemistry instrument. 2-(N-(tert-butoxycarbonyl)amino) ethyl methacrylate (BocAEMA), 4,4′-Azobis(4-cyanovaleric acid) (ACVA), and 4-((((2-Carboxyethyl)thio) carbonothioyl)thio)-4-cyanopentanoic acid (CTA) were recrystallized prior to use. Butyl methacrylate (BMA) and 2-(Dimethylamino)ethyl methacrylate (DMAEMA) were distilled under reduced pressure prior to use. BMA, DMAEMA, and triethylene glycol methyl ether methacrylate (TEGMA) were passed through a column of activated basic alumina prior to use. BocAEMA, DMAEMA, BMA, and TEGMA were dissolved at 250 mg/mL in dry DMF, and ACVA and CTA were dissolved at 10 mg/mL in dry DMF. Using the Explorer instrument under an inert nitrogen atmosphere, all reagents were added in appropriate ratios to 2 mL vessels and diluted to a final volume of 1 mL in dry DMF. The vessels were then heated to 72° C. and shaken at 1500 rpm. After 24 h, the vessels were cooled and exposed to air to quench the reaction. 25 μL aliquots were collected for size exclusion chromatography. The amine-based polymers were protonated by addition of 500 μL TFA, precipitated into 50 mL of 1:1 diethyl ether:hexanes, and collected by centrifugation. All polymers were then treated with 2 mL TFA for 1 h to remove N-(tert-butoxycarbonyl) protecting groups or as a control treatment. After 1 h, the polymers were re-precipitated into 50 mL of 1:1 diethyl ether:hexanes, collected by centrifugation, and taken up in 5 mL dH₂O. The polymers were dialyzed sequentially against 0.5 M NaCl and dH₂O and then lyophilized to obtain the target materials as white or pink aerogels. 1H-NMR and SEC characterization is provided in the Supplementary Information.

### Polymer Characterization

GPCs was conducted in DMF with 0.01 M LiBr additive at 50° C. using a Tosoh EcoSEC system equipped in series with Tosoh SuperAW3000 and Tosoh SuperAW4000 columns. Polymer molecular weight was calculated relative to PMMA standards. 1H-NMR was conducted at 400 MHz on a Bruker DRX instrument equipped with a BBO probe using Topspin 1.3 and analyzed using MestreNova software.

### Inflammasome Activation and Cytotoxicity Assay

THP-1 cells were cultured in RPMI, 10% HI-FBS, 100 U/mL penicillin, and 100 μg/mL streptomycin at 5% CO2 and 37° C. For IL-1β quantitation, cells were passaged and plated at 180,000 cells/well in a 96 well plate and treated with 100 EU/mL LPS-EB (InvivoGen). After 3 h, cells were washed with PBS, resuspended in media, and treated with a final concentration of 100, 50, 25, 12.5, or 6.25 μg/mL of the polymers or controls. After 5 h, the supernatant was collected and subjected to human IL-1β ELISA Kit (Thermo Scientific) and CyQUANT LDH Cytotoxicity Assay (Thermo Fisher) according to the manufacturer's procedures.

### Characterization of Lysosomal Rupture

To measure lysosomal rupture capitulated by the polymers over the course of several hours, live cell fluorescent imaging using the acridine orange (AO) stain was employed. AO is a cell permeable dye which accumulates in in-tact lysosomes and fluoresces red on account of pH-induced dimerization. Upon lysosomal rupture, the AO is released into the cytosol where its monomer fluoresces green, thereby providing a readout of fluorescent intensity. 20,000 LPS-primed THP-1 cells are incubated with 1 μg/mL AO for 30 min and then treated with the polymers at 100, 50, 25, 12.5, and 6.25 μg/mL; upon addition, the polymers are placed in a IncuCyte S3 high throughput fluorescent imager which is housed within a cell culture incubator (37° C., 5% CO2) and imaged in 15 min intervals to observe lysosomal rupture under homeostatic conditions.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The references cited herein, to the extent that they provide exemplary procedural or other details supplementary to those set forth herein, are specifically incorporated herein by reference.

What is claimed is:

1. A polymer of formula (I): 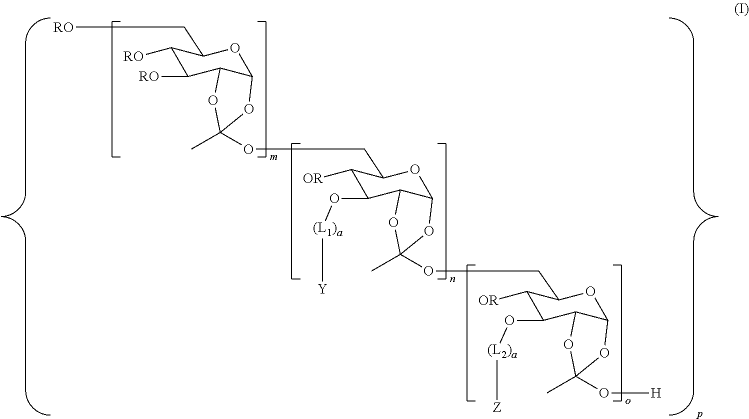
wherein
each R is independently H, alkyl, or acyl;
m is an integer ranging from 0 to 10;
n is an integer ranging from 0 to 10;
o is an integer ranging from 0 to 10;
p is an integer ranging from 1 to 500;
L₁ is a first linker;
Z comprises a first polypeptide;
L₂ is a second linker;
Y comprises an adjuvant;
a is 0 or 1; and
b is 0 or 1;
wherein n+o≥1.

2. The polymer of claim 1, wherein L₁ and L₂ each independently comprise at least one of a maleimide moiety, a polyethylene glycol (PEG) moiety, and a triazole moiety.

3. The polymer of claim 1 or 2, wherein n is 0.

4. The polymer of claim 1 or 2, wherein o is 0.

5. The polymer of claim 1 or 2, wherein n is equal to o.

6. The polymer of claim 1 or 2, wherein the ratio of m:(n+o) is 5:1.

7. The polymer of any of claims 1-6, wherein Z is of formula (II): 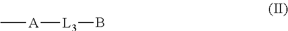
wherein A is the first polypeptide, L₃ is a third linker, and B is a second polypeptide.

8. The polymer of claim 7, wherein A is an agent capable of rupturing an endosome or a lysosome.

9. The polymer of claim 7 or 8, wherein A is a cell penetrating peptide.

10. The polymer of claim 9, wherein the cell penetrating peptide is a TAT peptide, penetratin, transportan, MAP, Pep-1, Pept 1, Pept 2, IVV-14, pVEC, HRSV, or polyarginine.

11. The polymer of claim 9 or 10, wherein the cell penetrating peptide comprises a sequence from an HIV TAT protein.

12. The polymer of claim 11, wherein the sequence is a sequence from amino acids 45-65 of HIV-1 TAT protein.

13. The polymer of claim 12, wherein the sequence is amino acids 48-60 of HIV-1 TAT protein (SEQ ID NO:1).

14. The polymer of claim 12, wherein the sequence is amino acids 47-57 of HIV-1 TAT protein (SEQ ID NO:2).

15. The polymer of any of claims 7-14, wherein B is a hydrophobic endosomal escape peptide.

16. The polymer of claim 15, wherein the hydrophobic endosomal escape peptide has the sequence GWWWG (SEQ ID NO: 3), GFWFG (SEQ ID NO: 4), or GWWG (SEQ ID NO: 5).

17. The polymer of any of claims 7-16, wherein L₃ is a polyethylene glycol (PEG) linker, a N-(2-hydroxypropyl)-methacrylamide (HPMA) linker, a PEG-methylacrylamide (PEGMA) linker, a succinimide linker, a maleimide linker, a polyamide linker, a polyester linker, or a bifunctional or trifunctional linker comprising a combination of the aforementioned linkers comprising c monomeric units.

18. The polymer of claim 17, wherein c is between 2 and 20.

19. The polymer of claim 18, wherein c is 6.

20. The polymer of any of claims 1-19, wherein Z comprises an inflammasome activator.

21. The polymer of claim 20, wherein the inflammasome activator is capable of activating an NLRP3 inflammasome.

22. The polymer of claim 20 or 21, wherein the polymer is capable of stimulating IL-1β production in a subject.

23. The polymer of any of claims 20-22, wherein the polymer is capable of stimulating activation and/or proliferation of CD4+ T cells in a subject.

24. The polymer of any of claims 1 to 23, wherein the adjuvant is a TLR agonist.

25. The polymer of claim 24, wherein the TLR agonist is a TLR7 agonist or a TLR4 agonist.

26. The polymer of claim 24, wherein the TLR agonist is 2Bxy or a functionalized 2Bxy.

27. The polymer of any of claims 1-26, wherein the polymer further comprises an additional adjuvant.

28. The polymer of claim 27, wherein the additional adjuvant is attached to the same polymer residue as the first polypeptide.

29. The polymer of claim 1, wherein the polymer is of formula (III):

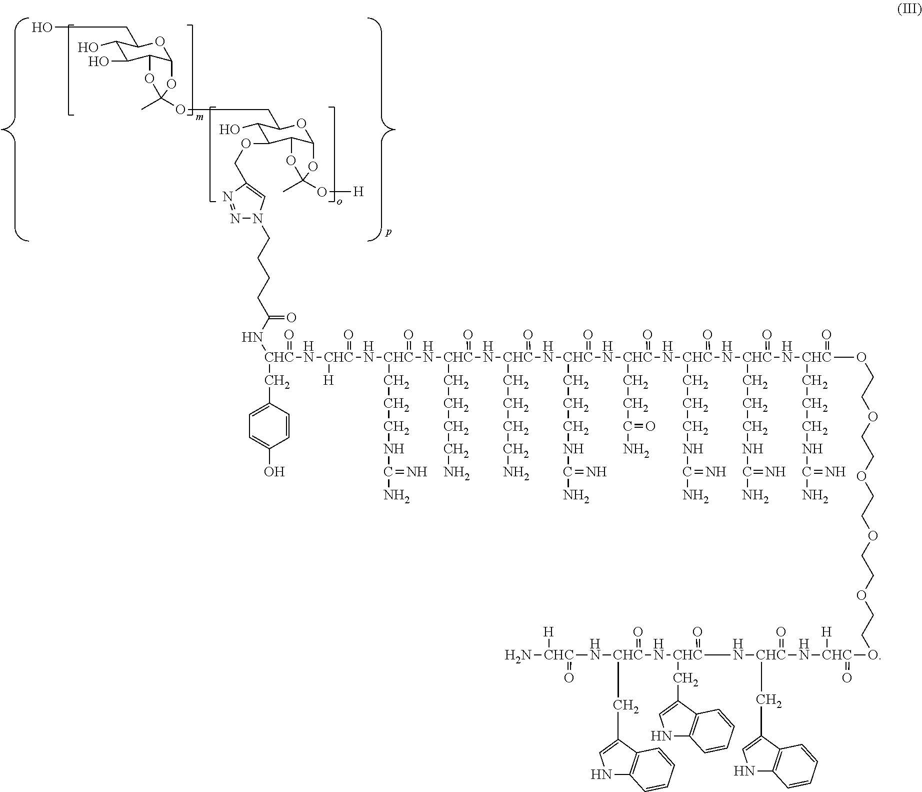

30. The polymer of any of claims 1-29, wherein the polymer is capable of stimulating activation and/or proliferation of CD8+ T cells in a subject.

31. The polymer of any of claims 1-30, wherein at least one R group is an acetyl group.

32. The polymer of claim 31, wherein the polymer is of formula (IV):

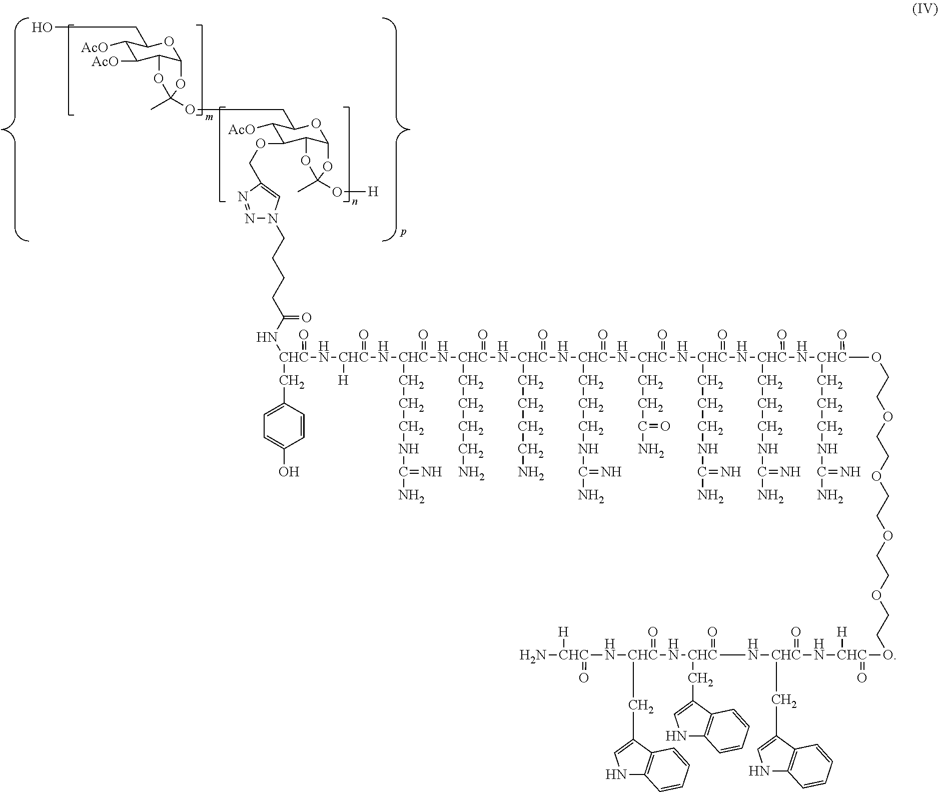

33. The polymer of any of claims 1-32, wherein the polymer is of formula (V):

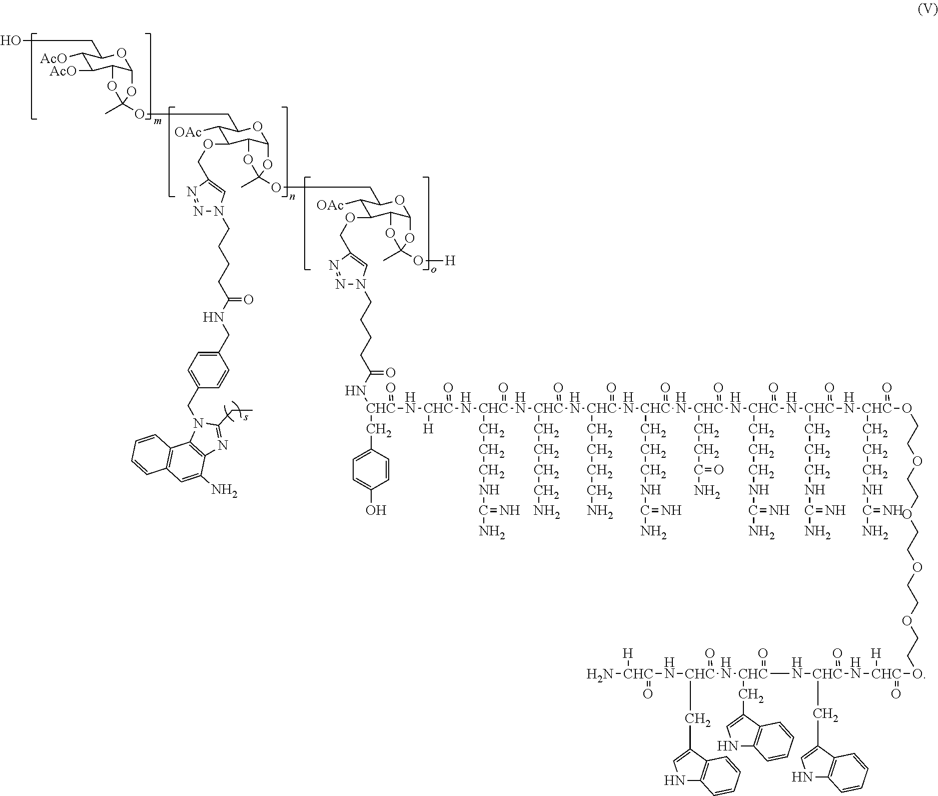

34. The polymer of any of claims 1-32, wherein the polymer is of formula (VI):

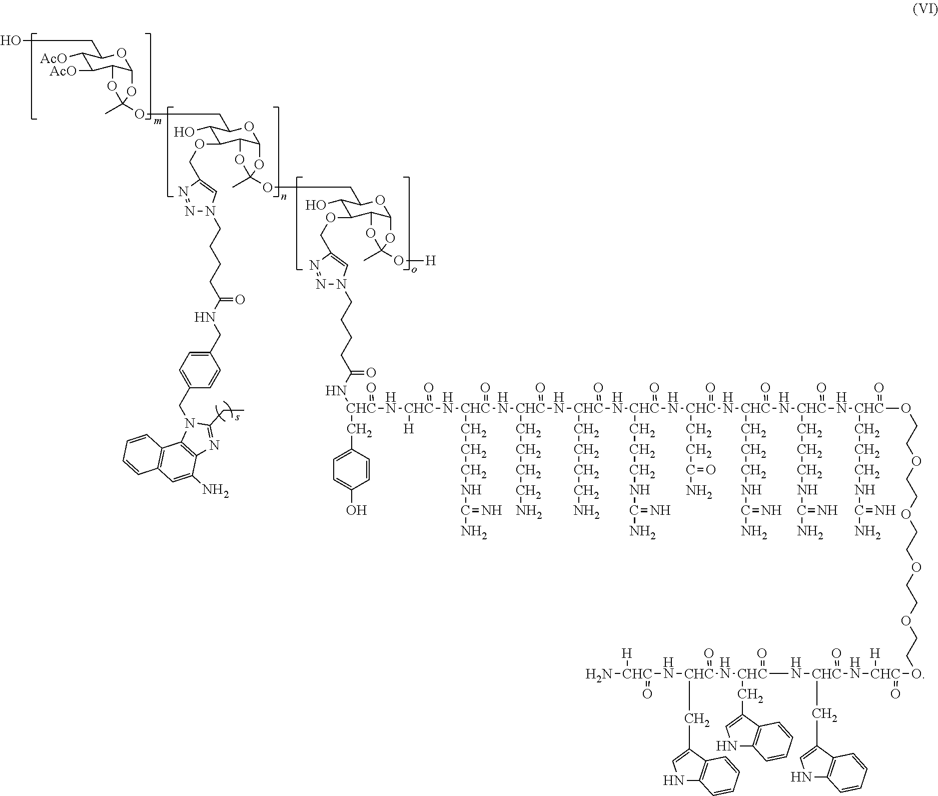

35. A nanoparticle comprising the polymer of any of claims 1-34.

36. A method of stimulating an immune response to an antigen, the method comprising administering to a subject a pharmaceutical composition comprising the antigen and an effective amount of the polymer of any of claims 1-34 or the nanoparticle of claim 35.

37. The method of claim 36, wherein the pharmaceutical composition further comprises an additional adjuvant.

38. The method of claim 37, wherein the additional adjuvant is a toll-like receptor (TLR) agonist.

39. The method of claim 38, wherein the TLR agonist is a TLR4 agonist.

40. The method of claim 39, wherein the TLR4 agonist is lipopolysaccharide (LPS), monophosphoryl lipid A (MPLA), Fimbriae H protein (FimH), Microfilarial protein (MfP), or a synthetic TLR4 agonist including a phosphorylated hexaacyl disaccharide (PHAD), an aminoalkyl glucosaminide phosphate (AGP), an OMV with attenuated endotoxicity (fmOMV), E6020, or a combination thereof.

41. The method of any of claims 36-40, wherein the pharmaceutical composition further comprises a pharmaceutically acceptable excipient.

42. A method of improving an efficacy of a vaccine, the method comprising administering to a subject a pharmaceutical composition comprising the vaccine and an effective amount of the polymer of any of claims 1-34 or the nanoparticle of claim 35.

43. The method of claim 42, wherein the vaccine comprises an antigen.

44. The method of claim 42 or 43, wherein the vaccine further comprises an additional adjuvant.

45. The method of claim 44, wherein the additional adjuvant is a toll-like receptor (TLR) agonist.

46. The method of claim 45, wherein the TLR agonist is a TLR4 agonist.

47. The method of claim 46, wherein the TLR4 agonist is lipopolysaccharide (LPS), monophosphoryl lipid A (MPLA), Fimbriae H protein (FimH), Microfilarial protein (MfP), or a synthetic TLR4 agonist including a phosphorylated hexaacyl disaccharide (PHAD), an aminoalkyl glucosaminide phosphate (AGP), an OMV with attenuated endotoxicity (fmOMV), E6020, or a combination thereof.

48. The method of any of claims 42-47, wherein the pharmaceutical composition further comprises a pharmaceutically acceptable excipient.

49. A method of stimulating activation and/or proliferation of CD8+ T cells in a subject comprising administering to the subject an effective amount of the polymer of any of claims 1-34 or the nanoparticle of claim 35.

50. A method of stimulating CD4+ T cell activation or proliferation in a subject comprising administering to the subject an effective amount of the polymer of any of claims 1-34 or the nanoparticle of claim 35.

51. The method of claim 49 or 50, further comprising administering an antigen to the subject.

52. The method of any of claims 49-51, further comprising administering an additional adjuvant to the subject.

53. The method of claim 52, wherein the additional adjuvant is a toll-like receptor (TLR) agonist.

54. The method of claim 53, wherein the TLR agonist is a TLR4 agonist.

55. The method of claim 54, wherein the TLR4 agonist is lipopolysaccharide (LPS), monophosphoryl lipid A (MPLA), Fimbriae H protein (FimH), Microfilarial protein (MfP), or a synthetic TLR4 agonist including a phosphorylated hexaacyl disaccharide (PHAD), an aminoalkyl glucosaminide phosphate (AGP), an OMV with attenuated endotoxicity (fmOMV), E6020, or a combination thereof.

56. A method of making a vaccine comprising generating a pharmaceutical composition comprising an antigen and an effective amount of the polymer of any of claims 1-34 or the nanoparticle of claim 35.

57. The method of claim 56, wherein the pharmaceutical composition further comprises an additional adjuvant.

58. The method of claim 57, wherein the additional adjuvant is a toll-like receptor (TLR) agonist.

59. The method of claim 58, wherein the TLR agonist is a TLR4 agonist.

60. The method of claim 59, wherein the TLR4 agonist is lipopolysaccharide (LPS), monophosphoryl lipid A (MPLA), Fimbriae H protein (FimH), Microfilarial protein (MfP), or a synthetic TLR4 agonist including a phosphorylated hexaacyl disaccharide (PHAD), an aminoalkyl glucosaminide phosphate (AGP), an OMV with attenuated endotoxicity (fmOMV), E6020, or a combination thereof.

61. A method for treatment or prevention of cancer, the method comprising administering to a subject an effective amount of a pharmaceutical composition comprising the polymer of any of claims 1-34 or the nanoparticle of claim 35.

62. The method of claim 61, further comprising administering to the subject an additional cancer therapy.

63. The method of claim 62, wherein the additional cancer therapy comprises chemotherapy, radiation therapy, immunotherapy, or a combination thereof.

64. The method of claim 63, wherein the additional cancer therapy comprises immunotherapy.

65. The method of claim 63, wherein the additional cancer therapy is a checkpoint inhibitor therapy.

66. The method of any of claims 61-65, wherein the subject has not been diagnosed with cancer.

67. The method of any of claims 61-65, wherein the subject has been diagnosed with cancer.

68. The method of any of claims 61-67, wherein the subject was previously treated for cancer with a previous therapy.

69. The method of any of claims 61-68, wherein the subject was determined to be resistant to the previous therapy.

70. The method of any of claims 61-69, wherein the pharmaceutical composition is administered to the subject intratumorally.

71. A copolymer comprising:
a) from 10 to 90% by weight of a repeat unit derived from a monomer of formula (VII); 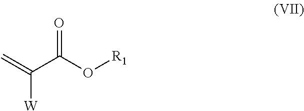
where W is hydrogen or methyl; and
R₁ is an alkyl group having from 1 to 5 carbon atoms, an ether group having an alkyl group of from 1 to 5 carbon atoms between the carboxylate oxygen and the ether oxygen and a methyl or ethyl group attached to the ether oxygen, or an N-terminal alkyl amino group having from 1 to 5 carbon atoms; and
b) from 10 to 90% by weight of a repeat unit derived from a monomer of formula (VIII); 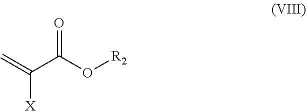
where X is hydrogen or methyl; and
R₂ is an alkyl group having from 1 to 5 carbon atoms, an ether group having an alkyl group of from 1 to 5 carbon atoms between the carboxylate oxygen and the ether oxygen and a methyl or ethyl group attached to the ether oxygen, or an N-terminal alkyl amino group having from 1 to 5 carbon atoms.

72. The copolymer of claim 71, wherein R₁ is an N-terminal alkyl amino group having from 1 to 5 carbon atoms, wherein the amino group is substituted with a methyl group, two methyl groups, or a tert-butyloxycarbonyl (Boc) group, or a PEG ether having from 1 to 5 ethylene glycol groups and terminating in a methyl group or ethyl group.

73. The copolymer of claim 71, wherein R₂ is an N-terminal alkyl amino group having from 1 to 5 carbon atoms, wherein the amino group is substituted with a methyl group, two methyl groups, or a tert-butyloxycarbonyl (Boc) group, or a PEG ether having from 1 to 5 ethylene glycol groups and terminating in a methyl group or ethyl group.

74. The copolymer of claim 71, wherein R₁ is an N-terminal alkyl amino group having 2 carbon atoms.

75. The copolymer of claim 71, wherein R₁ is an N-terminal alkyl amino group substituted with two methyl groups.

76. The copolymer of claim 71, wherein R₁ is an ether group having an alkyl group of 2 carbon atoms between the carboxylate oxygen and the ether oxygen and a methyl group attached to the ether oxygen.

77. The copolymer of claim 71, wherein R₁ is an alkyl group having four carbon atoms.

78. The copolymer of claim 71, wherein R₂ is an N-terminal alkyl amino group having 2 carbon atoms.

79. The copolymer of claim 71, wherein R₂ is an N-terminal alkyl amino group substituted with two methyl groups.

80. The copolymer of claim 71, wherein R₂ is an ether group having an alkyl group of 2 carbon atoms between the carboxylate oxygen and the ether oxygen and a methyl group attached to the ether oxygen.

81. The copolymer of claim 71, wherein R₂ is an alkyl group having four carbon atoms.

82. The copolymer of any of claims 71-81, further comprising:
c) from 10 to 90% by weight of a repeat unit derived from a monomer of formula (IX); 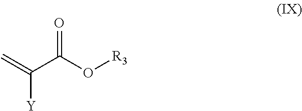
where Y is hydrogen or methyl; and
R₃ is an alkyl group having from 1 to 5 carbon atoms, an ether group having an alkyl group of from 1 to 5 carbon atoms between the carboxylate oxygen and the ether oxygen and a methyl or ethyl group attached to the ether oxygen, an N-terminal alkyl amino group having from 1 to 5 carbon atoms.

83. The copolymer of claim 82, wherein R₃ is an N-terminal alkyl amino group having from 1 to 5 carbon atoms, wherein the amino group is substituted with a methyl group, two methyl groups, or a tert-butyloxycarbonyl (Boc) group, or a PEG ether having from 1 to 5 ethylene glycol groups and terminating in a methyl group or ethyl group.

84. The copolymer of claim 82, wherein R₃ is an N-terminal alkyl amino group having 2 carbon atoms.

85. The copolymer of claim 82, wherein R₃ is an N-terminal alkyl amino group substituted with two methyl groups.

86. The copolymer of claim 82, wherein R₃ is an ether group having an alkyl group of 2 carbon atoms between the carboxylate oxygen and the ether oxygen and a methyl group attached to the ether oxygen.

87. The copolymer of claim 82, wherein R₃ is an alkyl group having four carbon atoms.

88. The copolymer of any of claims 82-87, further comprising:
d) from 10 to 90% by weight of a repeat unit derived from a monomer of formula (X); 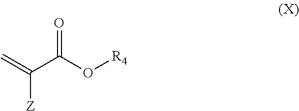
where Z is hydrogen or methyl; and
R₄ is an alkyl group having from 1 to 5 carbon atoms, an ether group having an alkyl group of from 1 to 5 carbon atoms between the carboxylate oxygen and the ether oxygen and a methyl or ethyl group attached to the ether oxygen, an N-terminal alkyl amino group having from 1 to 5 carbon atoms.

89. The copolymer of claim 88, wherein R₄ is an N-terminal alkyl amino group having from 1 to 5 carbon atoms, wherein the amino group is substituted with a methyl group, two methyl groups, or a tert-butyloxycarbonyl (Boc) group, or a PEG ether having from 1 to 5 ethylene glycol groups and terminating in a methyl group or ethyl group.

90. The copolymer of claim 82, wherein R₄ is an N-terminal alkyl amino group having 2 carbon atoms.

91. The copolymer of claim 82, wherein R₄ is an N-terminal alkyl amino group substituted with two methyl groups.

92. The copolymer of claim 82, wherein R₄ is an ether group having an alkyl group of 2 carbon atoms between the carboxylate oxygen and the ether oxygen and a methyl group attached to the ether oxygen.

93. The copolymer of claim 82, wherein R₄ is an alkyl group having four carbon atoms.

94. The copolymer of any of claims 71 to 93, wherein the copolymer further comprises at least two end groups.

95. The copolymer of claim 94, wherein each of the at least two end groups is independently selected from the group consisting of a monomer of formula (VII) to (X), a dithiobenzoyl group, and a 4-cyano-4-yl-pentanoic acid group.

96. The copolymer of any of claims 71 to 95, wherein the copolymer comprises a number average molecular weight (Mn) ranging from 3,000 to 55,000.

97. The copolymer of any of claims 71 to 96, wherein the copolymer has a dispersity (D) ranging from 1.10 to 1.50.

98. The copolymer of claim 97, wherein the copolymer has Ð ranging from 1.15 to 1.30.

99. The copolymer of any of claims 71 to 97, wherein the copolymer is a statistical copolymer, a random copolymer, a periodic copolymer, an alternating copolymer, a block copolymer, or a graft copolymer.

100. A polymer comprising:
a repeat unit derived from a monomer of formula (VII); 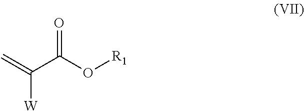
where W is hydrogen or methyl; and
R₁ is an alkyl group having from 1 to 5 carbon atoms, an ether group having an alkyl group of from 1 to 5 carbon atoms between the carboxylate oxygen and the ether oxygen and a methyl or ethyl group attached to the ether oxygen, an N-terminal alkyl amino group having from 1 to 5 carbon atoms.

101. The polymer of claim 102, wherein the amino group is substituted with a methyl group, two methyl groups, or a tert-butyloxycarbonyl (Boc) group, or a PEG ether having from 1 to 5 ethylene glycol groups and terminating in a methyl group or ethyl group.

102. The polymer of any of claims 71 to 101, wherein the polymer further comprises at least two end groups.

103. The polymer of claim 102, wherein each of the at least two end groups is independently selected from the group consisting of a monomer of formula (VII), a dithiobenzoyl group, and a 4-cyano-4-yl-pentanoic acid group.

104. The polymer of any of claims 71 to 103, wherein the polymer comprises a number average molecular weight (Mn) ranging from 3,000 to 55,000.

105. The polymer of any of claims 71 to 104, wherein the polymer has Ð ranging from 1.10 to 1.50.

106. The copolymer of claim 97, wherein the copolymer has Ð ranging from 1.15 to 1.30.

107. The polymer of any of claims 71 to 106, wherein the polymer is a linear polymer or a graft polymer.

108. A method for activating an NLRP3 inflammasome in a cell, the method comprising administering to the cell an effective amount of the polymer of any of claims 71-107.

109. The method of claim 108, wherein administering the polymer increases IL-1B production in the cell.

110. The method of any of claim 36-70 or 108-109, wherein the subject or cell is human.
